(19) United States
(12) Patent Application Publication
(10) Pub. No.: US 2021/0398647 A1
(43) Pub. Date: Dec. 23, 2021

(54) METHOD OF PROVIDING INFORMATION

(71) Applicant: Panasonic Intellectual Property Management Co., Ltd., Osaka (JP)

(72) Inventors: HIROSHI YAHATA, Osaka (JP); TAKAHIRO NISHI, Nara (JP); TADAMASA TOMA, Osaka (JP); TOSHIYASU SUGIO, Osaka (JP)

(21) Appl. No.: 17/467,517

(22) Filed: Sep. 7, 2021

Related U.S. Application Data
(63) Continuation of application No. PCT/JP2021/011001, filed on Mar. 18, 2021.

(30) Foreign Application Priority Data
Mar. 27, 2020 (JP) 2020-058035
Mar. 1, 2021 (JP) 2021-032149

(51) Int. Cl. G16H 20/60; G16H 10/60; G06Q 30/06; G06F 21/32; A61B 5/00

(52) U.S. Cl. CPC G16H 20/60; G16H 10/60; G06Q 30/0623; G16H 40/67; G06Q 30/0633; G06F 21/32; A61B 5/4866; G06Q 30/0631

(57) ABSTRACT

A method of providing information includes outputting a request of acquisition of menu information to a second server based on a request for a personalized menu for the user acquired from an information terminal of the user via a network, acquiring identification information stored in the information terminal from the information terminal via a network, acquiring the menu information from a second server generating information indicating a degree of dietary restriction depending on a progress of a disease of the user based on the biological information of the user, generating, based on the menu information and the information indicating the degree of dietary restriction, a personalized menu for the user so as to meet the information indicating the degree of dietary restriction, and transmitting the personalized menu to the information terminal, thereby allowing a dish to be selected from the personalized menu on the information terminal.

### BACKGROUND

### 1. Technical Field

The present disclosure related to a method of providing information.

### 2. Description of the Related Art

Japanese Unexamined Patent Application Publication No. 2005-222191 (hereinafter, for simplicity, referred to as PTL 1) discloses a technique for proposing a menu based on personal data including user's preference information, harmful ingredient information indicating an ingredient prohibited to be ingested, and health management information for intake management such that the menu complies with the personal data.

Japanese Unexamined Patent Application Publication No. 2008-299821 (hereinafter, for simplicity, referred to as PTL 2) discloses an order reception apparatus for use in a restaurant, which is installed in the restaurant for inputting menu order information and the like to support a customer service operation. The order reception apparatus for use in a restaurant disclosed in PTL 2 includes means for displaying an order input screen on a display device on which it is allowed to input menu order information. On the order input screen, menu order information is input individually for each seat set at a table.

### SUMMARY

A further improvement is required in the related technique described above.

In one general aspect, the techniques disclosed here feature a method of providing information in a health management system that includes a first server which manages biological information of a user in association with identification information identifying the user and that communicates with a second server that stores menu information indicating one or more dishes corresponding to a restaurant, the method including outputting a request of acquisition of the menu information to the second server based on a request for a personalized menu for the user acquired from an information terminal of the user via a network, acquiring the identification information stored in the information terminal from the information terminal via a network, acquiring the menu information from the second server, generating information indicating a degree of dietary restriction depending on a progress of a disease of the user based on the biological information of the user, generating, based on the menu information and the information indicating the degree of dietary restriction, a personalized menu for the user so as to meet the information indicating the degree of dietary restriction, and transmitting the personalized menu to the information terminal to allow a dish to be selected via the personalized menu on the information terminal.

A further improvement can be achieved by the above aspect.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a diagram showing a configuration of a common dish ordering system;

FIG. 2 is a diagram showing an example of an overall view of an information infrastructure of an information processing system according to an embodiment;

FIG. 3 is a diagram showing, in further detail, the overall view of the information processing system according to the present embodiment;

FIG. 4 is a diagram showing an example of a specific configuration of an information processing system according to an embodiment;

FIG. 5 is a diagram showing an example of a layout of a store of a restaurant company;

FIG. 6A is a diagram showing an example of a manner in which a QR code (registered trademark, the same hereinafter) is set on a seat;

FIG. 6B is a diagram showing an example of a manner in which a QR code is set on a seat;

FIG. 6C is a diagram showing an example of a manner in which a QR code is set on a seat;

FIG. 6D is a diagram showing an example of a manner in which a QR code is set on a seat;

FIG. 7 is a diagram showing an example of an operation screen which is displayed on an information terminal when a user operates the information terminal to read a QR code;

FIG. 8 is a diagram showing an example of an operation screen which is displayed on an information terminal immediately after a QR code reader reads a QR code;

FIG. 9 is a diagram illustrating an example of an operation screen including a standard menu provided by a restaurant company A;

FIG. 10 is a diagram showing an example of a scene in which a user operates an operation screen to order a dish via a standard menu;

FIG. 11 is a diagram showing an example of an operation screen which is displayed when a dish is selected from a standard menu and the selected dish is finally determined to be ordered;

FIG. 12 is a diagram showing an example of an authentication screen which is displayed on an information terminal immediately after a user, who wants to order a dish, starts a matching application;

FIG. 13 is a diagram showing another example of an authentication screen;

FIG. 14 is a diagram showing an example of a home screen which is displayed immediately after user authentication by a matching application is completed;

FIG. 15 is a diagram showing an example of an operation screen which is displayed on an information terminal when a user operates the information terminal to read a QR code at his/her seat after starting a matching application;

FIG. 16 is a diagram showing an example of an operation screen which is displayed on an information terminal when a restaurant ID and a seat ID are acquired via NFC;

FIG. 17 is a diagram showing an example of a display screen which is displayed on an information terminal when a matching application is generating a personalized menu;

FIG. 18 is a diagram showing an example of an operation screen including a personalized menu;

FIG. 19 is a diagram showing a scene in which a user operates an operation screen to select a dish from a personalized menu and order the selected dish;

FIG. 20 is a diagram showing an example of an operation screen which is displayed when a dish is selected from a personalized menu and the selected dish is finally determined to be ordered;

FIG. 21 is a diagram showing an example of an order history screen which is displayed in response to a request for seeing an order history issued by a user;

FIG. 22 is a table summarizing examples of dietary restrictions imposed on diabetic patients;

FIG. 23 is a graph for explaining an example of calculating a maximum acceptable intake of calories in a current meal;

FIG. 24 is a graph showing a relationship between a blood pressure and a salt intake of a hypertensive patient;

FIG. 25 is a diagram showing an example of a data structure of information including a diagnosis result of a user's periodic medical examination managed by a first server;

FIGS. 26A and 26B are each a diagram showing an example of a data structure of information including contents of a medical certificate of a user managed by a first server;

FIG. 27 is a diagram showing an example of a data structure of information including biological information managed by a first server;

FIGS. 28A, 28B, and 28C are each a diagram showing an example of a data structure of ingredient information;

FIGS. 29A, 29B, and 29C are each a diagram showing an example of ingredient information of low-salt ramen and vegetable dumplings;

FIG. 30 is a sequence diagram showing an example of an overall view of a process performed in an information processing system according to an embodiment;

FIG. 31 is a flowchart showing an example of a process which is performed by an information terminal when a dish is selected from a standard menu and the selected dish is ordered;

FIG. 32 is a flowchart showing an example of a process which is performed by an information terminal when a dish is selected from a personalized menu and the selected dish is ordered;

FIG. 33 is a flowchart showing details of a process in step S1 in FIG. 32;

FIG. 34 is a flowchart showing details of a process in step S2 in FIG. 32;

FIG. 35 is a flowchart showing details of a process in step S3 in FIG. 32;

FIG. 36 is a flowchart showing details of a process in step S4 in FIG. 32;

FIG. 37 is a diagram showing equation (1) for calculating information indicating the degree of dietary restriction;

FIG. 38 is a diagram showing equation (2) for calculating the priority of each dish;

FIG. 39 is a diagram showing an example of a specific implementation of an information processing system according to an embodiment;

FIG. 40 is a flowchart showing an example of a process performed on a file by a matching application from starting of the matching application to displaying of a personalized menu;

FIG. 41 is a diagram showing an example of an operation screen including a first variation of a personalized menu;

FIGS. 42A and 42B are each a diagram showing an example of an operation screen including a second variation of a personalized menu;

FIG. 43 is a diagram showing an example of an operation screen including a tile object displayed in a grayed-out manner;

FIGS. 44A and 44B are each a diagram showing an example of an operation screen including a third variation of a personalized menu;

FIG. 45 is a sequence diagram showing an example of a process of uploading biological information to a first server in a mode in which a personalized menu is generated by a second server;

FIG. 46 is a sequence diagram showing an example of a process performed by a first server to acquire disease information in a mode in which a personalized menu is generated by a second server;

FIG. 47 is a sequence diagram showing an example of a process performed in an information processing system when a user permits a second server to access disease information;

FIG. 48 is a sequence diagram showing an example of a process performed in an information processing system when a user prohibits a second server to access disease information;

FIG. 49 is a sequence diagram showing an example of a process of generating a personalized menu in an information processing system in a mode in which personalized menus are generated by a second server;

FIG. 50 is a sequence diagram showing an example of a process performed in an information processing system when permission of generating a personalized menu is refused in a mode in which personalized menus are generated by a second server; and

FIG. 51 is a sequence diagram showing an example of a process performed in an information processing system including a health management system according to a third embodiment.

### DETAILED DESCRIPTION

### Underlying Knowledge Forming Basis of the Present Disclosure

In recent years, the number of patients suffering from lifestyle-related diseases such as diabetes, hypertension, and hyperlipidemia has been increasing. Dietary restrictions are imposed on such patients to reduce their caloric intake, alcohol intake, etc., and they are required to pay attention to their daily diet to comply with these restrictions. However, while it is relatively easy to comply with such a dietary restriction at home because the amount of ingredients can be arbitrarily adjusted in cooking, it is not easy to comply with such a dietary restriction in eating out because the patient has to order a dish from the menu determined by the restaurant. If a restaurant can propose a menu mainly including dishes suitable for a patient by taking such dietary restrictions into consideration, it is possible for the restaurant to provide higher customer satisfaction and differentiate from other restaurants.

However, it is not easy to prepare a menu appropriate for users because the contents of dietary restrictions are different depending on a disease and the state of the disease of the patient.

In the technique disclosed in PTL 1 described above, a user enters a user ID and a password on a menu terminal provided for each table in a store, a store server acquires, from a data center, the user's personal data and store data of the store, the store server determines, based on the personal data and the store data, a menu of dishes including no problematic ingredients (harmful ingredients and ingredients the user does not like) and a recommended menu (of dishes including many favorite ingredients of the user), and the determined menus are displayed on the menu terminal.

The user's personal data is sensitive information, and thus it is not appropriate to provide the information to a third party without the user's permission.

However, the technique disclosed in PTL 1 has a problem that the personal data of the user is transmitted from the data center to the store server and thus the personal data is provided to the store without the permission of the user.

To solve the problems described above, the present disclosure is to provide a technique for preventing a user who is subject to a dietary restriction from being provided with a dish that does not meet the dietary restriction.

The present disclosure is to provide a technique for preventing sensitive information stored in a first server from being leaked to the outside of the first server without permission of a user.

In an aspect, the present disclosure provides a method of providing information in a health management system that includes a first server which manages biological information of a user in association with identification information identifying the user and that communicates with a second server that stores menu information indicating one or more dishes corresponding to a restaurant, the method including outputting a request of acquisition of the menu information to the second server based on a request for a personalized menu for the user acquired from an information terminal of the user via a network, acquiring the identification information stored in the information terminal from the information terminal via a network, acquiring the menu information from the second server, generating information indicating a degree of dietary restriction depending on a progress of a disease of the user based on the biological information of the user, generating, based on the menu information and the information indicating the degree of dietary restriction, a personalized menu for the user so as to meet the information indicating the degree of dietary restriction, and transmitting the personalized menu to the information terminal to allow a dish to be selected via the personalized menu on the information terminal.

In this aspect, a request of acquisition of menu information is output to the second server based on a request for a personalized menu issued by a user, and the menu information is acquired from the second server. Furthermore, based on biological information of the user, information is generated which indicates a degree of dietary restriction depending on a progress of a disease of the user. Based on the generated information indicating the degree of dietary restriction and the acquired menu information, the personalized menu for the user is generated according to the degree of dietary restriction described in the information. This personalized menu is displayed on the display of the information terminal. A dish is selected from the displayed personalized menu. As described above, in this aspect, the personalized menu meets the information indicating the degree of dietary restriction, and the user selects a dish from this personalized menu. Therefore, it is possible to prevent a user who is subject to a dietary restriction from being provided with a dish that does not meet the dietary restriction.

Furthermore, in the present aspect, since the biological information is not transmitted to the second server, it is possible to prevent the biological information from being leaked to the restaurant.

In the above-described method of providing information, when the request for the personalized menu for the user is acquired, a seat ID indicating a seat of the user may be acquired from the information terminal of the user, wherein the seat ID may be acquired via an operation screen displayed on a display of the information terminal.

If a dish ordered by a first customer sitting at the same table as a second customer who is a patient is mistakenly served to the second customer, the second customer will be uncomfortable. In this situation, if the mistakenly served dish does not meet the dietary restriction of the second customer and if the second customer eats this dish, the health of the second customer may be harmed. Therefore, such a serving mistake must be avoided.

In the technique disclosed in PTL 1, as can be clearly seen from the fact that a unique table number is assigned to each table with a menu terminal, ordering of dishes is performed in units of tables, and nothing is taken into consideration as to ordering of dishes in units of seats. Therefore, in the technique disclosed in PTL 1, when a dish is ordered by a customer sitting in a seat at a table, there is a possibility that the ordered dish is mistakenly served to a different seat at the same table where another customer who is a patient is sitting, and the mistakenly served dish possibly does not comply with the dietary restriction imposed on the patient.

In the order input screen disclosed in PTL 2, it is allowed to input and register dish order information via an input device for each seat at each table. This order input screen includes a seat position image including seat objects representing a plurality of seats, and a plurality of dish images corresponding to respective dish items. For example, a restaurant clerk touches a seat object corresponding to a particular seat in the seat position image, and then touches a dish image corresponding to a particular desired dish item. As a result, the dish item for a customer seated at the touched seat is selected.

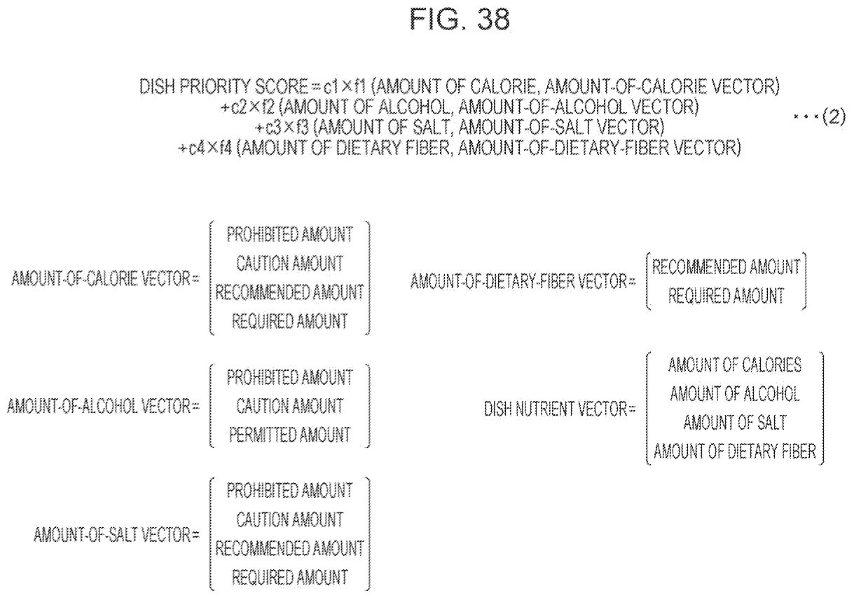

As described above, in the technique disclosed in PTL 2, the seat and the dish item are manually associated with each other by the clerk via the order input screen. Therefore, there is a possibility that a seat and a dish item are incorrectly associated by erroneous inputting. In particular, such erroneous inputting is likely to occur when the restaurant is crowded. Furthermore, the order input screen disclosed in PTL 2 includes various kinds of information and objects in addition to the seat position image and the dish images as shown in FIG. 38 of PTL 2. This may cause an increase in the possibility that an error occurs when inputting is performed on the order input screen disclosed in PTL 2. Therefore, as with the technique disclosed in PTL 1, the technique disclose in PTL 2 cannot prevent a possibility of an occurrence of mistaken serving of a dish.

In the aspect of the present disclosure, when a request for a personalized menu is acquired, a seat ID indicating a seat of a user is acquired via an operation screen. This makes it possible to automatically associate a dish ordered by a user via a personalized menu with a seat ID without a manual operation. Therefore, it is possible to prevent an occurrence of a mistaken serving of a dish, which may otherwise occur, for example in such a manner that when a dish is ordered by a user sitting at the same table as another user who has a dietary restriction, the ordered dish, which does not meet the dietary restriction, is mistakenly served to a seat of the user having the dietary restriction. Thus, it becomes possible to prevent a user having a dietary restriction from being brought into an uncomfortable situation. Furthermore, since such mistaken serving of dishes is prevented, it is possible to prevent a user who has a dietary restriction from eating a mistakenly served dish, which may cause the user to have a serious health problem.

In the above-described method of providing information, the personalized menu may include a dish produced by modifying a dish included in the menu information so as to reduce an ingredient specified by the information indicating the degree of dietary restriction as an ingredient to be avoided by the user.

In this aspect, the personalized menu may include a dish including a reduced amount of ingredient specified, in the information indicating the degree of dietary restriction, as an ingredient to be avoided by the user. This prevents a significant adverse effect on the user's health.

In the method of providing information, the reducing of the ingredient may include removing the ingredient such that an amount of the ingredient is zero.

In this aspect, the personalized menu may include a dish prepared such that an ingredient specified, in the information indicating the degree of dietary restriction, to be avoided by the user is completely removed. This more reliably prevents a significant adverse effect on the user's health.

In the method of providing information, the personalized menu may be generated from the menu information by excluding or graying out a dish including an ingredient specified by the information indicating the degree of dietary restriction as an ingredient to be avoided by the user.

In this aspect, the personalized menu is generated such that any dish including any ingredient specified, by the information indicating the degree of dietary restriction, to be avoided by the user is excluded or grayed out, and thus the user is allowed to smoothly order a dish while easily determining which dish includes some ingredients to be avoided or without caring whether or not the dish includes an ingredient to be avoided.

In the above-described method of providing information, the personalized menu may include a dish obtained by adding a nutrient, which tends to be deficient depending on the progress of the disease of the user, to a dish included in the one or more dishes.

In this aspect, the personalized menu includes a dish with an additional ingredient containing a nutrient that tends to be deficient depending on the progress of the disease of the user. Thus, it is possible to propose to the user a dish capable of supplementing a nutrient tending to be deficient, which is difficult for the user having a dietary restriction to identify due to a requirement of expert knowledge.

In the method of providing information, the personalized menu may include a description proposing a side dish for supplementing a nutrient tending to be deficient depending on the progress of the disease of the user.

In this aspect, the personalized menu includes a description proposing a side dish for supplementing a nutrient tending to be deficient depending on the progress of the disease of the user. Thus, it is possible to propose to the user a side dish capable of supplementing a nutrient tending to be deficient, which is difficult for the user having a dietary restriction to identify due to a requirement of expert knowledge.

In the method of providing information, the personalized menu may include a dish obtained by adding, to a dish included in the one or more dishes, an ingredient including a nutrient lacking in a past meal of the user in a particular period.

In the aspect, the personalized menu includes a dish obtained by adding, to a dish included in the one or more dishes, an ingredient including a nutrient lacking in a past meal of the user in a particular period. Thus, it is possible to propose to the user a dish capable of supplementing a nutrient which tends to be deficient, which is difficult for the user having a dietary restriction to identify due to a requirement of expert knowledge.

In the method of providing information, the personalized menu may include a description proposing a side dish for supplementing a nutrient lacking in a past meal of the user in a particular period.

In this aspect, the personalized menu includes a description proposing a side dish for supplementing a nutrient lacking in meals of the user in a past particular period. Thus, it is possible to propose to the user a side dish capable of supplementing a nutrient which tends to be deficient, which is difficult for the user having a dietary restriction to identify due to a requirement of expert knowledge.

In the method of providing information, the information indicating the degree of dietary restriction may include information indicating a maximum acceptable intake in a current meal as to a specific nutrient related to the disease of the user, and the information indicating the maximum acceptable intake in the current meal may be given by a value equal to or smaller than a value obtained by subtracting a cumulative intake taken in past one or more meals in a predetermined period from a maximum acceptable intake in the predetermined period, and the personalized menu may include a dish obtained such that when a dish included in the one or more dishes contains a greater amount of the specific nutrient than the maximum acceptable intake in the current meal, the amount of the specific nutrient is reduced to be equal to or smaller than the maximum acceptable intake in the current meal.

In this aspect, regarding the specific nutrient related to the disease, the information indicating the value obtained by subtracting the cumulative intake in past one or more meals in a predetermined period from the acceptable intake in the predetermined period is employed as the information indicating the maximum acceptable intake in the current meal. Furthermore, in a case where a dish includes a greater amount of specific nutrient than the maximum acceptable intake in the current meal, the amount of specific nutrient is reduced to be equal to or smaller than the maximum acceptable intake and the resultant dish is described on the personalized menu. As a result, a user with dietary restrictions can smoothly order a dish without caring about the amount of the specific nutrient that may exacerbate the disease.

In the method of providing information, the information indicating the degree of dietary restriction may include information indicating a target intake in a current meal as to a specific nutrient related to the disease of the user, and the information indicating the target intake in the current meal may be given by a value equal to or greater than a value obtained by subtracting a cumulative intake taken in past one or more meals in a predetermined period from an target intake in the predetermined period, and the personalized menu may include a dish obtained such that when a dish included in the one or more dishes contains a smaller amount of the specific nutrient than the target intake in the current meal, the amount of the specific nutrient is increased to be equal to or greater than the target intake in the current meal.

In this aspect, regarding the specific nutrient related to the disease, the information indicating the value obtained by subtracting the cumulative intake in past one or more meals in a predetermined period from the target intake in the predetermined period is employed as the information indicating the target intake in the current meal. When a dish includes a smaller amount of specific nutrient than the target intake in the current meal, the amount of specific nutrient is increased to be equal to or greater than the target intake and the resultant dish is described on the personalized menu. As a result, a user with a dietary restriction can smoothly order a dish without caring about the amount of the specific nutrient that may improve the disease.

In the method of providing information, the information indicating the degree of dietary restriction may include information indicating a maximum acceptable intake of calories in a current meal depending on the disease of the user, and the information indicating the maximum acceptable intake of calories in the current meal may be given by a value equal to or smaller than a value obtained by subtracting a cumulative intake of calories taken in past one or more meals in a predetermined period from an maximum acceptable intake of calories in the predetermined period, and the personalized menu may include a dish obtained such that when a dish included in the one or more dishes contains a greater amount of calories than the maximum acceptable intake of calories in the current meal, the amount of calories is reduced to be equal to or smaller than the maximum acceptable intake of calories in the current meal.

In this aspect, regarding the specific nutrient related to the disease, the information indicating the value obtained by subtracting the cumulative intake of calories in past one or more meals in a predetermined period from the acceptable intake of calories in the predetermined period is employed as the information indicating the maximum acceptable intake of calories in the current meal. Furthermore, in a case where a dish includes a greater amount of calories than the maximum acceptable intake of calories in the current meal, the amount of calories is reduced to be equal to or smaller than the maximum acceptable intake of calories and the resultant dish is described on the personalized menu. As a result, a user with a dietary restriction can smoothly order a dish without caring about the amount of calories to be taken in the meal.

In the method of providing information, the information indicating the degree of dietary restriction may include information indicating a maximum acceptable intake in a current meal as to a specific nutrient related to the disease of the user, and the information indicating the maximum acceptable intake in the current meal may be given by a value equal to or smaller than a value obtained by subtracting a cumulative intake taken in past one or more meals in a predetermined period from a maximum acceptable intake in the predetermined period, and the personalized menu may be generated such that when a dish in the one or more dishes includes a greater amount of the specific nutrient than the maximum acceptable intake in the current meal, the dish may be excluded or grayed out.

In this aspect, regarding the specific nutrient related to the disease, the information indicating the value obtained by subtracting the cumulative intake in past one or more meals in a predetermined period from the acceptable intake in the predetermined period is employed as the information indicating the maximum acceptable intake in the current meal. In a case where a dish includes a greater amount of the specific nutrient than the maximum acceptable intake of the specific nutrient in the current meal, the dish is excluded from the personalized menu or the dish is grayed out on the personalized menu. As a result, a user with a dietary restriction can smoothly order a dish after easily confirming that the dish does not include a nutrient that may exacerbate the disease or without caring whether or not the dish includes the nutrient that may exacerbate the disease.

In the method of providing information, the information indicating the degree of dietary restriction may include information indicating a target intake in a current meal as to a specific nutrient related to the disease of the user, and the information indicating the target intake in the current meal may be given by a value equal to or greater than a value obtained by subtracting a cumulative intake taken in past one or more meals in a predetermined period from an target intake in the predetermined period, and the personalized menu may be generated such that when a dish included in the or more dishes contains a smaller amount of the specific nutrient than the target intake in the current meal, the dish is excluded or grayed out.

In this aspect, regarding the specific nutrient related to the disease, the information indicating the value obtained by subtracting the cumulative intake in past one or more meals in a predetermined period from the target intake in the predetermined period is employed as the information indicating the target intake in the current meal. Furthermore, in a case where a dish includes a smaller amount of specific nutrient than the target intake in the current meal, the dish is excluded from the personalized menu or the dish is grayed out on the personalized menu. As a result, a user with a dietary restriction can smoothly order a dish after easily confirming that the dish includes a sufficient amount of specific nutrient that improves the disease or without caring whether or not the dish includes a sufficient amount of specific nutrient that improves the disease.

In the method of providing information, the information indicating the degree of dietary restriction may include information indicating a maximum acceptable intake of calories in a current meal depending on the disease of the user, and the information indicating the maximum acceptable intake of calories in the current meal may be given by a value equal to or smaller than a value obtained by subtracting a cumulative intake of calories taken in past one or more meals in a predetermined period from an maximum acceptable intake of calories in the predetermined period, and the personalized menu may be generated such that when a dish included in the one or more dishes contains a greater amount of calories than the maximum acceptable intake of calories in the current meal, the dish is excluded or grayed out.

In this aspect, regarding the specific nutrient related to the disease, the information indicating the value obtained by subtracting the cumulative intake of calories in past one or more meals in a predetermined period from the acceptable intake of calories in the predetermined period is employed as the information indicating the maximum acceptable intake of calories in the current meal. In a case where a dish includes a greater amount of calories than the maximum acceptable intake of calories in the current meal, the dish is excluded from the personalized menu or the dish is grayed out on the personalized menu. As a result, a user with a dietary restriction can smoothly order a dish after easily confirming that the dish does not include a greater amount of calories than the maximum acceptable intake or without caring calories of the dish.

In the method of providing information, the information indicating the degree of dietary restriction may include information indicating a maximum acceptable intake in a current meal as to a specific nutrient related to the disease of the user, and the information indicating the maximum acceptable intake in the current meal may be given by a value equal to or smaller than a value obtained by subtracting a cumulative intake taken in past one or more meals in a predetermined period from a maximum acceptable intake in the predetermined period, and the personalized menu may include a dish obtained such that when a dish included in the one or more dishes contains a greater amount of the specific nutrient than the maximum acceptable intake in the current meal, the dish is added with an ingredient containing a neutralizing nutrient having an effect that intake of the neutralizing nutrient together with the specific nutrient neutralizes an adverse effect of the greater amount of specific nutrient than the maximum acceptable intake.

In this aspect, regarding the specific nutrient related to the disease, the information indicating the value obtained by subtracting the cumulative intake in past one or more meals in a predetermined period from the acceptable intake in the predetermined period is employed as the information indicating the maximum acceptable intake in the current meal. In a case where a dish includes a greater amount of specific nutrient than the maximum acceptable intake of specific nutrient in the current meal, the dish is added with an ingredient including a nutrient that neutralizes adverse effects of the specific nutrient. As a result, a user with a dietary restriction can smoothly order a dish without caring about the adverse effects of the specific nutrient related to the disease.

In the method of providing information, the information indicating the degree of dietary restriction may include information indicating a maximum acceptable intake in a current meal as to a specific nutrient related to the disease of the user, and the information indicating the maximum acceptable intake in the current meal may be given by a value equal to or smaller than a value obtained by subtracting a cumulative intake taken in past one or more meals in a predetermined period from a maximum acceptable intake in the predetermined period, and the personalized menu may include a description of a combination of a dish including a greater amount of the specific nutrient than the maximum acceptable intake in the current meal and a dish including a neutralizing nutrient having an effect that intake of the neutralizing nutrient together with the specific nutrient neutralizes an adverse effect of the greater amount of the specific nutrient than the maximum acceptable intake.

In this aspect, regarding the specific nutrient related to the disease, the information indicating the value obtained by subtracting the cumulative intake in past one or more meals in a predetermined period from the acceptable intake in the predetermined period is employed as the information indicating the maximum acceptable intake in the current meal. In a case where a dish includes a greater amount of specific nutrient than the maximum acceptable intake in a current meal, the dish is combined with another dish including a nutrient having an effect of neutralizing the adverse effects of the specific nutrient, and the resultant combination is described on the personalized menu. As a result, a user with a dietary restriction can smoothly order a dish without caring about the adverse effects of the specific nutrient related to the disease.

In the method of providing information, the information indicating the degree of dietary restriction may include information indicating a maximum acceptable intake in a current meal as to a specific nutrient related to the disease of the user, and the information indicating the maximum acceptable intake in the current meal may be given by a value equal to or smaller than a value obtained by subtracting a cumulative intake taken in past one or more meals in a predetermined period from a maximum acceptable intake in the predetermined period, and the personalized menu may include a description of a combination of dishes that provides a total amount of intake equal to or smaller than the maximum acceptable intake in the current meal.

In this aspect, regarding the specific nutrient related to the disease, the information indicating the value obtained by subtracting the cumulative intake in past one or more meals in a predetermined period from the acceptable intake in the predetermined period is employed as the information indicating the maximum acceptable intake in the current meal. A combination of dishes including a total amount of specific nutrient equal to or smaller than the maximum acceptable intake in the current meal is displayed on the personalized menu. As a result, a user with a dietary restriction can smoothly order a combination of dishes without caring about the amount of the specific nutrient that may exacerbate the disease.

In the method of providing information, the information indicating the degree of dietary restriction may include information indicating a target intake in a current meal as to a specific nutrient related to the disease of the user, and the information indicating the target intake in the current meal may be given by a value equal to or greater than a value obtained by subtracting a cumulative intake taken in past one or more meals in a predetermined period from an target intake in the predetermined period, and the personalized menu may include a description of a combination of dishes that provides intake equal to or greater than the target intake in the current meal.

In this aspect, regarding the specific nutrient related to the disease, the information indicating the value obtained by subtracting the cumulative intake in past one or more meals in a predetermined period from the target intake in the predetermined period is employed as the information indicating the target intake in the current meal. A combination of dishes including a total amount of specific nutrient equal to or greater than the target intake in the current meal is displayed on the personalized menu. As a result, a user with a dietary restriction can smoothly order a combination of dishes without caring about the amount of specific nutrient that may improve the disease.

In the method of providing information, the information indicating the degree of dietary restriction may include information indicating a maximum acceptable intake of calories in a current meal depending on the disease of the user, and the information indicating the maximum acceptable intake of calories in the current meal may be given by a value equal to or smaller than a value obtained by subtracting a cumulative intake of calories taken in past one or more meals in a predetermined period from an maximum acceptable intake of calories in the predetermined period, and the personalized menu may include a description of a combination of dishes that provides a total amount of calories equal to or smaller than the maximum acceptable intake of calories in the current meal.

In this aspect, regarding the specific nutrient related to the disease, the information indicating the value obtained by subtracting the cumulative intake of calories in past one or more meals in a predetermined period from the acceptable intake of calories in the predetermined period is employed as the information indicating the maximum acceptable intake of calories in the current meal. Regarding to the specific nutrient, a combination of dishes including a total amount of calories equal to or smaller than the maximum acceptable intake of calories in the current meal is displayed on the personalized menu. As a result, a user with a dietary restriction can smoothly order a combination of dishes without caring about the amount of calories to be taken.

In the method of providing information, the personalized menu may include a first dish and a second dish that is displayed at a lower priority than the first dish, wherein the second dish may include a greater amount of nutrient whose intake is restricted due to the disease of the user than the first dish.

In this aspect, dishes are displayed on the personalized menu such that the more the dish includes a nutrient whose intake is restricted, the lower the priority of the dish in displaying it on the personalized menu. Thus, the user can easily recognize which dish has a high effect of improving the disease.

In the method of providing information, the personalized menu may include a first dish and a second dish that is displayed at a lower priority than the first dish, wherein the second dish may include a smaller amount of nutrient whose intake is recommended for the disease of the user than the first dish.

In this aspect, dishes are displayed on the personalized menu such that the more the dish includes an ingredient whose intake is recommended, the higher the priority of the dish in displaying it on the personalized menu. Thus, the user can easily recognize which dish has a high effect of improving the disease.

In the method of providing information, displaying of the second dish at the lower priority than the first dish may be executed in a manner including at least a) a manner in which an order of displaying the second dish is lower than an order of displaying the first dish, b) a manner in which a size of an area in which the second dish is displayed is smaller than a size of an area in which the first dish is displayed, or c) a manner in which the second dish is displayed in a lower gradation level than a gradation level in which the first dish is displayed.

In this aspect, the lower the priority of a dish, the lower the displaying order, or the lower the priority of the dish, the smaller the size of the area in which the dish is displayed, or the lower the priority of the dish, the lower the gradation level of displaying the dish. Thus, the user can easily recognize which dish is more effective in improving the disease.

In the method of providing information, the information indicating the degree of dietary restriction may be updated as the disease of the user progresses.

In this aspect, since the information indicating the degree of dietary restriction is updated according to the progress of the disease of the user, it is possible to generate information correctly indicating the degree of dietary restriction depending on the progress of the disease. Therefore, the personalized menu displayed can include appropriate dishes according to the progress of the disease of the user.

In the method of providing information, the information indicating the degree of dietary restriction may be updated according to at least one of a) an acquisition of a result of a medical examination, b) an acquisition of medical record information generated when a diagnosis is made, or c) an acquisition of biological information of the user via a biosensor that detects the biological information of the user.

In this aspect, since the information indicating the degree of dietary restriction is updated according to at least one of a) the acquisition of the result of the medical examination, b) the acquisition of medical record information generated when the diagnosis is made, or c) the acquisition of biological information of the user, it is possible to generate, based on the latest information, information indicating the degree of dietary restriction appropriate for the user.

The method of providing information may further include transmitting, to the first server, a) information indicating a dish name of a dish selected via the personalized menu, b) information indicating a price of the dish selected in via the personalized menu, c) information indicating a date and time when the dish selected via the personalized menu is ordered, and d) information indicating a nutrient included in the dish selected via the personalized menu.

Regarding the dish selected by the user from the personalized menu, a) the information indicating the name of the dish, b) the information indicating the price, c) the information indicating the date/time of the order, and d) the information indicating the nutrient are transmitted to the first server, and thus the first server can acquire the meal history of the user.

In the method of providing information, the identification information may include a user ID.

In this aspect, the identification information includes the user ID, and thus it is ensured that the biological information related to the user can be acquired from the first server.

In the method of providing information, the first server may be different from the second server.

The first server stores sensitive information related to the user such as biological information related to the user. It is not desirable that such sensitive information is provided outside the first server without the permission of the user. In this aspect, the first server is realized by a server different from the second server. Therefore, it is possible to prevent sensitive information on the user from being leaked to the outside of the first server.

In the method of providing information, the seat ID may be acquired by reading, via the operation screen, an identification code prepared at a corresponding position of a table where the user is seated.

In this aspect, the seat ID is acquired by reading the identification code prepared at the corresponding position of the table where the user is seated. Thus, it is possible to acquire the seat ID without having the user to manually input the information.

In the method of providing information, the identification code may include a QR code.

In this aspect, since the identification code is given by a QR code, it is possible to acquire the identification information without having the user to manually input the information.

In the method of providing information, the identification code may be read using NFC (Near Field Communication).

In this aspect, since the identification code is read using NFC, it is possible to acquire the identification information without having the user to manually input the information.

In the control method, the first server may manage in a distributed manner biological information, preference information of the user including goods purchase history information or dish order history information, and action history information including position information of the user.

In this aspect, the first server manages in a distributed manner the biological information, the preference information of the user including the goods purchase history information or the dish order history information, and the action history information including the position information of the user. This aspect can prevent these pieces of personal information from being leaked to the outside.

The method of providing information may be executed on an information terminal. The method of providing information may be realized by a program executed on a computer or a storage medium storing the program.

According to this aspect, it is possible to provide the information terminal for executing the control method, the program, and the storage medium storing the program.

In the program, the identification information that identifies the user may include a serial code of each information terminal assigned to the program.

According to this aspect, use of the serial code of each information terminal assigned to the program makes it possible to achieve the identification information with the information having higher confidentiality.

In another aspect, the present disclosure provides a method of providing information in a health management system communicating with a first server that manages biological information of a user in association with identification information identifying the user and including a second server and storing menu information indicating one or more dishes corresponding to a restaurant, the method including acquiring a seat ID indicating a seat of the user via a network from an information terminal of the user, wherein the seat ID is acquired via an operation screen displayed on a display of the information terminal, acquiring the identification information stored in the information terminal from the information terminal via a network, acquiring the biological information of the user via a network, generating information indicating a degree of dietary restriction depending on a progress of a disease of the user based on the biological information of the user, generating, based on the menu information and the information indicating the degree of dietary restriction, a personalized menu for the user so as to meet the information indicating the degree of dietary restriction, and transmitting the personalized menu to the information terminal, and allowing a dish to be selected from the personalized menu on the information terminal.

According to this aspect, when a dish is ordered, the seat ID indicating the seat of the user is acquired via the operation screen displayed on the display of the information terminal of the user, and the identification information identifying the user stored in the information terminal of the user is acquired via the network. Furthermore, the biological information of the user is acquired from the first server via the network, and a personalized menu for the user is generated so as to meet the information indicating the degree of dietary restriction based on the acquired biological information and menu information stored in the second server. This personalized menu is transmitted to the information terminal, and a dish is selected from this personalized menu.

As described above, in this aspect, in the series of processes in which the user orders a dish, the health management system acquires the seat ID indicating the seat of the user in a restaurant, and a dish is selected via the personalized menu. This personalized menu meets the information indicating the degree of dietary restriction. Thus, the associating of the ordered-dish information with the seat of the user is automatically performed without human intervention. Therefore, it is possible to prevent an occurrence of a mistaken serving of a dish, which may otherwise occur, for example in such a manner that when a dish is ordered by a user sitting at the same table as another user who has a dietary restriction, the ordered dish which does not meet the dietary restriction is mistakenly served to the seat of the user having the dietary restriction. Thus, it becomes possible to prevent a user having a dietary restriction from being brought into an uncomfortable situation. Furthermore, since such mistaken serving of dishes is prevented, it is possible to prevent a user who has a dietary restriction from eating a mistakenly served dish, which may cause the user to have a serious health problem.

### First Embodiment

It is expected that the Internet will continue to spread in our society and various sensors will be used in our lives. As a result, it is expected that in our future society, it will be capable of digitizing information on conditions and activities of individuals, as well as information on the entire city, including buildings and transportation networks, and it becomes possible to use such digitized information in computer systems. Digitized personal data (personal information) is accumulated in the cloud via a communication network, managed by an information bank as big data, and used for various purposes for individuals.

Such an advanced information society is called Society 5.0 in Japan. The advanced information society is a society in which economic development and solutions to social issues are expected to be achieved via an information infrastructure (a cyber-physical system) in which a real space (a physical space) and a virtual space (a cyber space) are highly integrated.

In such an advanced information society, when an individual makes a decision in various daily situations, big data including accumulated personal information is analyzed to get to know what the best choice for the individual in a specific situation is.

Embodiments are described below in which an economic efficiency and personal optimization (personalization) are achieved in an advanced information society in which such cyber-physical systems operate. More specifically, the following embodiments are described taking as an example eating of individuals in such an advanced information society.

An example of a personally optimized dish order system is a system in which menu information is transmitted from a terminal at a store of a restaurant to a personal information terminal, and a menu indicating dishes including no ingredient which is to be avoided by a user having a dietary restriction is displayed as a recommended menu on the mobile terminal. First, a general dish order system is described which is expected to be built in a society before the above-described advanced information society is realized.

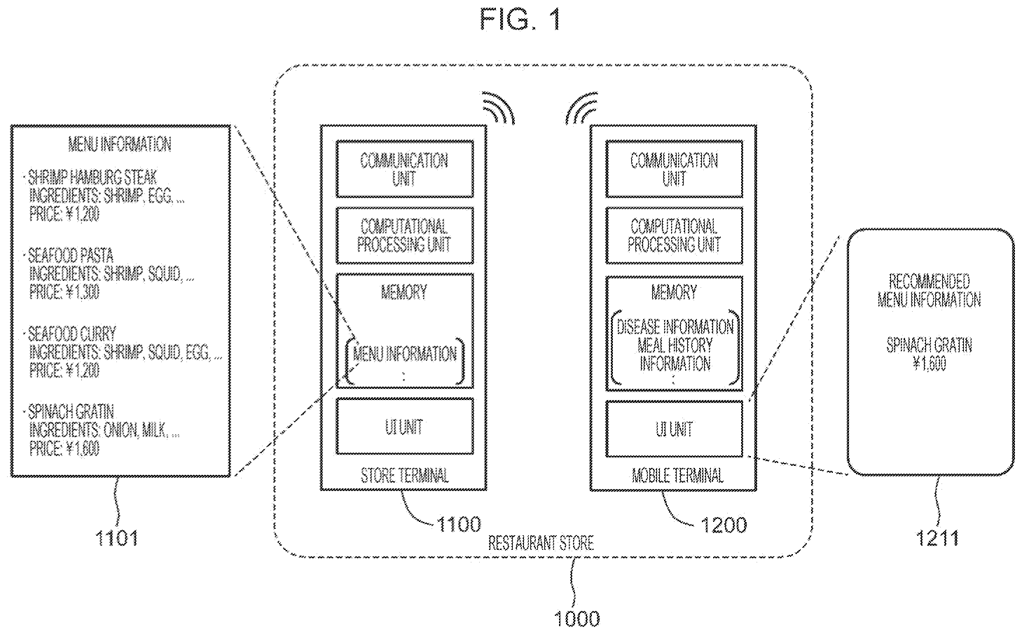

FIG. 1 is a diagram showing a configuration of a common dish ordering system. This dish ordering system includes a store terminal 1100 and a mobile terminal 1200. The store terminal 1100 and the mobile terminal 1200 are installed in a store of a restaurant 1000. The store terminal 1100 is a computer that transmits menu information. The store terminal 1100 includes a communication unit for communicating with an external apparatus, a computational processing unit for performing a computational process, a memory for storing data, and a UI unit for displaying and operating information. The memory stores menu information 1101. The menu information 1101 includes information on dishes served at the restaurant. More specifically, the menu information 1101 includes names of dishes, ingredients used in the dishes, and prices of the dishes. In the example shown in FIG. 1, the menu information 1101 includes four dishes: shrimp hamburg steak; seafood pasta; seafood curry; and spinach gratin.

The mobile terminal 1200 is a mobile terminal such as a smartphone owned by a user who visits the store 1000. The mobile terminal 1200 includes a communication unit for communicating with an external apparatus, a computational processing unit for performing a computational process, a memory for storing data, and a UI unit for displaying and operating information. The memory stores disease information, meal history information, and the like of a user who owns the mobile terminal 1200.

When the user enters the store 1000, the store terminal 1100 and the mobile terminal 1200 start communication automatically or manually. When the communication is started, the mobile terminal 1200 acquires menu information 1101 from the store terminal 1100. When the mobile terminal 1200 acquires the menu information 1101, the mobile terminal 1200 checks the menu information with respect to the disease information and meal history information stored in the memory, and extracts dishes that do not include ingredients which are to be avoided by the user. The mobile terminal 1200 generates a recommended menu 1211 based on the extracted dishes, and displays the generated recommended menu 1211 on the UI unit. In the example shown in FIG. 1, beef is an ingredient to be avoided by the user, and thus spinach gratin is selected as a beef-free dish and displayed in the recommended menu 1211.

According to the above-described technique, the user is allowed to select a dish containing no ingredient to be avoided from the displayed recommended menu.

In Society 5.0, personal information such as disease information, meal history information, and/or the like is centrally managed by a server operated by a company or an institution called an information bank that manages personal information in a concealed manner such that a third party is not capable of knowing individuals related to the personal information. This personal information is updated as required under the control of the information bank without needing a manual inputting operation by a user at a terminal.

However, in the order system shown in FIG. 1, disease information and meal history information are managed by the mobile terminal 1200, but it is not managed by the server. Therefore, in the order system shown in FIG. 1, it is not easy to update the disease information and the meal history information. For example, to update the disease information or the meal history information, the user needs to manually input the disease information or the meal history information to the mobile terminal 1200, which is troublesome for the user. Furthermore, since the disease information and meal history information are not concealed, there is a possibility that the disease information and meal history information are leaked to the store terminal 1100. Therefore, a further improvement is needed to adapt the order system shown in FIG. 1 to the advanced information society advocated by Society 5.0. Thus, the present embodiment provides an information processing system based on Society 5.0. The information processing system according to the embodiment of the present disclosure is described below with reference to drawings.

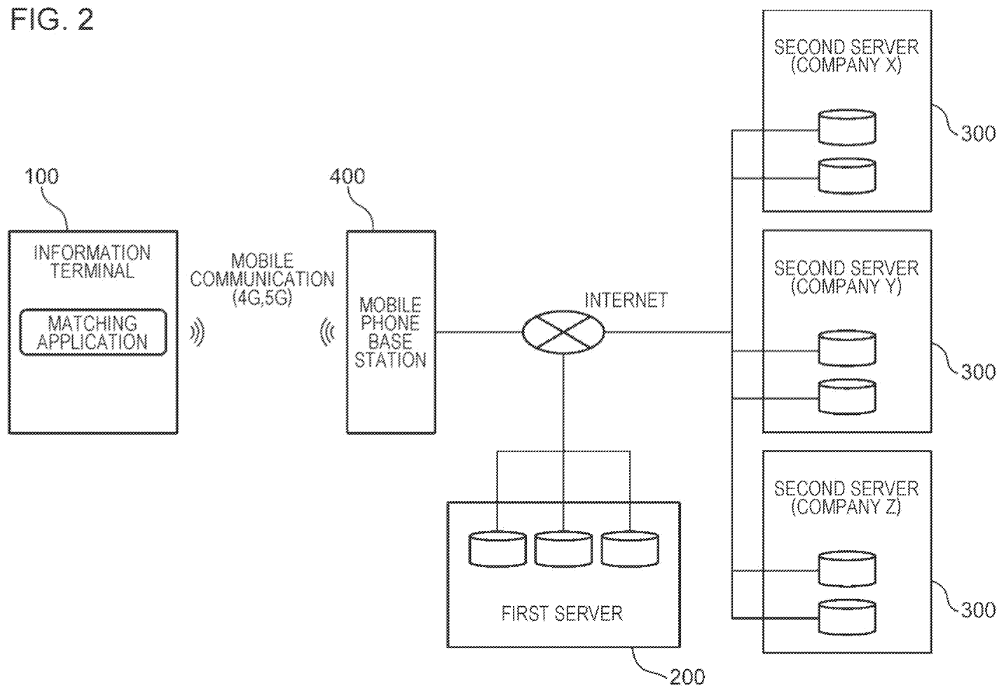

FIG. 2 is a diagram showing an example of an overall view of an information infrastructure of the information processing system according to the present embodiment. The information processing system shown in FIG. 2 is a system usable in Society 5.0, in which products or services suitable for a general consumer user are proposed based on personal information thereby providing a service of supporting the user to select a product or service. Before describing this selection support service that supports ordering of a dish according to the embodiment, an overview of an information infrastructure for realizing this embodiment is described with reference to FIG. 2. This information processing system mainly includes three apparatus groups.

A first apparatus group includes an information terminal 100 such as a smartphone owned by a user. A matching application is installed in the information terminal 100. The matching application is an application for selecting or recommending a product or service suitable for a user based on the user's personal information. The personal information used in the present disclosure broadly refers to public or non-public information about an individual. For example, the personal information includes at least one of the following: a name, a date of birth, an address, an annual income, an owned movable property/real estate information, physical information as to height/weight or the like, genetic information, medical information as to a medical history/medical record or the like, activity amount information as to the number of steps/calories burned, meal history information, vital sign information as to heartbeat/blood pressure or the like, purchase information via stores/EC sites or the like, word information as to words used in searching via Web search engine/AI speaker, information as text/video/audio transmitted or received via mail/SNS, or movement history information, or the like. The information terminal 100 is capable of connecting to the Internet, for example, via a mobile communication network called 4G or 5G and via a mobile phone base station 400.

The second apparatus group includes a first server 200. The first server 200 is a personal information server that stores personal information related to a user such that the personal information is decrypted and distributed at a plurality of locations. For example, the first server 200 manages personal information such that the personal information related to the user is fragmented, encrypted, and stored in a plurality of storage apparatuses located on the cloud. This ensures that high security is achieved, and leakage of the personal information is prevented. Furthermore, the first server 200 has a function of returning necessary data in response to an inquiry from a third party with the permission of the user. Furthermore, the first server 200 has a function of securely sharing the personal information permitted by a user with a company permitted by the user. That is, the first server 200 has a function as an information bank. In this case, for example, the first server 200 stores each one piece of data in a plurality of storage apparatuses in a distributed manner. An example of one piece of data is one file in which personal information is described.

In the present embodiment, the first server 200 allows a specific company to share specific personal information based on the permission of the user. Furthermore, the first server 200 has a function for providing a selection support service described below.

The above-described matching application is developed and/or provided by, for example, an operating company of the first server 200. This operating company evaluates the degree of suitability of the products or services that the user may use based on the personal information of the user. The operating company of the first server 200, the developing company of the matching application, and the company that distributes the matching application may be the same or different. The information processing system shown in FIG. 2 provides a selection support service using the matching application described above. Note that this merely an example, and the information processing system may be configured in various different manners. For example, the selection support service may be realized by using an application other than the matching application or a general browser or the like. To securely handle the personal information related to the user, it is preferable to provide the selection support service by a dedicated application such as the matching application. However, when personal information with low security requirement such as publicly available personal information is handled, or when a function for ensuring security is provided, means other than the matching application may be used in the selection support services.

The matching application handles personal information only inside the information terminal 100. For example, the matching application presents a user with a product or service that seems to be most suitable for the user under a given arbitrary condition in terms of time, a place, a situation, and/or the like. For example, the matching application provides an intermediary/mediation function in economic activities such as purchasing by a user.

The matching application is an application that opens the recommendation function to the public, which has been siloed for each service provider. This is described further below taking as an example a certain service provider which is famous in an electronic commerce market such as an electronic commerce (EC) site. Many products are listed on a site of the service provider. When a particular product is searched for or purchased, other products highly relevant to that product (for example, products that are often purchased together) are recommended to the user. The recommendation function for such purchases is effective only in the EC site of the service provider. Therefore, the recommendation function has no effect when products are purchased, meals are ordered at a restaurant, or planning a family vacation trip etc., via an EC site operated by another service provider.

It is expected that, in the future, personal information will be aggregated in information banks, and a mechanism will be established which makes it possible for anyone to access a huge variety of accurate personal information accumulated over a long period of time under predetermined conditions. In such a situation, the degree of suitability can be estimated not only for products provided by a service provider but also for all products or services based on a search or purchase history on the EC site of one service provider and based on the personal information of various users. This makes it possible to recommend products or services that are more valuable to the user from among various options.

According to the present embodiment, the first server 200 is assumed to be a general-purpose storage apparatus provided on a cloud that manages personal information in a distributed and encrypted manner so as to realize the above idea or function.

The third apparatus group is a group including a second server 300 by which each company manages data specific to the company. In the example shown in FIG. 2, three companies, company X, company Y, and company Z each own a second server 300, and manage and/or provide information about their products and/or their services. Note that companies are not limited to restaurant companies which are described by way of example in detail in the present disclosure. For example, the company may be a ready-made meal company such as a lunch box shop or a fast-food shop that provides takeaway food which has been cooked. The company may be a company which focuses on food to be cooked at home, such as a supermarket. Furthermore, the company may be even an automobile manufacturer, a real estate company, a hospital, a school, a cram school for studying or sports, a law firm, or a company that provides products and/or services to general consumers.

One of effects of the information processing system according to the present embodiment is that personal information is not given to the company without permission of a user. It is assumed that information banks allow specific companies to share personal information based on permission of users.

However, it is very troublesome to let users make judgment individually. Even if there is a trust company that sets a data operation policy, users may not be able to grasp exactly which data was passed to whom, which may cause the users to feel uneasy.

In view of the above, in this embodiment, it is prohibited for the company operating the first server 200 to use the stored personal information, for example, by decrypting and interpreting the personal information, unless the user permits.

Furthermore, in a case where there is an information bank or information intermediary that is operating in a market such that it manages personal information and provides a matching application under a strict privacy protection policy, a user may make a contract for receiving the service from this information bank or information intermediary. This makes it possible to prevent personal information from being given to other companies.

The present embodiment provides an information processing system for use in a next-generation information society in which a huge amount of personal information that changes from moment to moment can be used for matching with various services. The information processing system described below is assumed to be used in such a situation.

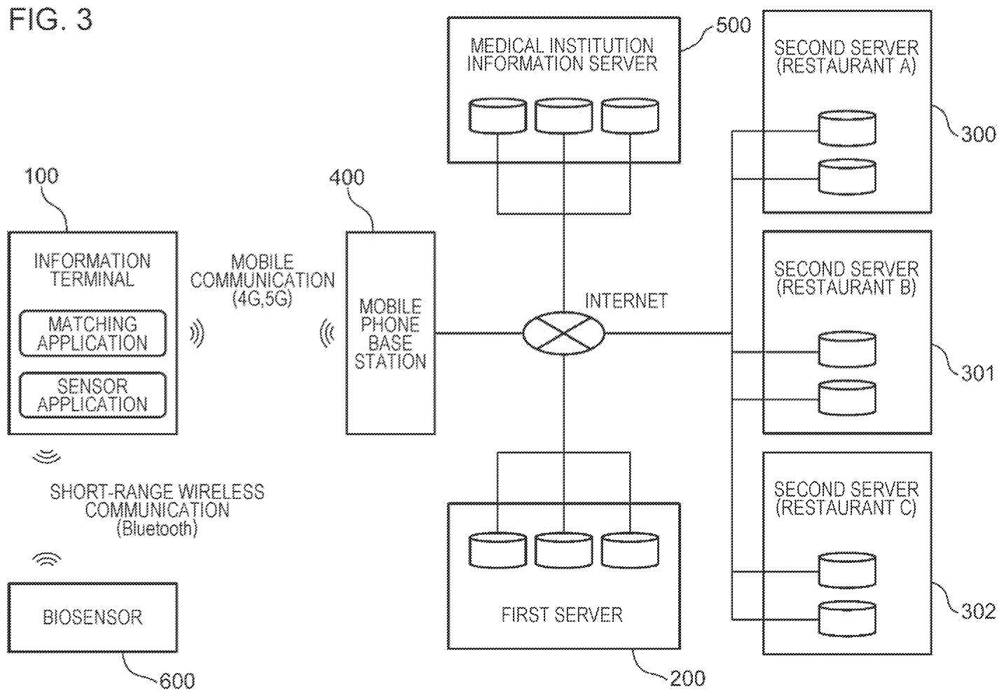

FIG. 3 is a diagram illustrating further details of the overall view of the information processing system according to the present embodiment. The information processing system shown in FIG. 3 is configured to provide menu information, in a restaurant or the like, for being seen by a user to order a dish such that the menu information is matched with the personal information related to the user and a menu optimized for the user is presented. The information processing system shown in FIG. 3 further includes a biosensor 600 and a medical institution information server 500 in addition to constituent elements of the information processing system shown in FIG. 2.

In this example, the service providing companies include a restaurant company A, a restaurant company B, and a restaurant company C, which are companies in the restaurant industry. Restaurant companies A, B, and C are separate independent companies. The information processing system shown in FIG. 3 includes a second server 300 operated by the restaurant company A, a second server 300 operated by the restaurant company B, and a second server 300 operated by the restaurant company C. Menu information provided by each restaurant and information about each store are managed by corresponding one of these second servers 300. The second servers 300 are each realized, for example, by a cloud server.

The medical institution information server 500 is a server managed by a medical institution such as a hospital or a clinic. The medical institution information server 500 manages information indicating a result of diagnosis of the user's periodic medical examination, information indicating a medical certificate issued by a hospital, and/or the like. The medical institution information server 500 is connected to the Internet. The information managed by the medical institution information server 500 is used in matching, as necessary.

The biosensor 600 is a sensor for acquiring biological information. The biological information acquired includes information about, for example, a heart rate, a blood pressure, a blood oxygen concentration, a blood sugar (glucose) level, HbAlc, breathing, a body temperature, an amount of water, calorie intake, an acceleration, the number of steps, activity/calory consumption, smell, myoelectricity, brain waves, a sleeping state, bioimpedance, and a urine salt concentration. Biosensors capable of sensing these pieces of biological information have already been put into practical use.

For example, the calorie intake and calorie consumption can be estimated from values detected by a plurality of different sensors, for example, the acceleration acquired by the acceleration sensor, the heart rate acquired by the heart rate sensor, and the blood sugar level acquired by the blood sugar level sensor.

For a user whose total calorie intake is restricted, for example, as in a case where the user suffers from gout, diabetes, or the like, the calorie intake of the user can be estimated in real time from a value output from the biosensor 600. This makes it possible to restrict a meal (stop eating more) and recommend low-calorie dishes to the user.

The blood sugar level or HbA1c can be measured using a sensor that measures, in real time, the sugar concentration in a subcutaneous interstitial fluid. Alternatively, the blood sugar level or HbA1c can be measured using a sensor that collects a small amount of blood from a user's fingertip or the like. Alternatively, HbA1c can be measured by an HbA1c measuring device installed in a pharmacy or a station.

The urine salinity can be obtained by a urine salinity sensor. The urine salinity sensor can measure the salt intake on the previous day by measuring the salt concentration of urine in the early morning. The urine salinity is measured for a user who is restricted in terms of salt intake as in a case where the user has hypertension or diabetes.

By detecting a small amount of alcohol emitted from the skin using an odor sensor, it is possible to detect whether or not a user has drunk. Alcohol detection is performed for a user who is subject to a dietary restriction on alcohol, as in a case, for example, where the user is a gout patient.

Information indicating the blood pressure, the calorie intake, the salt intake, the blood sugar level, and/or the like estimated from the data acquired from the biosensor 600 or a combination of the acquired data is sequentially uploaded as biological information to the first server 200 and accumulated therein. The uploaded biological information is used for improving the lifestyle of the user in terms of eating, exercising, and/or the like.

The biosensor 600 may be realized on a smart watch or the like. The biosensor 600 may be worn by a user who owns the information terminal 100. The biosensor 600 continuously measures the user's vital biological information. The biological information measured by the biosensor 600 is transmitted from the biosensor 600 to the information terminal 100 via short-range wireless communication such as Bluetooth (registered trademark) communication. The biological information is stored and/or managed by a sensor application installed on the information terminal 100. The sensor application uploads the acquired biological information and time information indicating the measurement time of the biological information to the first server 200 according to the user account information. Thus, the biological information is accumulated in the first server 200.

The sensor application may grant an access right to data stored and/or managed by the sensor application to the matching application or an OS (Operating System) of the information terminal 100. In this case, the biological information is uploaded to the first server 200 via the matching application or the OS. The sensor application may store the biological information in a memory of the information terminal 100.

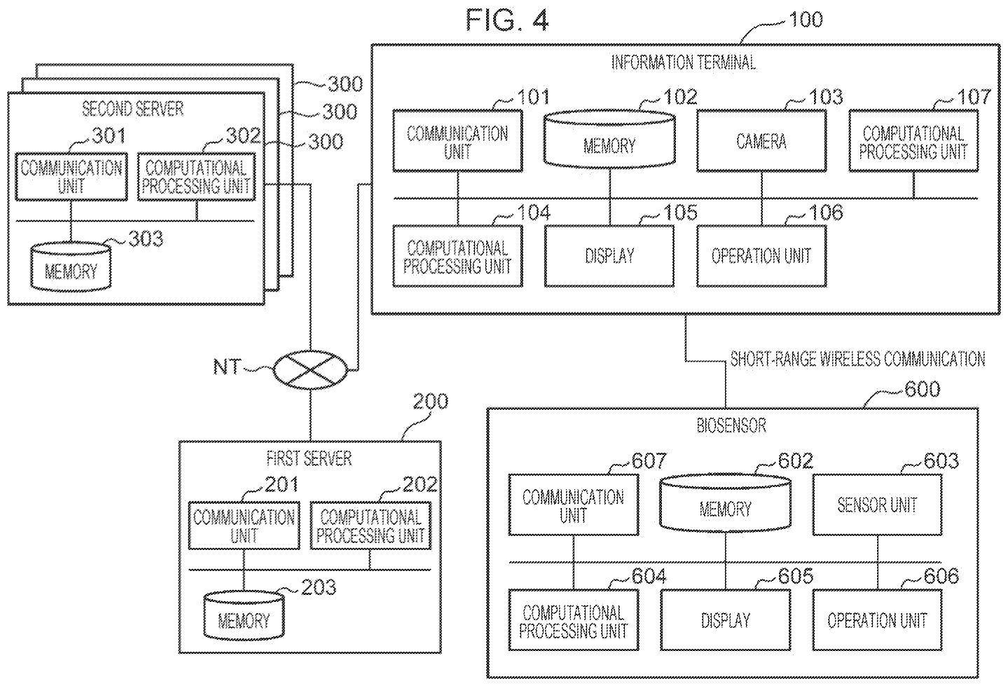

FIG. 4 is a diagram showing a specific example of a configuration of the information processing system according to the present embodiment. The information processing system shown in FIG. 4 includes the information terminal 100, the first server 200, the second server 300, and the biosensor 600 described above with reference to FIGS. 2 and 3. In FIG. 4, for convenience of explanation, the mobile phone base station 400 and the medical institution information server 500 are not shown. The information terminal 100, the first server 200, the second server 300, and the biosensor 600 are connected to each other so as to be capable of communicating with each other via a network NT. The network NT is a wide area communication network including a mobile phone communication network and the Internet.

The information terminal 100 is realized by a mobile information processing apparatus such as a smartphone, a tablet terminal, or the like. In the present embodiment, the information terminal 100 is carried by a user, which allows the user to order a dish at a store of a restaurant. The information terminal 100 includes a communication unit 101, a memory 102, a camera 103, a computational processing unit 104, a display 105, an operation unit 106, and a short-distance communication unit 107.

The communication unit 101 includes a communication circuit that connects the information terminal 100 to the network NT. The communication unit 101 receives menu information (described later) transmitted from the second server 300, and stores it in the memory 102. The computational processing unit 104 reads the menu information stored in the memory 102 and performs processing thereon. Furthermore, the communication unit 101 receives biological information and/or disease information (described later) from the first server 200 and stores it in the memory 102. Furthermore, under the control of the computational processing unit 104, the communication unit 101 transmits ordered-dish information (described later) and a seat ID (described later) in association with each other to the second server 300.

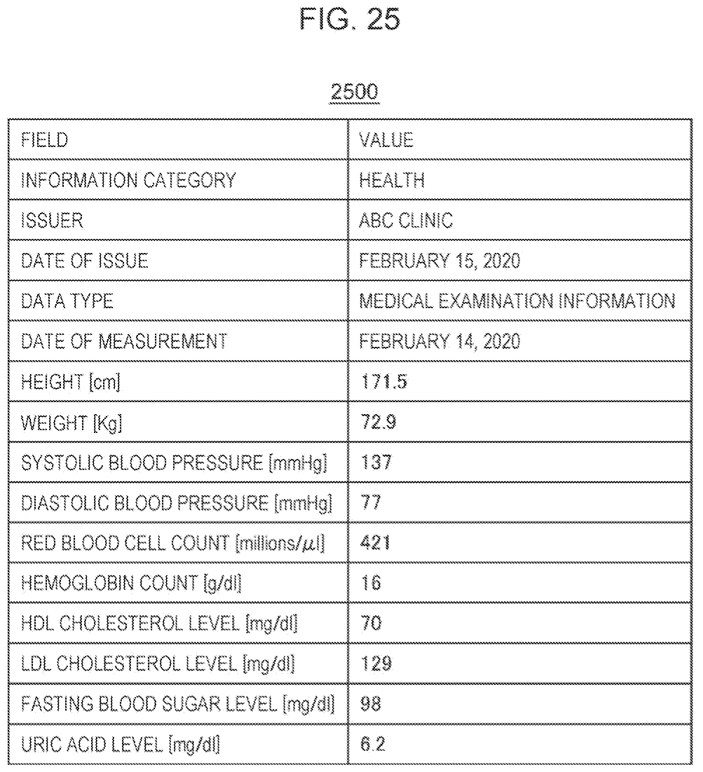

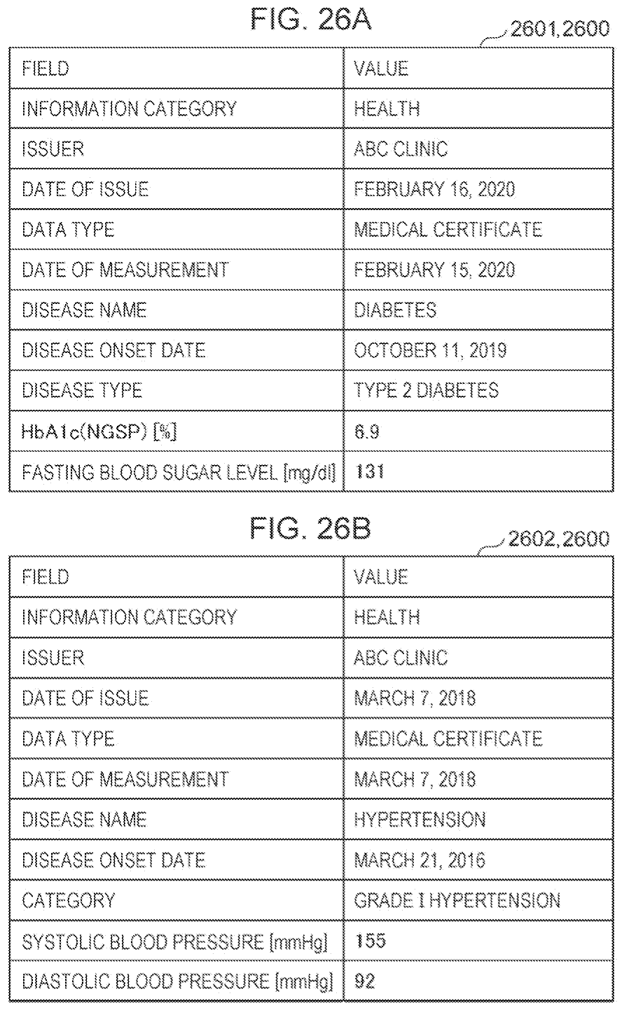

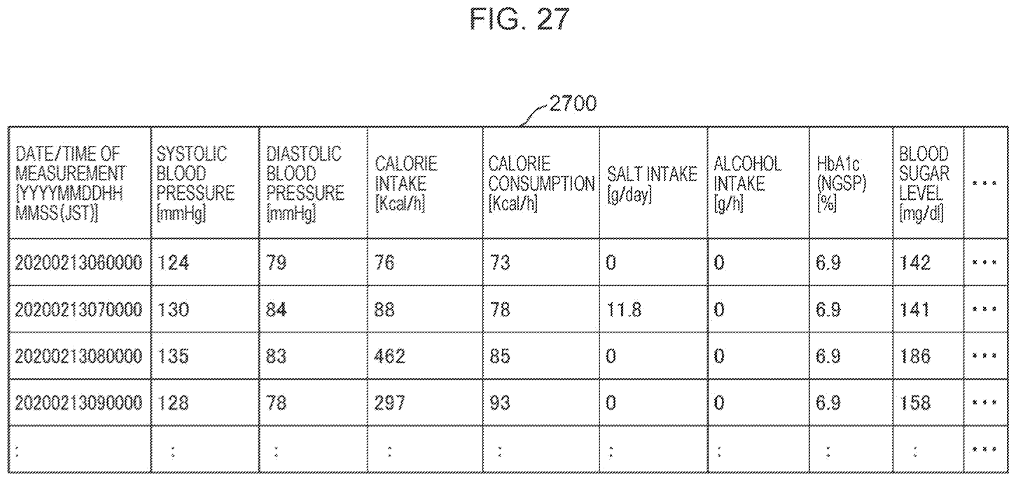

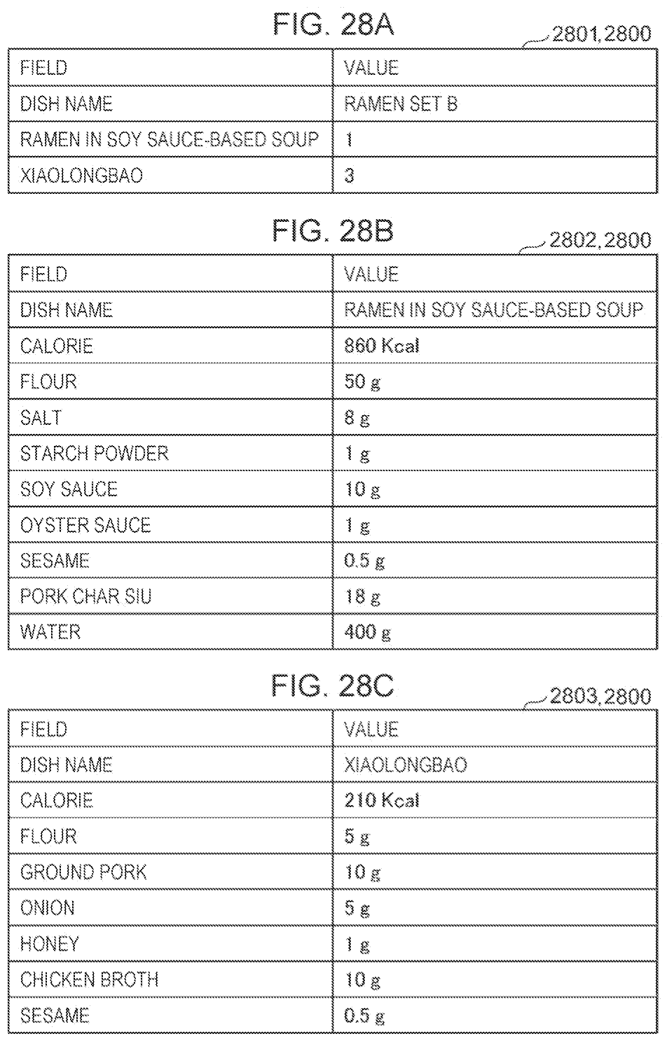

The memory 102 is realized using a nonvolatile storage apparatus such as a flash memory or the like. The memory 102 stores information 2500 including information on the diagnosis result of the periodic medical examination shown by way of example in FIG. 25 and information 2600 including information on the description contents of the medical certificate shown by way of example in FIGS. 26A and 26B and FIG. 27. The information 2500 and the information 2600 constitute the disease information transmitted from the first server 200. Furthermore, the memory 102 stores information 2700 including the biological information shown by way of example in FIG. 27 transmitted from the first server 200. Furthermore, the memory 102 stores ingredient information 2800 shown by way of example in FIGS. 28A to 28C transmitted from the second server 300.

The ingredient information 2800 is information about ingredients used in a dish, and one piece of ingredient information 2800 corresponds to one dish. The menu information includes one or more pieces of ingredient information 2800. Details of the information 2500, the information 2600, the information 2700, and the ingredient information 2800 will be described later. The memory 102 also stores identification information identifying a user. The identification information includes a user ID (Identifier). The user ID is an identifier of a user.

The camera 103 is an image capturing apparatus including a CMOS sensor and/or the like. The camera 103 is used to capture a QR code or the like attached to a seat in a store of a restaurant.

The computational processing unit 104 is realized using a processor such as a CPU. The computational processing unit 104 executes, on the information terminal 100, the OS, the above-described matching application, the QR code reader, the browser, and the like.

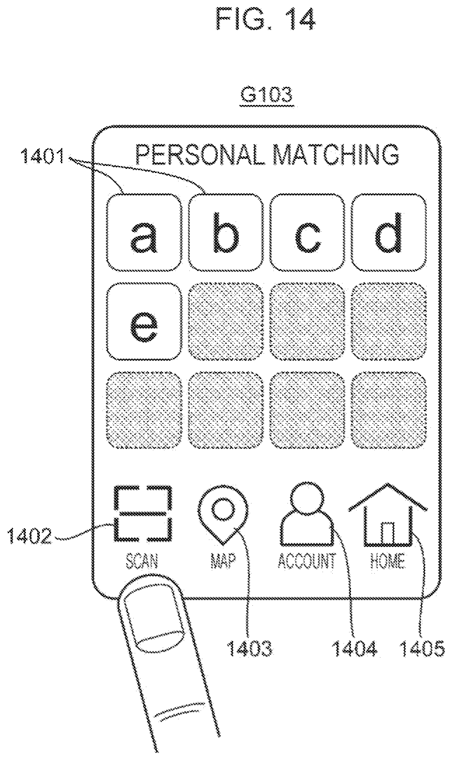

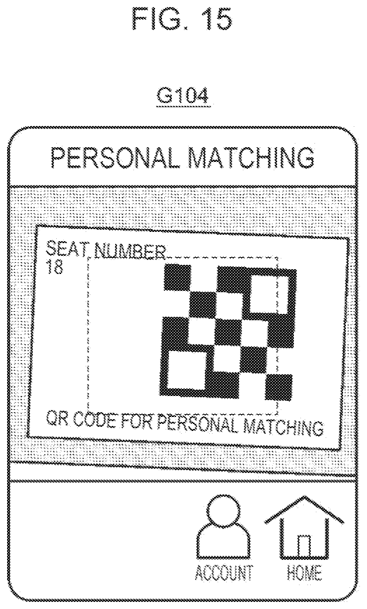

The computational processing unit 104 acquires a restaurant ID and a seat ID indicating a seat of a user via a first operation screen displayed on the display 105. The first operation screen is, for example, an operation screen G104 for reading a QR code provided by a matching application such as that shown in FIG. 15 or an operation screen G1011 for reading information by NFC as shown in FIG. 16. The restaurant ID is an identifier of a restaurant. When a restaurant has a plurality of stores, the restaurant ID may include a restaurant identifier and a store identifier. The seat ID is an identifier of a seat in a store. The computational processing unit 104 may acquire the restaurant ID and the seat ID by analyzing the QR code which is captured by the camera 103 in response to a shoot command issued by a user via the operation unit 106. Alternatively, in a case where the information terminal 100 is brought close to one of NFC IC chips disposed in respective seats of a restaurant when the matching application is in operation, the computational processing unit 104 may acquire the restaurant ID and the seat ID from the one IC chip via the short-distance communication unit 107.

The computational processing unit 104 acquires, via the network NT, menu information indicating one or more dishes available at the restaurant from the second server 300 of the restaurant corresponding to the restaurant ID, and stores the menu information in the memory 102. For example, in a case where the restaurant ID includes the identifier of the restaurant company A, the menu information is acquired from the second server 300 of the restaurant company A.

The computational processing unit 104 transmits identification information for identifying a user stored in the memory 102 to the first server 200. When biological information and/or disease information of the user is acquired based on the identification information from the first server 200, the computational processing unit 104 stores the acquired information and/or disease information in the memory 102.

The computational processing unit 104 generates information indicating the degree of dietary restriction depending on the progress of the disease of the user, based on the acquired menu information and the acquired biological information and/or disease information related to the user.

The computational processing unit 104 generates a personalized menu for the user so as to meet the generated information indicating the degree of dietary restriction. The personalized menu may include, for example, a dish obtained by modifying a dish included in the menu information acquired by the computational processing unit 104 so as to reduce the amount of an ingredient specified by the information indicating the dietary restriction as an ingredient to be avoided by the user. This allows the user to smoothly order a dish including no ingredients to be avoided.

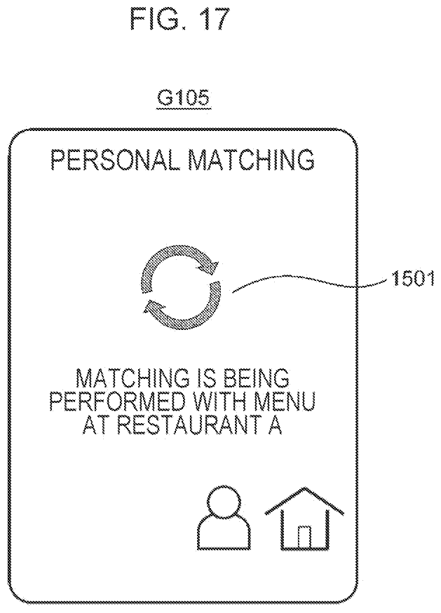

The computational processing unit 104 displays the personalized menu on a second operation screen, displayed on the display 105, for accepting an order of a dish in the restaurant. The second operation screen is an operation screen G106 such as that shown in FIG. 18 for a restaurant to accept a dish order from a user. The second operation screen is provided via the matching application based on a design specified by the restaurant. The user performs an input operation to select a desired dish from the personalized menu displayed on the second operation screen thereby ordering the dish.

The computational processing unit 104 transmits ordered-dish information indicating the dish selected from the personalized menu and the seat ID in association with each other to the second server 300 via the communication unit 101. The ordered-dish information and the seat ID transmitted to the second server 300 are displayed on the display installed in the store of the restaurant corresponding to the second server 300 which is the transmission destination. An employee of this store cooks the food ordered by the user according to the displayed contents and carry the cooked food to the user's seat. Thus, the user can get and eat the ordered dish.

The display 105 is realized, for example, by a liquid crystal display panel or an organic EL panel, and is used to display various images. For example, the display 105 displays the above-described first operation screen and second operation screen.

The operation unit 106 is realized using an input device such as a touch panel. The operation unit 106 receives an instruction issued by a user to select a desired dish from the personalized menu.

The short-distance communication unit 107 includes a communication circuit having an NFC communication function, and can write and read information into or from an IC chip having an NFC communication function. Furthermore, the short-distance communication unit 107 also includes a communication circuit having a Bluetooth (registered trademark) communication function, and communicates with the biosensor 600.

The configuration of the information terminal 100 has been described above.

Next, a configuration of the first server 200 is described below. The first server 200 includes a communication unit 201, a computational processing unit 202, and a memory 203. The communication unit 201 includes a communication circuit for connecting the first server 200 to the network NT. The communication unit 201 transmits the biological information and/or the disease information to the information terminal 100 in response to a request from the information terminal 100. The computational processing unit 202 is realized by a processor such as a CPU. The computational processing unit 202 processes personal information related to a user stored in the memory 203.

For example, let it be assumed here that the computational processing unit 202 receives, from the communication unit 201, a request signal for requesting acquisition of personal information about a permissive user. The permissive user refers to a user who has granted permission to read the personal information stored in the first server 200 in response to a request from the information terminal 100 or the second server 300 wherein the permission may be granted directly by the user or indirectly via a trusted third party. In this case, in response to the request from the information terminal 100 or the second server 300, the computational processing unit 202 reads out the personal information on the permissive user stored in the memory 203 and returns it via the communication unit 201. The personal information read in this situation may be the entire managed personal information, or only part of the entire information on a requested specific item (part of the personal information).

The memory 203 includes a plurality of nonvolatile storage apparatuses such as hard disk drives. The memory 203 stores personal information related to one or more users. The personal information includes the biological information and/or the disease information of each user. The personal information is stored such that it is encrypted and distributed over the plurality of storage apparatuses.

The personal information stored in the memory 203 may include biological information, preference information, and action history information. The biological information is information of each user acquired by the biosensor 600. The preference information includes purchase history information and/or order history information. The purchase history information is information indicating the purchase history of products (goods) or services of each user. The order history information is information indicating a history of dish orders of each user. The action history information is information indicating a history of actions of each user. The action history information includes, for example, time-series data in association with the user's position information and time information.

Next, a configuration of the second server 300 is described below. There is one or more second servers 300 corresponding to respective restaurant companies. The second server 300 includes a communication unit 301, a computational processing unit 302, and a memory 303. The communication unit 301 includes a communication circuit for connecting the second server 300 to the network NT. The communication unit 301 transmits menu information to the information terminal 100 in response to a request from the information terminal 100. The computational processing unit 302 is realized using a processor such as a CPU. The computational processing unit 302 processes the menu information stored in the memory 303. The memory 303 is realized using a nonvolatile storage apparatus such as a hard disk drive. The memory 303 stores menu information.

Next, a configuration of the biosensor 600 is described below. The biosensor 600 includes a communication unit 607, a memory 602, a sensor unit 603, a computational processing unit 604, a display 605, and an operation unit 606. The communication unit 607 includes a communication circuit for allowing the biosensor 600 to communicate with the short-distance communication unit 107 of the information terminal 100 via short-range wireless communication. As for the short-range wireless communication, for example, Bluetooth (registered trademark) can be used. The communication unit 607 transmits the biological information measured by the sensor unit 603 to the information terminal 100 via the short-range wireless communication.

The memory 602 is realized using a rewritable nonvolatile storage memory such as a flash memory or the like. The memory 602 stores, for example, biological information measured by the sensor unit 603. The sensor unit 603 includes a blood pressure sensor, an acceleration sensor, a heart rate sensor, a bioimpedance sensor, a blood sugar level sensor, a urine salt concentration sensor, a biogas sensor, an infrared sensor, a mid-infrared laser sensor, an odor sensor, and a piezoelectric sensor, and/or the like.

The computational processing unit 604 is realized using a processor such as a CPU, and is responsible for the overall control of the biosensor 600. The display 605 is realized, for example, by a liquid crystal panel or an organic EL panel, and is used to display biological information measured by the sensor unit 603. The operation unit 606 accepts various operations performed by a user.

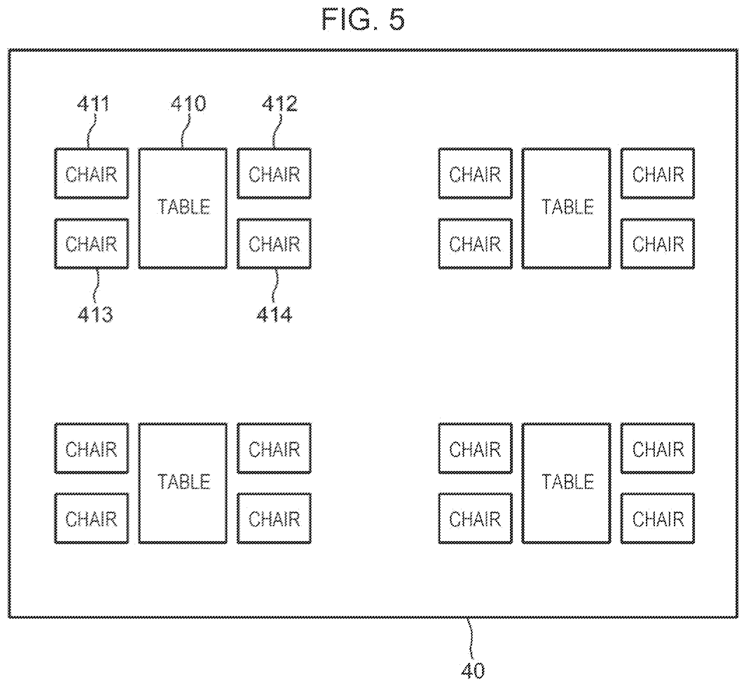

FIG. 5 is a diagram showing a layout of a store of a restaurant company. In the example in FIG. 5, a layout of a store 40 of the restaurant company A is shown. Four tables 410 are installed in the store 40. Four chairs 411, 412, 413, and 414 are installed at each table 410.

When two or more users are seated at one table 410, there is a possibility that some of the users has a dietary restriction. In this case, it should be avoided that a dish ordered by a user who does not have dietary restrictions is mistakenly served to the user having the dietary restriction. This is because the user with the dietary restriction may feel uncomfortable when a dish containing an ingredient to be avoided by the user is erroneously served to the user. Furthermore, if the user with dietary restriction eats the food served by mistake, the improvement of the disease of the user is hindered and the health of the user may be impaired.

In order to avoid such mistakes, it is necessary to have an appropriate mechanism for associating the user with the dish ordered by the user. However, there are currently limited solutions to avoid such mistakes. In particular, no solution is known that can be applied to general restaurant stores such as those shown in FIG. 5. Currently, in a small store where a counter numbered for each seat is installed, there is an attempt of manually associating a user with a dish ordered by the user by using an order slip or using an order input terminal. However, the manual associating operation can cause a dish to be served to a wrong user.

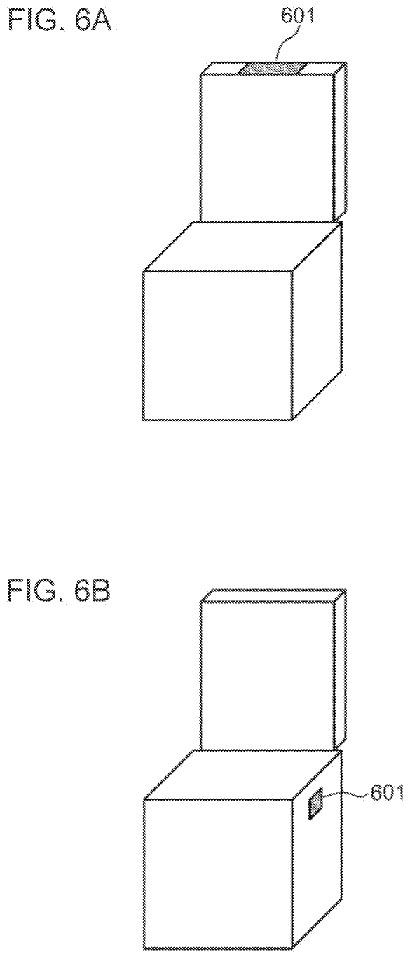

The present embodiment provides a mechanism for more reliably associating and managing a user and a dish ordered by the user. A specific example for realizing this mechanism is described below. In the present embodiment, a QR code is set at each seat in the store 40. FIGS. 6A, 6B, 6C, and 6D each show an example of a manner of setting the QR code 601 on a seat. In the example shown in FIG. 6A, the QR code 601 is set on an upper surface of a backrest of each chair in the store. As described above, the QR code 601 includes the restaurant ID and the seat ID of the seat on which the QR code 601 is set. In this example, the QR code is used, but this is merely an example. Alternatively, any other information such as a bar code may be used as long as the restaurant ID and the seat ID can be identified.

In the example in FIG. 6B, the QR code 601 is set on a side surface of a seat of each chair. By setting the QR code 601 on the side surface of the seat, it becomes possible for the user to easily perform an operation to read the QR code 601.

In the example in FIG. 6C, the QR code 601 is set not on the chair but on a side surface of a table (for example, on a side face facing a chair). In this example, the table is for four people, and four QR codes 601 corresponding to respective four seats are placed on side faces of the table.

In the example in FIG. 6D, the QR code 601 is located on an upper surface of a table top plate. In this example, the table is for four people, and four QR codes 601 corresponding to respective seats are set on the upper surface of the table top plate. By locating the QR codes 601 on the upper surface of the table top plate, it becomes possible for users to easily notice the existence of the QR codes 601.

The QR code prepared for each seat is used by the information terminal 100 to acquire menu information available at the store when a user orders a dish. A detailed description is given below as to a method of ordering a dish using the QR code and the information terminal 100. In the present embodiment, a dish may be ordered using a standard menu or a personalized menu.

Note that in the examples shown in FIGS. 6A to 6D, the QR code 601 is disposed on each seat, but in the mode in which the restaurant ID and the seat ID are acquired by NFC, the IC chip having an NFC communication function is used instead of the QR code 601.

### Ordering Using Standard Menu

The standard menu is used to order a dish by a user who does not have a dietary restriction. The standard menu includes common dishes served for users in the store. A process of ordering a dish via the standard menu is described below with reference to various screens displayed on the information terminal 100.

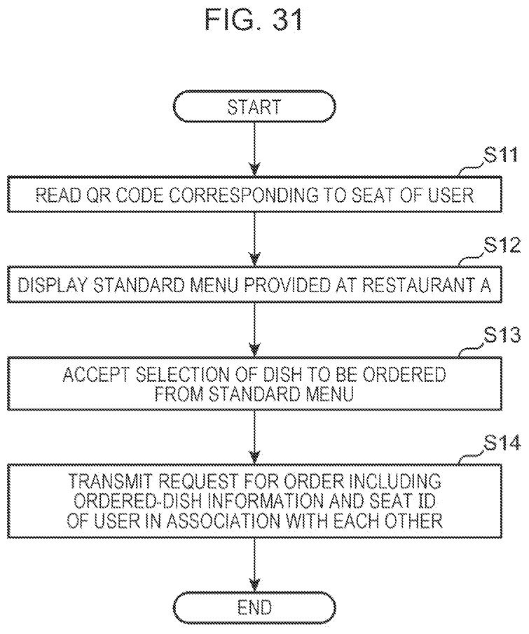

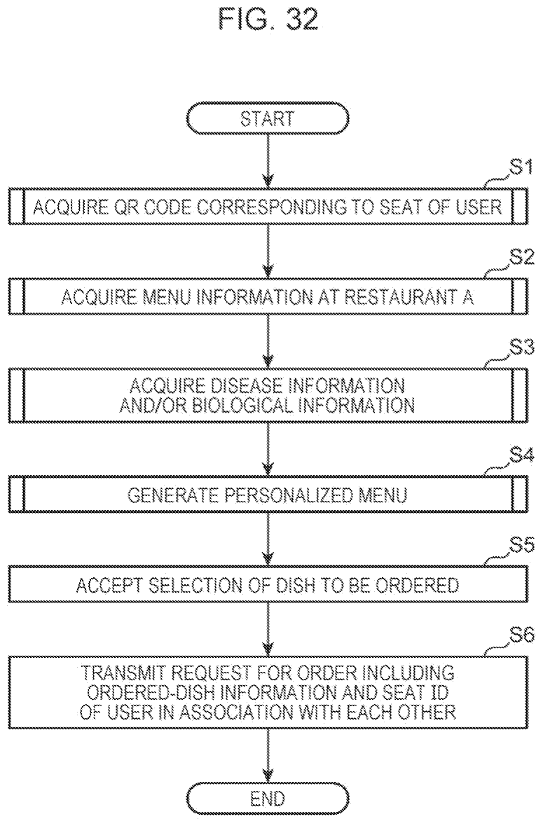

The order process via the standard menu corresponds to the flowchart shown in FIG. 31, and thus the process is described below while referring to the flowchart in FIG. 31. First, the QR code reader is activated, and the QR code reader reads a QR code corresponding to a seat where a user is seated. This process corresponds to step S11 in FIG. 32.

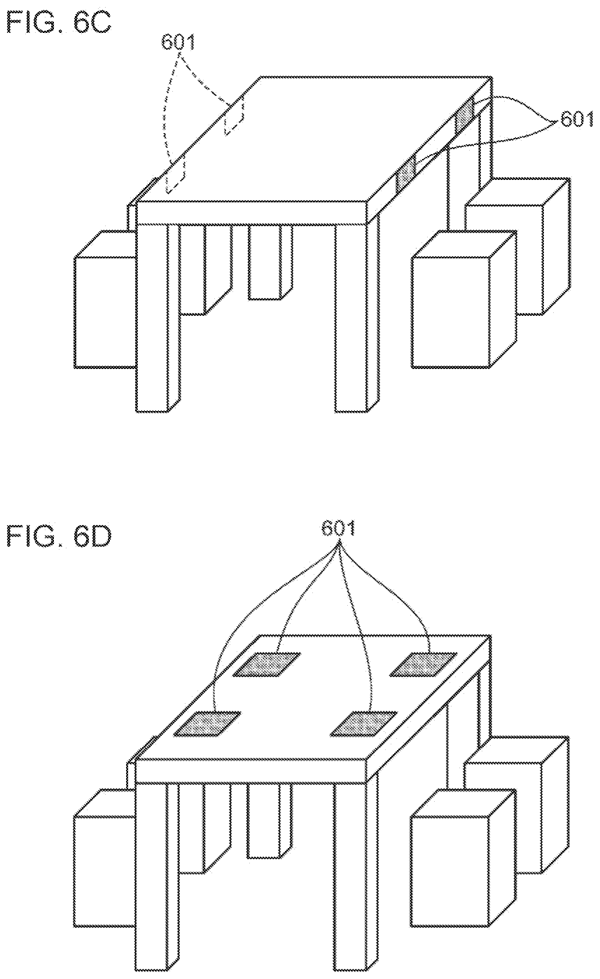

FIG. 7 is a diagram showing an example of an operation screen G1 displayed on the information terminal 100 when the user operates the information terminal 100 to read the QR code. The operation screen G1 is displayed in a situation in which, after the user enters a store of a restaurant and is seated, the user operates the information terminal 100 to read the QR code corresponding to the seat where the user is seated. The QR code corresponding to the seat of the user is arranged in one of the manners shown in FIG. 6A to 6D. After the user is seated, the user takes out the information terminal 100 and operates the information terminal 100 to read the QR code corresponding to the seat of the user so as to acquire the standard menu of the store of the restaurant. The reading of the QR code is realized by using a general-purpose QR code reading application called a “QR code reader” pre-installed on the information terminal 100. FIG. 7 illustrates the operation screen G1 in a situation in which the user is performing an operation of focusing the information terminal 100 on the QR code corresponding to the seat of the user. The user adjusts the orientation and the position of the information terminal 100 such that the QR code 601 fits within an area surrounded by a guide line 701 (a broken-line square in the figure) of the QR code reader. Near the QR code 601 located at each seat, text information “Seat number 18” is provided such that a user or a staff of the store can identify the seat from this information. Furthermore, text information is also provided to indicate what the QR code 601 is (in this specific example, the text information indicating “QR code for personal matching”). Therefore, an image of “Seat number 18” and an image of the “QR code for personal matching” are also displayed on the operation screen G1.

Next, the menu information provided by the restaurant company A is acquired from a QR code read by a QR code reader. A standard menu is generated based on the menu information and displayed on the information terminal 100.

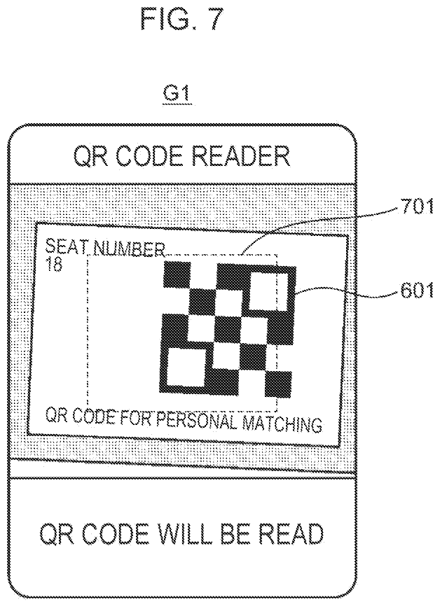

This process corresponds to step S12 in FIG. 31. FIG. 8 is a diagram showing an example of an operation screen G2 which is displayed on the information terminal 100 immediately after the QR code is read by the QR code reader. On this operation screen G2, a character string successfully obtained as a result of reading the QR code by the QR code reader is displayed. In this specific example, “http://restaurantA.com/QRorder-18” obtained as the result of the reading by the QR code is displayed. The operation screen G2 includes a button 801 labeled “OPEN ON BROWSER” and a button 802 labeled “SEND MAIL”. The button 801 is selected when the user recognizes that the character string displayed as the result of reading the QR code represents a URL. When the button 801 is touched, the Internet browser is started, and a web page indicated by this URL is displayed on the information terminal 100.

The button 802 is selected when the user recognizes that the character string displayed as the result of reading the QR code represents a mail address. When the button 802 is touched, a mail application is started. In this specific example, the button 801 is touched to browse the standard menu.

To order a dish from this standard menu, it is not necessary to install a particular application on the information terminal 100, and it is sufficient if the QR code reader and the browser are available. Therefore, it is possible for many users to order dishes using the standard menu.

The browser can identify that the connection destination is the restaurant company A, that is, the restaurant ID, from connection destination information (for example, a domain name (restaurantA.com)) included in the character string (for example, the URL) read by the QR code reader. The second server 300 recognizes that the number at the end of the requested URL is 18, and thus the second server 300 determines that this request is issued from the browser of the information terminal 100, which has read the QR code of the seat with the seat number of “18”. Although the character string shown in FIG. 8 does not include the store ID of the store 40, the character string may explicitly include information indicating the “store 40” (Store-A). In this case, the QR code represents the character string, for example, as “http://restaurantA.com/Store-A/QRorder-18”. The browser of the information terminal 100 acquires the requested connection destination information in the manner described above.

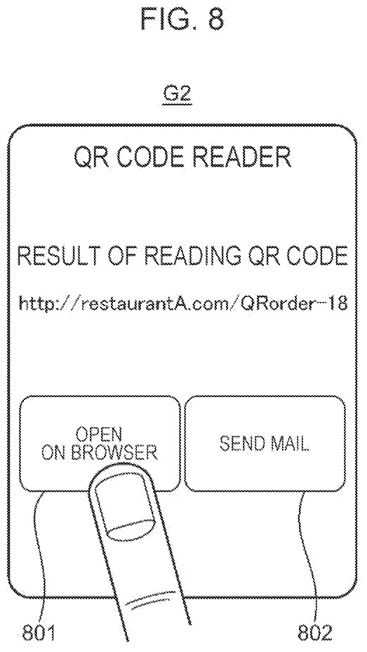

FIG. 9 is a diagram illustrating an example of an operation screen G3 including the standard menu provided by the restaurant company A. This operation screen G3 is displayed when the menu information is received by the information terminal 100. The standard menu includes dishes included in the menu information as they are, and nothing is taken into account as to the information indicating the degree of the dietary restriction of the user. The menu information may be the same as the menu information used in generating a personalized menu, or may be different from it.

A plurality of tile objects 901 are arranged in the form of a matrix on the operation screen G3. The standard menu is formed using these tile objects 901. One tile object 901 corresponds to one dish included in the standard menu. Each tile object 901 includes the name of a dish, the price of the dish, and the image of the dish. On the operation screen G3, the standard menu scrolls in response to a scroll operation performed by the user. As a result, other dishes that are not displayed previously on the operation screen G3 appear on the operation screen G3. By performing the scroll operation in the above-described manner, the user can browse all dishes included in the standard menu.

The operation screen G3 is displayed when the browser connects to the URL indicated by the character string obtained as a result of reading the QR code (for example, http://restaurantA.com/QRorder-18) and the browser receives the menu information from the second server 300 of the restaurant company A.

For example, the browser of the information terminal 100 connects to the above-described URL and issues an HTTP request for an HTML file for drawing the standard menu of the restaurant company A. When the browser receives an HTTP response from the second server 300 of the restaurant company A, the browser draws the operation screen G3 including the standard menu according to the received HTTP response. However, the above-described implementation is merely an example, and the drawing of the operation screen G3 may be realized by other technical means.

Next, the user selects a dish from the displayed standard menu. This process corresponds to step S13 in FIG. 31.

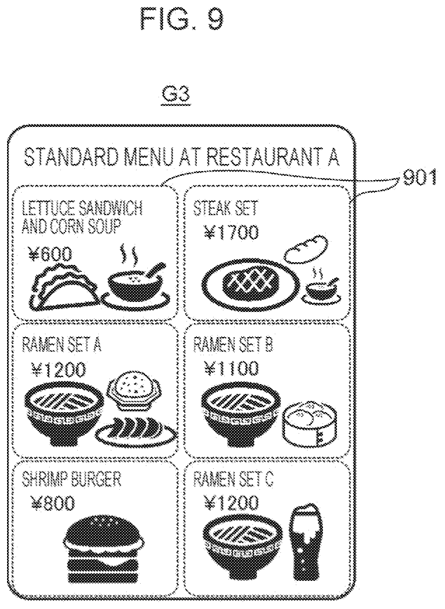

FIG. 10 is a diagram showing an example of a scene in which a user operates the operation screen G3 to order a dish from the standard menu. As shown in this figure, the user can determine a dish to be ordered by performing a touch operation using a pointing element 1001 such as a finger. For example, when the information terminal 100 detects that a tile object 901A corresponding to a dish of “Ramen set B” is touched once, the information terminal 100 changes the color of the tile object 901A from a first color, which is a default color, to a second color to indicate that the tile object 901A is selected. Furthermore, the information terminal 100 displays “1”, for example, in the upper right corner of the tile object 901A thereby indicating the number of ordered dishes for “Ramen set B”. Thus, in this example, the operation screen G3 indicates that the dish of “Ramen set B” is selected from the standard menu. As described above, the user can order a dish by touching a tile object 901 corresponding to a desired dish, that is, the user can intuitively and easily order the desired dish by performing a familiar operation.

In this example, the color of a tile object 901 is changed when the user selects a dish to order, but this is merely an example, and the present embodiment is not limited to this. For example, the pattern of a tile object 901 may be changed from a first pattern to a second pattern when selected by the user. Alternatively, the color and pattern of a tile object 901 may be changed from the first color and the first pattern to the second color and the second pattern when selected by the user.

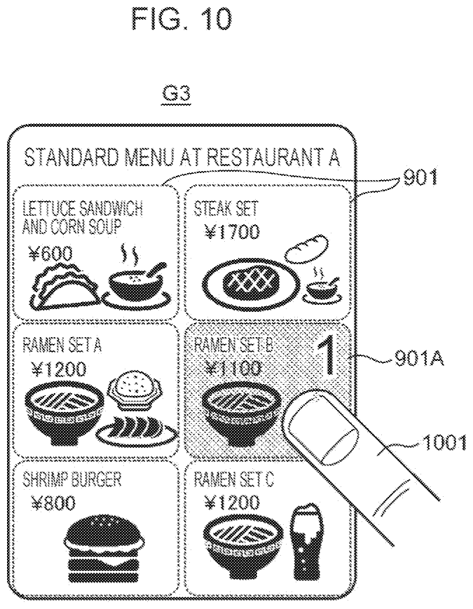

FIG. 11 is a diagram showing an example of an operation screen G4 which is displayed when a dish selected from the standard menu is finally ordered. The operation screen G4 is displayed when an order button (not shown in FIG. 9) on the operation screen G3 is touched by the user to finally decide the order of the dish. The operation screen G4 includes the tile object 901A corresponding to the dish selected on the operation screen G3, a total amount field 1011 in which the total amount of the ordered dishes (for example, 1,100 yen) is displayed, and an order button 1012 which is touched to make a final decision. As described above, the operation screen G4 displays a list of dishes to be ordered, the quantity of each dish, and the total amount of dishes to be ordered such that the user can efficiently confirm the details of the order on a single screen. If the user confirms that the order contents are correct, the user touches the order button 1012 at the bottom of the operation screen G4. As a result, the dish order is finally confirmed. Since the seat number “18” is displayed on the order button, the user can confirm that the ordered dish is to be served to the seat of the user. When the order button 1012 is touched, the information terminal 100 transmits an order request including ordered-dish information indicating the selected dish and the seat ID (“18” in the example shown in FIG. 11) read from the QR code in association with each other, to the second server 300 of the restaurant company A. Thus, the order process using the standard menu is completed. This process corresponds to step S14 in FIG. 31.

The order process using the standard menu for general users is carried out as described above. In this order process, a user can order a dish such that the user operates the information terminal 100 so as to read a QR code and display the standard menu provided at the restaurant company A on the browser, and the user orders the dish via this standard menu. Thus, it is not necessary perform a troublesome operation to pre-install, on the information terminal 100, a specific application such as that provided by the restaurant company A. That is, users can immediately use this service by using the information terminal 100, which makes it possible for many users to use this service. In addition, users can easily select and order a desired dish by intuitive operation through the standard menu. Furthermore, the operation screen G3 allows it to change the zoom magnification by performing a pinching operation. This allows even users with presbyopia to easily see dishes included in the operation menu. Furthermore, users can browse more information at the same time by reducing the operation screen G3. Furthermore, the order request is issued by an HTTP request in which the ordered-dish information and the seat ID (for example, the seat number “18”) are indicated in association with each other, the second server 300 of the restaurant company A recognizes the dish ordered by a user at the seat with the seat number “18” via the HTTP request, and displays the recognized seat number “18” and the ordered dish on a display in the store.

This allows a restaurant employee to serve the ordered dish correctly to the user at the seat with the seat number “18”. Furthermore, the standard menu is provided not by a paper medium. This allows the restaurant company A to avoid a troublesome operation of updating or managing the standard menu, which would be required when the standard menu is provided by a paper medium. As a result, human resources for taking orders and the risk of complaints that mistakenly accept orders are reduced, and cost reduction and management efficiency are achieved.

### Ordering Using Personalized Menu

Next, ordering a dish using a personalized menu is described below. The standard menu is a menu provided by a restaurant for general users. In contrast, the personalized menu is a menu which is for a user having a dietary restriction and includes dishes prepared according to information indicating the degree of dietary restriction of the user. A process of ordering a dish using the personalized menu is shown in a flowchart in FIG. 32. A process of ordering a dish from a personalized menu is described below with reference to the flowchart shown in FIG. 32.

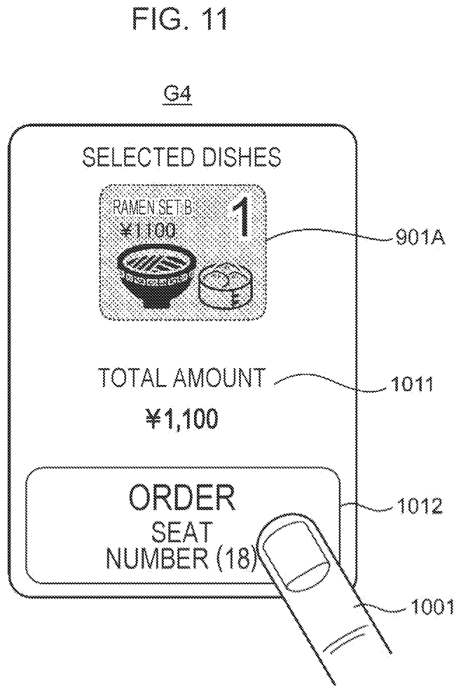

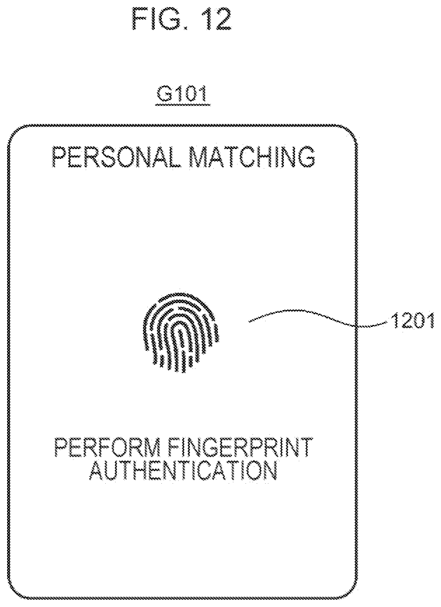

Starting of the process of ordering a dish from the personalized menu is triggered by activating a matching application. FIG. 12 is a diagram showing an example of an authentication screen G101 which is displayed on the information terminal 100 immediately after the matching application is activated by a user who wants to order a dish. The authentication screen G101 is a screen for use by a user in performing a user authentication process using a fingerprint. On the authentication screen G101, a fingerprint image 1201 schematically illustrating a fingerprint is displayed in the center, and a message “Perform fingerprint authenticate” is displayed below the fingerprint image 1201. Thus, the authentication screen G101 prompts the user to perform fingerprint authentication. A character string “Personal matching” is displayed at the top of the authentication screen G101. This allows the user to recognize that the authentication screen G101 is for the matching application. This also applies to FIGS. 13 to 17 described later.

FIG. 13 is a diagram showing another example of an authentication screen G102. The authentication screen G102 is an example of a screen for performing user authentication by face authentication. On the authentication screen G102, a dashed line 1301 schematically showing an outline of a face which is displayed in the center such that the information terminal 100 can capture an image of a face of a user from the front of the user in an appropriate size. The user adjusts the orientation and the position of the information terminal 100 such that the image of the face of the user as captured from the front of the user is displayed within an area surrounded by the dashed line 1301.

In a case where there is a user authentication method that allows a user to perform authentication with sufficiently high accuracy with a less burden on the user as compared with the above-described user authentication methods, such a method may be used. As a method of user authentication, a method of inputting a user ID and a password may be used, or two-step authentication may be employed to achieve a high security strength.

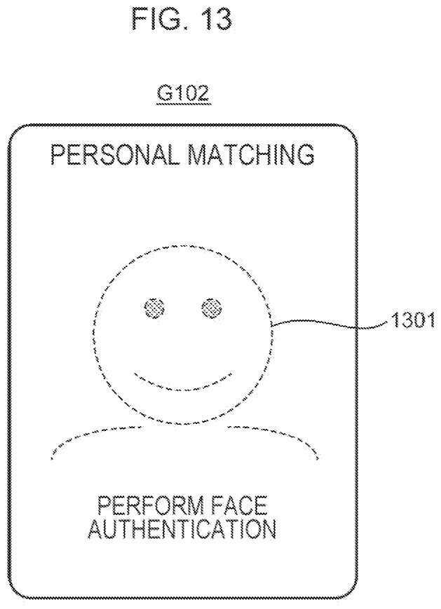

FIG. 14 is a diagram showing an example of a home screen G103 which is displayed immediately after the user authentication by the matching application is completed. On the home screen G103, an application name “PERSONAL MATCHING” is displayed in a top area, and a plurality of tile objects 1401 are displayed in the form of a matrix in a middle area. Each tile object 1401 is associated with a cooperation function or another application incorporated by the matching application. Another application is, for example, an application which runs in the matching application. In this example, five tile objects 1401 labeled a, b, c, d, and e are displayed. These tile objects 1401 are respectively associated with dedicated functions (for example, an application in the matching application) that perform matching with company's products or services in cooperation with the matching application. Thus, the user can use five cooperation functions indicated by a, b, c, d, and e. Grayed-out tile objects 1401 are empty tile objects to which no cooperation function is not installed. At the bottom of the home screen, from the left to the right, a scan button 1402, a map button 1403, an account button 1404, and a home button 1405 are displayed. These four buttons are fixed buttons. The scan button 1402 is a button used when reading a QR code or the like linked to a service provided by a company such as a restaurant or the like described above. The map button 1403 is a button used to display, on a map screen, stores which are located near the current location of the information terminal 100 and in which the matching application is usable. The account button 1404 is a button for registering or editing user's account information. Registration and editing of the account information includes, for example, setting of personal authentication and setting of a cooperation function with the first server 200. The home button 1405 is a button used to return the display screen to the home screen G103 shown in FIG. 14.

On the home screen G103, tile objects 1401 for using a cooperation function, another application, and a service provided by another company are collectively arranged in the middle area. These tile objects 1401 can be set according to preference of a user in terms of whether each tile object 1401 is displayed or not, and where each tile object 1401 is displayed. Therefore, the user is allowed to acquire, by using a single matching application, products and/or services suitable for the user selected based on personal information from those provided by many companies (for example, home appliance mass retailers, DVD/Blu-ray (registered trademark) rental stores, bookstores, coffee shops, taxis, etc.).

FIG. 15 is a diagram showing an example of an operation screen G104 which is displayed on the information terminal 100 when, after the user starts the matching application, the user operates the information terminal 100 to read a QR code corresponding to a seat of the user. The operation screen G104 (an example of a first operation screen) is similar to the operation screen G1 shown in FIG. 7 except that in the operation screen G104, the name of the application displayed on the top is “PERSONAL MATCHING”.

The matching application can determine that a connection destination is the restaurant company A from a connection destination information (for example, a domain name, restaurantA.com) indicated by a character string (for example, a URL) read from the QR code. When the second server 300 receives this request, the second server 300 detects that the number at the end of the URL requested by the matching application is “18”, and thus the second server 300 determines that this request is a request transmitted from the information terminal 100 that read the QR code of the seat with the seat number “18”. In the present embodiment, it is assumed by way of example that a user seated in a seat with the seat number “18” in a store 40 of the restaurant company A orders a dish. In this request, the matching application may explicitly specify the store 40 (Store-A). In this case, the character string represented by the QR code may be set as, for example, http://restaurantA.com/Store-A/QRorder-18. The matching application can acquire information identifying the connection destination (for example, the restaurant ID) in the manner described above.

The above-described process of starting the matching application, performing user authentication, and reading the QR code corresponds to step S1 in FIG. 32.

In the mode in which a restaurant ID and a seat ID are acquired using NFC instead of using a QR code, a first operation screen described below is used. FIG. 16 is a diagram showing an example of an operation screen G1011 which is displayed on the information terminal 100 when a restaurant ID and a seat ID are acquired via NFC. As with the operation screen G104, this operation screen G1011 is also displayed when the scan button 1402 is touched on the home screen G103.

A character string “PERSONAL MATCHING” is displayed at the top of the operation screen G1011 to indicate that this screen is for the matching application. In the center of the screen, there are displayed an NFC mark 1601 that symbolically represents NFC and a message prompting a user to bring the information terminal 100 closer to an object with the NFC mark 1601 (for example, “BRING THIS DEVICE CLOSE TO NFC MARK”).

In the case where a restaurant ID and a seat ID are acquired via NFC, an IC chip, which includes a memory storing the restaurant ID and the seat ID and which has an NFC communication function, is disposed on each seat of a restaurant. The NFC mark 1601 may be displayed on this IC chip. This makes it possible for a user to detect the NFC mark 1601 on the operation screen G1011 and easily understand that the user is requested to bring the information terminal 100 close to the IC chip disposed on the seat of the user. As with the QR code 601 described above, the information stored in memory of the IC chip may be URL of the restaurant such that the restaurant ID and the seat ID can be identified by the URL.

Next, a description is given below as to a process performed by the matching application to access the second server 300 of the restaurant company A and acquire menu information. This process corresponds to step S2 in FIG. 32.

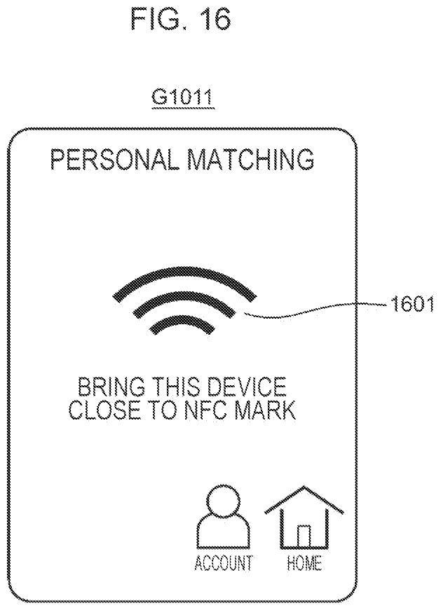

FIG. 17 is a diagram showing an example of a display screen G105 which is displayed on the information terminal 100 when the matching application is generating a personalized menu. A rotating circular arrow object 1501 is displayed on the display screen G105. Furthermore, below the arrow object 1501, a character string “MATCHING IS BEING PERFORMED WITH MENU AT RESTAURANT A” is displayed. This allows the user to recognize that the matching application is in operation.

When the display screen G105 is being displayed, the matching application on the information terminal 100 generates a personalized menu in cooperation with the second server 300 of the restaurant company A and the first server 200. More specifically, the matching application accesses the second server 300 of the restaurant company A based on the URL read from via the QR code 601 or via NFC and acquires the menu information. After the matching application acquires the menu information, the matching application detects a data attribute of the menu information. In this case, the menu information is information regarding food, and thus a food attribute is detected as the data attribute.

The menu information is given, for example, in the form of an HTML file. For example, the menu information includes information indicating that the data attribute is the food attribute described in a predetermined format. According to this format, the matching application may detect that the data attribute of the menu information is the food attribute. Alternatively, the matching application may detect that the data attribute of the menu information is the food attribute from a domain name of the URL indicated by the QR code. In this specific example, the domain name “restaurantA.com” indicates the restaurant company A, and thus it is determined that the data attribute of the menu information is the food attribute. Alternatively, the matching application may analyze the acquired menu information, and may detect the data attribute such that in a case where the analysis indicates that the data is related to food, it is determined that the data attribute of the menu information is the food attribute. Alternatively, the matching application may detect the data attribute such that supplementary information indicating the data attribute of the menu information is acquired from the second server 300, and it is determined that the data attribute of the menu information is the food attribute. Another method may be used in the implementation of the process of detecting the data attribute of the menu information as long as the data attribute can be identified.

Next, a description is given below as to a process performed by the matching application to acquire disease information and/or biological information from the second server 300. This process corresponds to step S3 in FIG. 32.

When the matching application recognizes that the data attribute of the menu information is the food attribute, the matching application requests the first server 200 to provide latest disease information and/or biological information classified in the food attribute. This request includes the user ID. Upon receiving this request, the first server 200 extracts, based on the user ID, the latest disease information and/or biological information from the personal information which is distributed and encrypted. The extracted disease information and/or biological information is transmitted from the first server 200 to the information terminal 100. Thus, the matching application acquires the disease information and/or biological information.

The information terminal 100 generates information indicating the degree of dietary restriction of the user based on the acquired disease information and/or biological information. After the information terminal 100 generates the information indicating the degree of dietary restriction, the information terminal 100 checks the menu information provided by the restaurant company A with reference to the information indicating the degree of dietary restriction, and generates a personalized menu. This process corresponds to step S4 in FIG. 32. During this process, the display screen G105 shown in FIG. 17 is still displayed on the information terminal 100, although the matching application is executing the process of checking the menu information with respect to the information indicating the degree of dietary restriction and generating the personalized menu according to the information indicating the degree of dietary restriction.

There are a plurality of variations of the personalized menu generated.

In a first variation, the computational processing unit 104 generates a personalized menu based on the menu information and the information indicating the degree of dietary restriction such that when a dish included in the menu information includes an ingredient that is specified as an ingredient to be avoided by the user by the information indicating the degree of dietary restriction, the amount of the ingredient contained in the dish is reduced and the resultant dish is incorporated into the personalized menu.

For example, for a user suffering from gout, ingredients including purine, such as shrimp or liver, should be avoided. In this case, if the menu information includes a dish including an ingredient including purine, the amount of the ingredient including purine for this dish is reduced to be less than the maximum acceptable intake thereof. The reducing of the amount of ingredient includes completely removing the ingredient such that the ingredient is not included at all. For example, in a case where the personalized menu for the user suffering from gout is generated based on the menu information, if the menu information includes a dish including an ingredient including purine, such as shrimp or liver, the amount of the ingredient included in such a dish is reduced to zero.

Since the ingredient information 2800 includes information indicating the amounts of ingredients included in each dish, the computational processing unit 104 can determine, for each dish, whether or not the dish includes an ingredient to be avoided by the user and can identify the amount of ingredient included in the dish. Then, the personalized menu may be generated such that if there is a dish including an ingredient specified as an ingredient to be avoided by the ingredient information 2800, the amount of the ingredient is reduced to be less than the maximum acceptable intake or is removed to zero.

In a second variation, the computational processing unit 104 generates a personalized menu based on the menu information and the information indicating the degree of dietary restriction such that when the menu information includes a dish containing an ingredient specified as an ingredient to be avoided by the user by the information indicating the degree of dietary restriction, the dish is excluded from the personalized menu or grayed out on the personalized menu.

Let it be assumed here by way of example that shrimp is the ingredient that the user should avoid, and the menu information includes shrimp hamburg steak. In this case, the tile object 901 indicating the shrimp hamburg steak is excluded from the personalized menu or grayed out on the personalized menu. Graying out is a display mode in which the tile object 901 of the shrimp hamburg steak is displayed semi-transparently, for example, in a gray color.

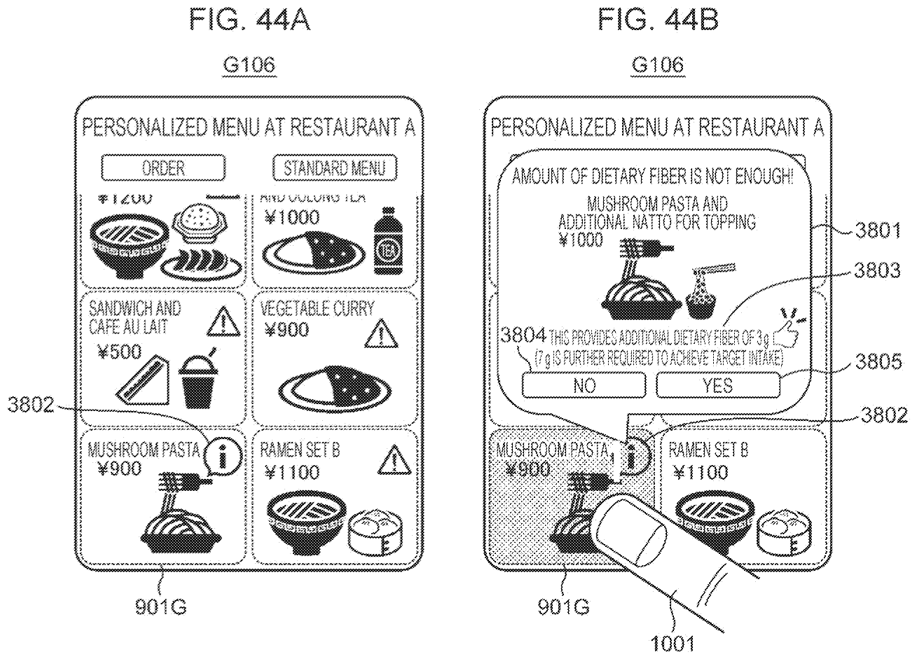

In a third variation, the computational processing unit 104 generates a personalized menu such that an ingredient containing a nutrient that tends to be deficient depending on the progress of the disease of the user is added to a dish in the personalized menu. For example, the computational processing unit 104 may identify a nutrient which tends to be deficient depending on the progress of the disease of the user based on the disease information and/or biological information, and may add an ingredient containing the nutrient to a dish in the personalized menu. For example, let it be assumed here by way of example that dietary fiber is identified as a nutrient that tends to be deficient from the biological information and/or disease information for a user. In this case, a dish including much dietary fiber (for example, natto) is added to the personalized menu. In a specific example, as will be described later with reference to FIGS. 44A and 44B, when a user selects a certain dish from a personalized menu, the display 105 displays a screen prompting the user to order a dish containing a nutrient that tends to be deficient in addition to the selected dish. In this case, a dish containing a nutrient that tends to be deficient may be determined in advance for each dish, and the computational processing unit 104 may select such a dish corresponding to the dish selected by the user and may add it to the personalized menu.

In a fourth variation, the computational processing unit 104 generates a personalized menu including a description of a combination of dishes for supplementing a nutrient that tends to be deficient depending on the progress of the disease of the user. For example, let it be assumed here by way of example as in the third variation that dietary fiber is identified as a nutrient that tends to be deficient depending on the progress of the disease of the user from the disease information and/or biological information of the user. In this case, for example, the personalized menu includes a set of dishes including natto.

In a fifth variation, the computational processing unit 104 generates a personalized menu including a dish obtained by adding, to a dish included in the one or more dishes, an ingredient including a nutrient lacking in a past meal of the user in a particular period. The predetermined period is a period with a length of 1 day, 2 days, 5 days, 1 week, or 1 month. For example, the computational processing unit 104 may acquire the dish order history information in the predetermined period from the first server 200, and may identify the nutrient deficient for the user from the acquired order history information. For example, the order history information includes information about dishes ordered by the user in the past. The information about dishes includes, in addition to the names of the dishes, for example, the ingredients used in each dish and their amounts. If the ingredient and the amount thereof are known, the nutrients included in each ingredient and the amount thereof can be determined. Therefore, the computational processing unit 104 identifies the nutrients taken by the user and the amount of each nutrient for each dish included in the order history information, and calculates the cumulative value of the amount of each identified nutrient in the predetermined period. Thereafter, the computational processing unit 104 determines whether or not the cumulative value is smaller than a predetermined threshold value for each nutrient, detects nutrients whose cumulative values are smaller than the corresponding threshold values, and determines the detected nutrients as deficient nutrients. For example, in a case where it is determined that dietary fiber is a deficient nutrient, a dish including much dietary fiber (for example, natto) is added to the personalized menu.

In a sixth variation, the computational processing unit 104 generates a personalized menu including a combination of dishes to supplement a nutrient deficient in past one or more meals taken by a user in a predetermined period. The predetermined period may be similar to that in the fifth variation. The computational processing unit 104 may identify a deficient nutrient by using the method described in the fifth variation. For example, in a case where it is determined that dietary fiber is a deficient nutrient, the personalized menu includes a set of dishes including a dish containing much dietary fiber (for example, natto).

In a seventh variation, the information indicating the degree of dietary restriction includes information indicating the maximum acceptable intake in a current meal for a specific nutrient related to the disease of the user. The computational processing unit 104 generates a personalized menu such that a dish including a greater amount of specific nutrient than the maximum acceptable intake in a current meal is detected from dishes included in the menu information, and the amount of specific nutrient in the dish is reduced to be equal to or smaller than the maximum acceptable intake in the current meal and the resultant dish is incorporated into the personalized menu.

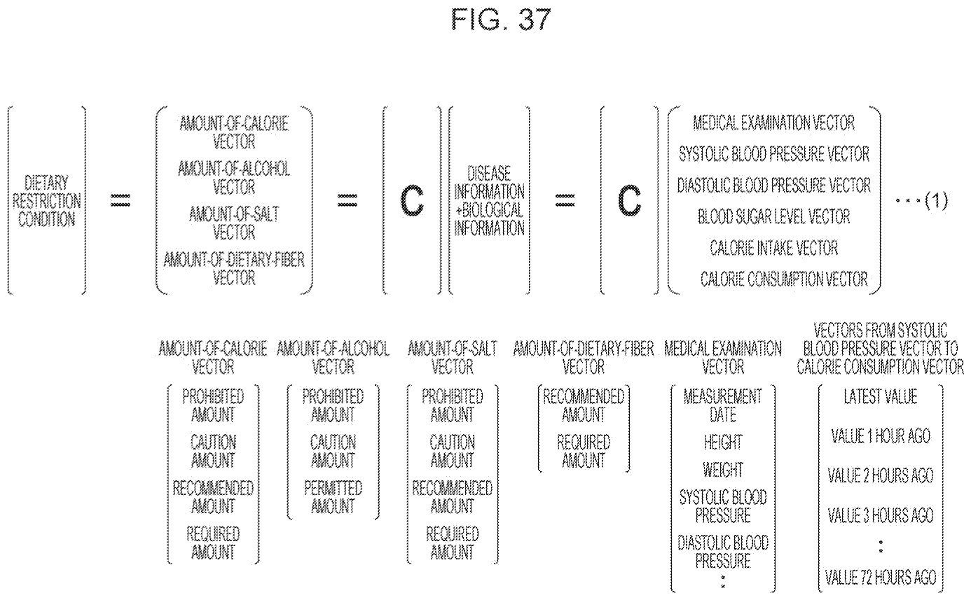

The maximum acceptable intake in the current meal is given by a value obtained by subtracting the cumulative intake in the past meals in the particular period from the maximum acceptable intake in the particular period, wherein a predetermined margin may be further subtracted. The particular period may be period with a length of 1 day, 2 days, 5 days, 1 week, 1 month, etc., and there are no particular restrictions. This also applies to the following variations. For diabetic patients, for example, alcohol is specified as an ingredient to be avoided, and thus alcohol is a specific nutrient. The maximum acceptable intake of alcohol in the particular period can be calculated, for example, by using equation (1) described later with reference to FIG. 37. The cumulative intake of alcohol in the past meals in the particular period can be calculated based on the biological information and/or the dish order history information. The computational processing unit 104 may generate a personalized menu based on the menu information such that when a dish included in the menu information contains a greater amount of alcohol than the maximum acceptable intake in the current meal, the ingredient information 2800 about this dish is changed such that the amount of alcohol is reduced to be equal to or less than the maximum acceptable intake in the current meal or is reduced to zero, and the dish on the personalized menu is modified according to the modified ingredient information 2800.

In an eighth variation, the information indicating the degree of dietary restriction includes information indicating the target intake in the current meal for a specific nutrient related to the disease of the user. The computational processing unit 104 generates a personalized menu such that when a dish in the one or more dishes included in the menu information includes a smaller amount of specific nutrient than the target intake in the current meal, the amount of specific nutrient of this dish is increased to be equal to or greater than the target intake in the current meal, and the resultant dish is incorporated into the personalized menu.

The target intake in the current meal is given by a value obtained by subtracting the cumulative intake in the past meals in the particular period from the target intake in the particular period wherein a predetermined margin may be further subtracted. For diabetic patients, for example, an intake of dietary fiber is recommended, and thus dietary fiber may be specified as the specific nutrient. The target intake of dietary fiber in the particular period can be calculated using equation (1) described later with reference to FIG. 37. The cumulative intake of dietary fiber in the past meals in the particular period can be calculated based on the biological information and/or the dish order history information. The computational processing unit 104 may generate a personalized menu based on the menu information such that when a dish in the menu information includes a smaller amount of dietary fiber than the target intake in the current meal, the ingredient information 2800 related to this dish is modified such that the amount of dietary fiber is equal to or greater than the target intake in the current meal, and the dish on the personalized menu is modified according to the modified ingredient information 2800.

In a ninth variation, the information indicating the degree of dietary restriction includes information indicating the maximum acceptable intake of calories in a current meal depending on a disease of a user. The computational processing unit 104 generates a personalized menu such that when a dish in the one or more dishes includes a greater amount of calories than the maximum acceptable intake of calories in a current meal, the amount of calories of the dish is reduced to be equal to or smaller than the maximum acceptable intake. The maximum acceptable intake of calories in the current meal is given by a value obtained by subtracting the cumulative intake of calories in the past meals in the particular period from the maximum acceptable intake of calories in the particular period and further subtracting a predetermined margin. The maximum acceptable intake of calories in the particular period can be calculated, by using equation (1) described later with reference to FIG. 37. The cumulative intake of calories in the past meals in the particular period can be calculated based on the biological information and/or the dish order history information. The computational processing unit 104 may generate a personalized menu based on the menu information such that when a dish in the menu information includes a greater amount of calories than the maximum acceptable intake of calories in a current meal, the ingredient information 2800 about this dish is modified such that the amount of calories of the dish is reduced to be equal to or smaller than the maximum acceptable intake of calories in the current meal, and the dish in the personalized menu is modified according to the modified ingredient information 2800.

In a 10th variation, the 7th variation is modified such that when a dish in the one or more dishes in the menu information includes a greater amount of specific nutrient than the maximum acceptable intake in the current meal, the dish is excluded from the personalized menu or grayed out on the personalized menu. For example, in a case where any dish in the menu information includes a greater amount of alcohol than the maximum acceptable intake in a current meal, the computational processing unit 104 generates a personalized menu such that such a dish is excluded from the personalized menu or grayed out in the personalized menu. The excluding and the graying out are performed in a similar manner as in the second variation.

In a 11th variation, the 8th variation is modified such that when any dish in the menu information includes a smaller amount of specific nutrient than the target intake in the current meal, such a dish is excluded from the personalized menu or grayed out in the personalized menu. For example, when a dish in the menu information includes a smaller amount of dietary fiber than the target intake in a current meal, the computational processing unit 104 generates a personalized menu such that the dish is excluded from the personalized menu or grayed out on the personalized menu.

In a 12th variation, the 9th variation is modified such that when a dish in the one or more dishes in the menu information includes a greater amount of calories than the maximum acceptable intake in a current meal, the dish is excluded from the personalized menu or grayed out on the personalized menu. For example, in a case where any dish in the menu information includes a greater amount of calories than the maximum acceptable intake in the current meal, the computational processing unit 104 generates the personalized menu such that such a dish is excluded from the personalized menu or grayed out in the personalized menu.

In a 13th variation, the 7th variation is modified such that when a dish in the one or more dishes in the menu information includes a greater amount of nutrient than the maximum acceptable intake in a current meal, the dish is added with an ingredient including a nutrient having an effect that intake of the added nutrient together with the specific nutrient neutralizes adverse effects of the intake of a greater amount of specific nutrient than the maximum acceptable intake. For example, for diabetic patients, restrictions are imposed in terms of the intake of glucide and the intake of calories. Dietary fiber is required to be chewed slowly, and thus dietary fiber can make people with diabetes feel full while chewing, and can reduce calorie intake and prevent a rapid rise in blood sugar level. Therefore, dietary fiber may be the specific nutrient. Examples of ingredients containing much dietary fiber are natto, seaweed, etc. Therefore, in the present variation, a personalized menu is generated such that when a dish in the menu information includes a greater amount of, for example, glucide than the maximum acceptable intake in a current meal, an ingredient such as natto, seaweed, or the like is added to the dish.

In a 14th variation, the 7th variation is modified such that a personalized menu includes a description of a combination of a dish including a greater amount of specific nutrient than the maximum acceptable intake in a current meal and a dish including a neutralizing nutrient having an effect that intake of the neutralizing nutrient together with the specific nutrient neutralizes adverse effects of the intake of a greater amount of specific nutrient than the maximum acceptable intake. In this variation, for example, the personalized menu includes a set of dishes in which natto, seaweed salad, or the like described as examples in the 13th variation is combined with a dish including a greater amount of glucide than the maximum acceptable intake in the current meal.

In a 15th variation, the 7th variation is modified such that the personalized menu includes a description of a combination of dishes providing a total amount of intake equal to or less than the maximum acceptable intake in a current meal. In this variation, for example, a personalized menu for a diabetic patient includes a combination of dishes providing a total amount of glucide equal to or smaller than the maximum acceptable intake in a current meal. For example, in a case where the menu information includes a set of dishes, the computational processing unit 104 arranges the set of dishes such that the total amount of glucide included in the set of dishes is set to be equal to or smaller than the maximum acceptable intake in a current meal. Alternatively, the computational processing unit 104 may extract, from the menu information, a combination of dishes resulting in a total amount of glucide equal to or smaller than the maximum acceptable intake in a current meal, and may incorporate the combination of dishes as a set of dishes into the personalized menu.

In a 16th variation, the 8th variation is modified such that a personalized menu includes a description of a combination of dishes resulting in a total amount of intake equal to or greater than the target intake in a current meal. In this variation, for example, a personalized menu for a diabetic patient includes a combination of dishes resulting in a total amount of dietary fiber equal to or greater than the target intake in a current meal. For example, in a case where the menu information includes sets of dishes, if the total amount of dietary fiber included in a set of dishes is equal to or greater than the target intake in a current meal, the computational processing unit 104 may incorporate this set of dishes into the personalized menu. Alternatively, the computational processing unit 104 may extract, from the menu information, a combination of dishes resulting in a total amount of dietary fiber equal to or greater than the target intake in a current menu, and may incorporate this combination of dishes as a set of dishes into the personalized menu.

In a 17th variation, the 9th variation is modified such that the personalized menu includes a description of a combination of dishes including a total amount of calories equal to or smaller than the maximum acceptable intake of calories in a current meal. In this variation, for example, a personalized menu for a diabetic patient includes a combination of dishes including a total amount of calories equal to or smaller than the maximum acceptable intake of calories in a current meal. For example, in a case where the menu information includes sets of dishes, if the total amount of calories included in a set of dishes is equal to or smaller than the maximum acceptable intake of calories in a current meal, the computational processing unit 104 may incorporate this set of dishes into the personalized menu. Alternatively, the computational processing unit 104 may extract, from the menu information, a combination of dishes resulting in a total amount of calories equal to or smaller than the maximum acceptable intake of calories in a current meal, and may incorporate the extracted combination of dishes as a set of dishes into the personalized menu.

The 17 variations described above may be combined as appropriate.

After the matching application generates the personalized menu, the matching application displays the resultant personalized menu on the information terminal 100 using a browser in a similar manner to the case in which the standard menu for taking an order is displayed. The dishes listed in the personalized menu are all such dishes in which the latest biological information and/or disease information are taken into consideration. This makes it possible for a user to smoothly order a dish.

FIG. 18 is a diagram illustrating an example of an operation screen including a personalized menu. This operation screen G106 (an example of the second operation screen) includes a plurality of tile objects 901 arranged in the form of a matrix as with the operation screen G3. On the operation screen G106, the personalized menu is configured to be scrollable in response to a scroll operation performed by the user. The personalized menu is formed by these tile objects 901. In the personalized menu displayed on the operation screen G106, tile objects representing dishes of “steak set”, “ramen set A”, “ramen set B”, “shrimp burger”, and “ramen set C” included in the standard menu shown in FIG. 9 are replaced with tile objects 901 representing dishes of “low-salt ramen and vegetable dumplings”, “vegetable curry and oolong tea”, “pasta with tomato sauce”, “set meal with grilled fish”, and “soba noodles”. This replacement is performed because the steak set is high in calories, the ramen set A is high in salt and glucide and high in calories, the ramen set B is high in salt and glucide and high in calories, the shrimp burger is high in purines, and the ramen set C is high in salt, glucide, and alcohol. These dishes are displayed also in the standard menu, but in a personalized menu, they are lowered in priority (that is, they are displayed at positions which can be displayed on the display 105 after many scroll operations are performed from the initial display of the personalized menu), they may not be displayed, or they may be displayed in a grayed layout fashion or a disabled fashion.

At the top of the operation screen G106, a character string “PERSONALIZED MENU AT RESTAURANT A” is displayed, which explicitly indicates that the personalized menu displayed on the operation screen G106 is a menu personalized for a specific user. This allows the user to understand that the user can safely eat any dishes listed on the personalized menu displayed on the operation screen G106.

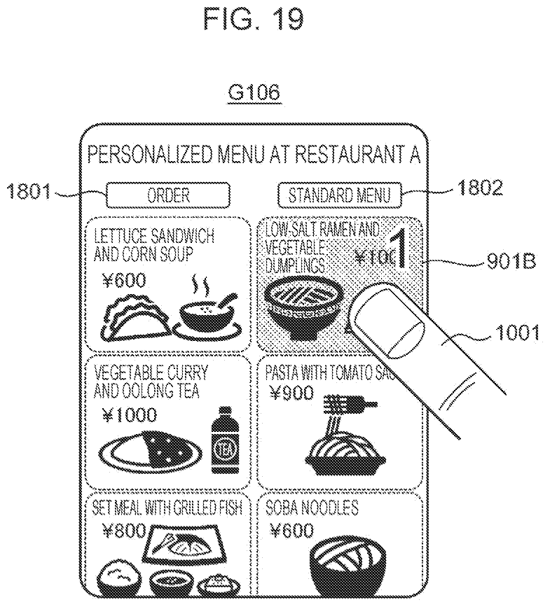

On the operation screen G106, an order switch button 1801 and a standard menu button 1802 are displayed above the tile objects 901. The order switch button 1801 is a button for switching the displayed screen to the operation screen G107 shown in FIG. 20 after selecting a tile object 901. The standard menu button 1802 is a button for switching the displayed screen to the standard menu screen (an example of a general menu) instead of the personalized menu screen. For example, some users with dietary restrictions may not restrict their diet when eating with friends. The standard menu button 1802 is provided to make it possible to meet such a user's demand. When the standard menu button 1802 is touched, the operation screen G106 including the standard menu such as that shown in FIG. 9 is displayed on the display 105.

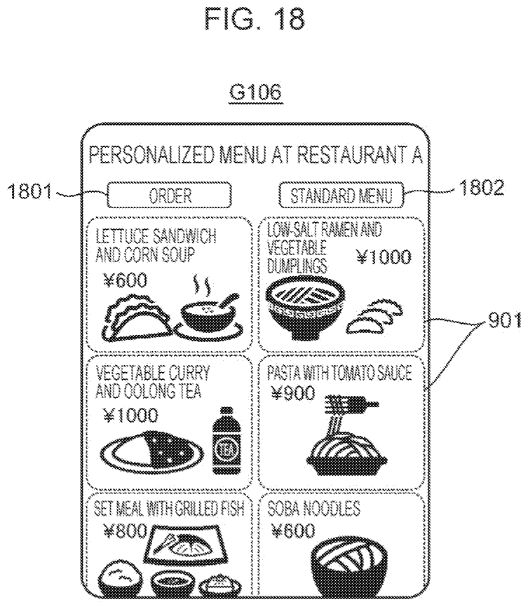

After the matching application displays the personalized menu, the matching application executes a process of accepting a selection of a dish to be ordered by the user. This process corresponds to step S5 in FIG. 32. FIG. 19 is a diagram showing a scene in which a user operates the operation screen G106 to select a dish from a personalized menu and order the selected dish.

In this example of the operation screen G106 shown in FIG. 19, a tile object 901B of the dish of “low-salt ramen and vegetable dumplings” is touched once on the personalized menu. In response, the color of the tile object 901B is changed from a first color to a second color, and “1” indicating the order quantity is displayed in the upper right of the tile object 901B. In this way, the user can easily and intuitively order a dish by performing a touch operation using a pointing element 1001 such as a finger.

In the example described above, the color of the tile object 901 is changed when the user selects a dish to order. However, this is merely an example. For example, the pattern of a tile object 901 may be changed from a first pattern to a second pattern when selected by the user. Alternatively, the color and pattern of the tile object 901 may be changed from the first color and the first pattern to the second color and the second pattern when the tile object 901 is selected by the user.

When the matching application accepts a dish order via the operation screen G106, the matching application sends an order request including the ordered-dish information and the seat ID associated with each other to the second server 300. FIG. 20 is a diagram showing an example of an operation screen G107 which is displayed when a dish is selected from a personalized menu and the selected dish is finally determined to be ordered. This operation screen G107 is displayed when the order switch button 1801 is touched on the operation screen G106.

The operation screen G107 includes the tile object 901B corresponding to the dish selected on the operation screen G106 and the total amount field 2001 indicating the total amount (\1,000) of the ordered dishes (low-salt ramen and vegetable dumplings). The operation screen G107 further includes an order button 2000. The contents of this operation screen G107 are the same as those of the operation screen G4 in the standard menu. When the order button 2000 is touched, an order request including the ordered-dish information and the seat ID associated with each other is sent to the second server 300 of the restaurant company A. The order request is displayed on a display installed in a store of the restaurant company A. Thus, a restaurant staff can grasp the order content from the displayed seat number “18” and the ordered-dish information, and start cooking and serve the ordered dishes to the seat number “18”.

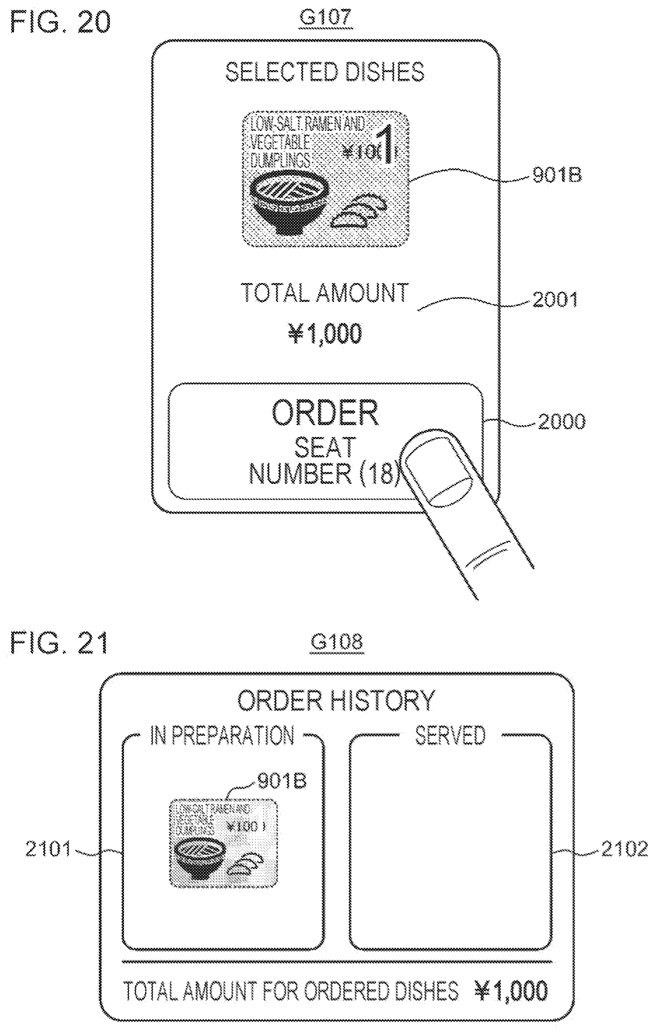

FIG. 21 is a diagram showing an example of an order history screen which is displayed in response to a request for seeing an order history issued by a user. The order history screen G108 includes an in-preparation frame 2101 displayed on the left side of the screen and a served-dish frame 2102 displayed on the right side of the screen. Inside the in-preparation frame 2101, a tile object 901B indicating a dish that has been ordered and is now in preparation is displayed. Inside the served-dish frame 2102, a tile object 901 indicating a served dish is displayed. In the specific example shown in FIG. 20, no dishes have been served yet, and thus no tile objects 901 are displayed in the served-dish frame 2102. Inside the in-preparation frame 2101, tile object 901B indicating the dish of “low-salt ramen and vegetable dumplings” is displayed. On the order history screen G108, the total amount (\1,000) for the dishes ordered already via the matching application is displayed in an easy-to-understand manner at the bottom of the screen. By checking the order history screen G108, the user can see at a glance the dishes ordered so far, the quantity, and the payment amount. An “order history” button (not shown) may be provided on the operation screen G106, and the order history screen G108 may be displayed when the “order history” button is touched.

The processing on the order via the personalized menu at the restaurant company A is performed as described above. When a user wants to order a dish, the user can acquire a personalized menu prepared taking into account the biological information and/or the disease information of the user simply by operating the information terminal 100 to read a QR code assigned to a seat of the user in the restaurant using the matching application provided by the first server 200, and the user can select a dish from the personalized menu to order it. This is an unprecedented, easy, and unmistakable way to order personalized dishes. To make it possible to execute the order process, it is sufficient that the user installs the matching application on the information terminal 100 in advance.

### Dietary Restrictions

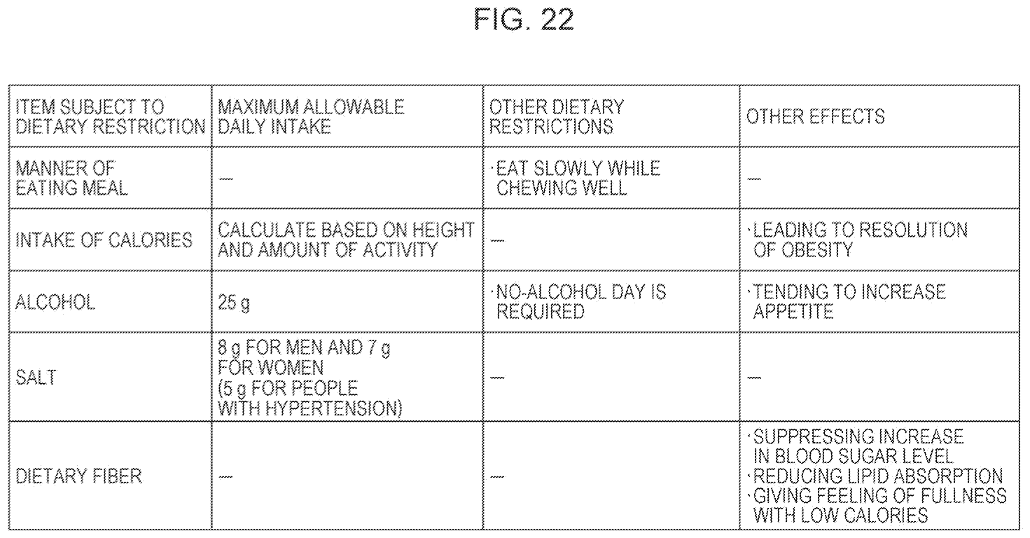

FIG. 22 is a table summarizing examples of dietary restrictions imposed on diabetic patients. First of all, it is recommended to chew slowly and well as a “manner of eating”. Following this recommendation makes it possible to suppress a rapid rise in blood sugar level, which is important for diabetic patients. Furthermore, a feeling of fullness is obtained while chewing slowly and well, which results in a reduction in the intake of calories and glucide.

Diabetic patients are subject to restrictions on maximum acceptable daily intake of calories, salt, and alcohol.

The appropriate daily calorie intake is calculated, for example, as follows.

The appropriate calorie intake [Kcal/day]=standard weight [Kg]*amount of activity [Kcal/day/Kg]=(height [m]*height [m]*22 [Kg])*amount of activity [Kcal/day/Kg]. The amount of activity is set, for example, as follows.

Amount of activity=25 to 30 (mild activity such as desk work), 30 to 35 (moderate activity such as of standing work), or 35 and higher (high activity such as hard work). For example, the proper calorie intake for a user who is 171 cm tall and is a desk work worker on a day with a mild activity is calculated as (1.71*1.71* 22)*(25 to 30)=1608 to 1930 Kcal.

In the present embodiment, the appropriate calorie intake calculated by the above-described method may be employed as the maximum acceptable intake of calories in the particular period, or the maximum acceptable intake of calories in the particular period may be calculated according to equation (1) described later with reference to FIG. 37.

For example, a recommended value of the maximum daily intake of alcohol is equal to or smaller than 25 g/day. In this case, in the present embodiment, the amount of alcohol ingested by drinking alcohol may be displayed on a personalized menu. This allows the user to control alcohol intake. Diabetic patients may be told by their doctor to have no-alcohol days. In this case, in the present embodiment, on no-alcohol day per week set by a user, any dish containing alcohol may not be displayed on a personalized menu.

Salt intake is restricted because high salt intake can result in an increase in the blood pressure, which may strain the kidney. For people without hypertension, it is recommended to limit the maximum daily intake of salt to 8 g/day for adult men and 7 g/day for adult women. For people with hypertension, more stringently, the maximum daily intake of salt may be limited to 5 g/day or less. In the present embodiment, the amount of salt contained in a dish may be displayed on a personalized menu, or only dishes whose salt content is reduced depending on the blood pressure of the patient are displayed on the personalized menu. Thus, the user can recognize the amount of salt contained in the meal and can manage the amount of salt intake.

Diabetic patients are subject to strict restrictions on the calorie intake and the blood sugar level, and thus there is a problem that the amount of food they are allowed to eat is reduced and it is difficult to obtain a feeling of fullness. One way of satisfying a desire of a diabetic patient to want to eat a hearty meal is to allow the diabetic patient to eat a dish including an ingredient containing a large amount of dietary fiber thereby giving a feeling of fullness. Furthermore, the intake of dietary fiber may have the effect of suppressing the rise in blood sugar level. In the present embodiment, a recommendation of a dish containing a large amount of dietary fiber may be displayed on a personalized menu. Alternatively, in the present embodiment, a dish may be combined with a dish containing a large amount of dietary fiber, and a resultant set of dishes may be displayed on a personalized menu.

Dietary constraints and recommendations are also applicable to diseases other than diabetes. An example of such a disease that needs dietary restrictions is hyperlipemia. For patients with hyperlipemia and with high cholesterol, it is generally recommended to limit calorie intake and limit cholesterol intake, and recommended to have high intake of polyunsaturated fat (rich in vegetable oil and fish oil), high intake of dietary fiber, etc. Examples of cholesterol-rich foods include liver such as chicken liver, pork liver, and beef liver, squid, sinew, and egg yolk. In the present embodiment, a dish whose contents of the ingredients described above are reduced or excluded may be displayed on a personalized menu for a user with hyperlipemia and with high cholesterol.

For patients with hyperlipidemia with high neutral fat levels, it is generally recommended to limit calorie intake, limit glucide intake, and limit alcohol. In the present embodiment, for a patient with hyperlipidemia having high neutral fat, dishes in which calories, glucide, and alcohol are reduced or excluded may be displayed on a personalized menu.

Gout is another example of a disease that requires dietary restrictions. For patients with gout, correction of obesity (reducing weight), restriction of calory intake, restriction of purine intake, restriction of alcohol intake, and high water intake are generally recommended. Examples of ingredients containing much purine include shrimp, dried squid, liver, and the like. In the present embodiment, for a patient with gout, dishes in which ingredients such as those described above are reduced or excluded may be displayed on a personalized menu.

Hypertension is another example of a disease that requires dietary restrictions. For patients with hypertension, restriction of salt intake, correction of obesity (weight loss), and restriction of alcohol are generally recommended. In the present embodiment, for a user with hypertension, a dish containing low salt, low alcohol, and low calories may be displayed on a personalized menu.

It should be noted that the dietary restrictions described above are merely examples, and different dietary restrictions may be imposed. Furthermore, the diseases described above are also merely examples, and the present embodiment can be applied in a similar manner to other diseases that require dietary restrictions.

In the present embodiment, dietary restrictions are not limited to those described above, but the dietary restrictions may be appropriately changed depending on the progress of the disease. For example, in FIG. 22, the maximum acceptable daily intake of alcohol for diabetics is 25 g/day or less. However, this is a general value, and no consideration is taken into account on the progress of the disease of the specific patient. As diabetes progresses, the maximum acceptable daily intake of alcohol is decreased, and eventually abstinence from alcohol may be required. In the present embodiment, when diabetes worsens, a personalized menu may be displayed such that only alcoholic beverages having an alcohol content less than the acceptable value can be selected, or alcohols may not be displayed on the personalized menu at all.

In hypertension, for example, the maximum acceptable salt intake may be 10 g/day or less for preventing hypertension, 7 g/day or less during hypertension treatment, and 5 g/day or less for patients in more severe status. As described above, for patients with hypertension, the maximum acceptable daily intake of salt may be reduced depending on the degree of hypertension. In the present embodiment, for a patient with hypertension, when the hypertension worsens, dishes may be displayed on a personalized menu such that the higher the salt content of the dish, the lower the priority in displaying. More specifically, for example, dishes containing a large amount of salt may be displayed in an inconspicuous manner, or in places that are difficult to select, or hidden in a personalized menu. Furthermore, in the present embodiment, when a dish is selected, the total salt content of the dish may be displayed, and/or another dish may be displayed which is recommended to be combined with the former dish.

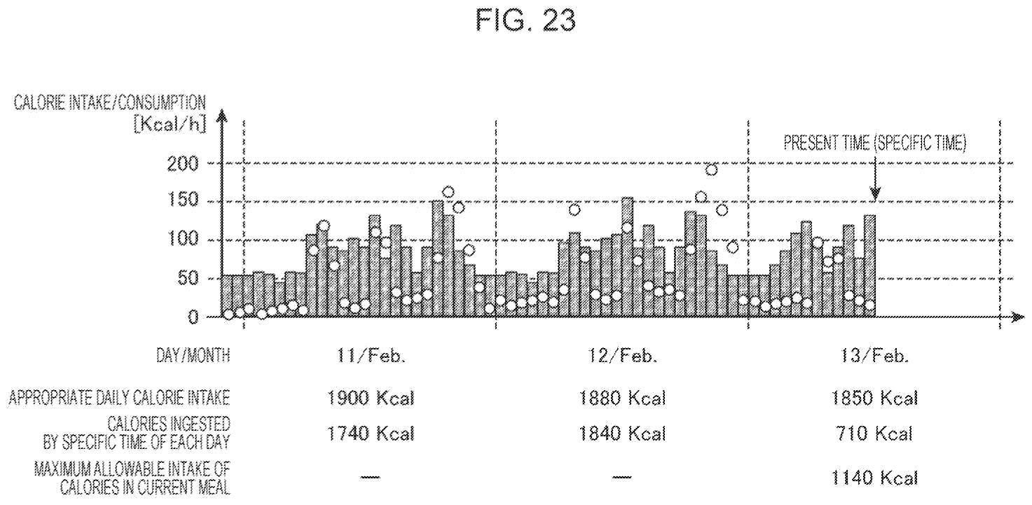

Next, taking calories as an example, methods of calculating the acceptable maximum acceptable intake in a current meal in the 7th to 17th variations described above are described below. FIG. 23 is a graph for explaining an example of calculating a maximum acceptable intake of calories in a current meal. In this graph, a vertical axis represents calorie intake and calorie consumption, and a horizontal axis represents time. In this graph, each vertical bar represents calories consumed by a user in one hour, and each open circle represents calories ingested in one hour. The calorie consumption may be measured using an acceleration sensor and/or heart rate sensor worn by a user, and while the calorie intake may be measured using a bioimpedance sensor and/or a blood sugar level sensor.

The appropriate daily calorie intake can be calculated using equation (1) described later with reference to FIG. 37. For example, the appropriate daily calorie intake on February 11 is calculated as 1900 Kcal, 1880 Kcal on February 12, and 1850 Kcal on February 13 (today). The “calories ingested by a specific time” indicates the calories ingested by a present time (a specific time) on a day. The time pointed to by a “present time” arrow in FIG. 23 indicates, for example, the time at which a personalized menu is generated. The calorie ingested by the specific time today is 710 Kcal. The maximum acceptable intake of calories in a current meal is calculated by subtracting the calories ingested by the present time from the appropriate daily calorie intake. Therefore, on February 13, the maximum acceptable intake of calories in the current meal is calculated as 1850 Kcal-710 Kcal=1140 Kcal.

In this case, the computational processing unit 104 of the information terminal 100 generates a personalized menu from the menu information such that a high displaying priority is given for dishes having calories equal to or smaller than 1140 Kcal.

In FIG. 23, calorie is taken as an example, but the target intake of an ingredient in a current meal and the maximum acceptable intake of an ingredient in the current meal can also be calculated in a similar manner as described above with reference to FIG. 23. More specifically, for example, the target intake of a specific ingredient in the current meal is calculated by subtracting the cumulative intake of the specific ingredient until the present time today from the target intake of the specific ingredient today. The computational processing unit 104 then generates a personalized menu from the menu information such that high displaying priority is given to dishes whose contents of the specific ingredient are equal to or greater than the target intake in the current meal.

The maximum acceptable intake of a specific ingredient in a current meal can be calculated by subtracting the cumulative intake of the specific ingredient until the present time today from the maximum acceptable intake of the specific ingredient today. The computational processing unit 104 then generates a personalized menu from the menu information such that high displaying priority is given to dishes whose contents of the specific ingredient are equal to or smaller than the maximum acceptable intake of the specific ingredient in the current meal. As the specific ingredient, for example, an ingredient containing a predetermined nutrient depending on the disease may be used. Alternatively, the specific ingredient may be an ingredient containing a nutrient whose content is greater than a recommended amount indicated by a vector (for example, an amount-of-salt vector) corresponding to a nutrient which is specified to be restricted by dietary restriction conditions indicated by equation (1) described later with reference to FIG. 37. Alternatively, the specific ingredient may be an ingredient containing a nutrient whose content is lower than a recommended amount indicated by a vector (for example, an amount-of-dietary-fiber vector) corresponding to a nutrient which is specified to be recommended.

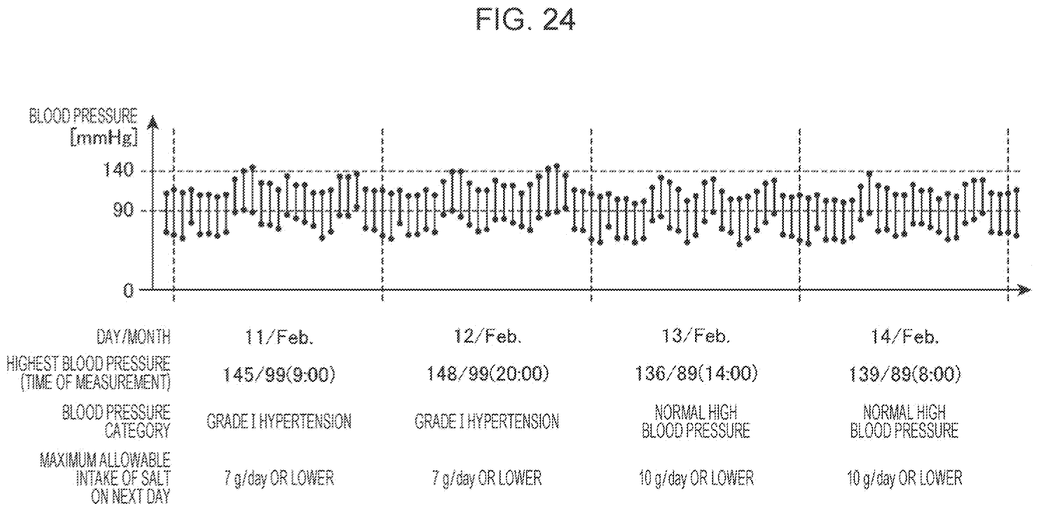

Next, a relationship between the blood pressure and the salt intake for a hypertensive patient is described below. FIG. 24 is a graph showing a relationship between the blood pressure and the salt intake for a hypertensive patient. In FIG. 24, a vertical axis represents the measured value of the blood pressure of a user, and a horizontal axis represents time.

The measured value of the blood pressure may be acquired using a blood pressure sensor built in a wristband worn by the user. This graph shows values of the blood pressure measured at intervals of one hour. The upper end of each bar indicates a systolic blood pressure measurement at a particular time, and the lower end of the bar indicates a diastolic blood pressure measurement at that particular time.

In this example, the highest blood pressure on February 11 was 145/99. In this specific case, the highest blood pressure falls into a class of mildest hypertension called “grade I hypertension”. The highest blood pressure on February 12 was 148/99, which is classified as “grade I hypertension” as on the previous day. The highest blood pressure on February 13 was 136/89. This highest blood pressure is within a range of “normal high blood pressure”, which is not hypertension. The highest blood pressure on February 14 was 139/89. This highest blood pressure is also within the range of “normal high blood pressure” as on the previous day.

In the “maximum acceptable intake of salt on next day” shown in the bottom row below the horizontal axis, the maximum acceptable intake of salt is creased on February 13 from 7 g/day or less to 10 g/day or less. This is because the blood pressure was in the range of the class of the grade I hypertension until February 12, but the blood pressure on February 13 was in the normal high blood pressure range, which indicates that the hypertension has improved, and this situation is maintained after February 13. In the present embodiment, it is possible to measure the value of a major marker of a disease in real time with the biosensor 600, and relax (or strengthen) the dietary restrictions according to the improvement (or deterioration). In this way, based on ever-changing biological information of the user, the contents of the meal to be ingested and the exercise to be performed by the user can be updated and proposed to the user via the information terminal 100 or the like.

For example, the calculation unit 104 generates a personalized menu as follows: The acceptable daily intake of salt is set to 7 g until February 13, because in this period, the blood pressure measured previous to each day was in “grade I hypertension”. However, on a day previous to February 14, the blood pressure fell in the class of “normal high blood pressure”, and thus the acceptable daily intake of salt is relaxed to 10 g on February 14 and on following days.

In the above-described example, only the highest blood pressure was checked. However, the acceptable daily intake of salt may be set also taking into consideration the diastolic blood pressure, or may be set taking into consideration the average or dispersion of blood pressure values. Alternatively, the acceptable daily intake of salt may be set based on the blood pressure measured not each day but measured at longer or shorter intervals. Alternatively, in order to exclude the blood pressure measured when a user is exercising, other information in terms, of for example, the heart rate or the activity amount may be taken into consideration in setting the acceptable amount of salt intake.

### Data Structure

Next, data structures of various kinds of data used in the present embodiment are described below. FIG. 25 is a diagram showing an example of a data structure of information 2500 including a diagnosis result of a periodic medical examination of a user managed by a first server. In this information 2500, fields and values are described in association with each other such that a computational process can be easily performed. One file of information 2500 is configured in a format such as JSON (JavaScript (registered trademark) Object Notation).

A field of “information category” is a field indicating what kind of personal information the information 2500 is. In this specific example, the information 2500 is about health and thus “health” is stored as the value in the information category field. The “information category” field is located at the beginning of the information 2300. Note that information whose “information category” is “health is treated as having a food attribute.

An “issuer” field is a field for identifying a legal entity that issued this information 2500. In this specific example, the diagnosis result of the periodic medical examination was generated by ABC clinic, and thus ABC clinic is described as the value in the “issuer” field.

A “date of issue” field is a field for indicating a date and time of issue made by the issuer of this information 2500. In this example, Feb. 15, 2020 is described as the value in the “date of issue” field. The value in this field may include a time of issue in addition to the date of issue. The value in this field may further include time zone information.

A “data type” field is a field for specifically identifying a content of this information 2500. In this specific example, “medical examination information” is described by way of example as the value in the “data type” field. This indicates that data described in following fields are data related to a result of diagnosis of a periodic medical examination on the user.

The information terminal 100 may interpret that the information 2500 includes information indicating a result of diagnosis result of a periodic medical examination based on values described in the respective fields of “information category” and “data type”. Alternatively, the information terminal 100 may interpret that the information 2500 includes information indicating a result of diagnosis result of a periodic medical examination based on information related to the information 2500 (for example, the file name of the information 2500).

A “date of measurement” field is a field indicating the date and time of executing the periodic medical examination indicated by the value of “medical examination information” described in the preceding field of “data type”. In this example, Feb. 14, 2020 is described as the value in the “date of measurement” field. This value may include time in addition to the measurement date. The value in this field may further include time zone information. In a case where a plurality of diagnosis results of periodic medical examinations for a user corresponding to the first server 200 are stored, information 2500 with the latest date described in the “date of measurement” field may be used.

The fields and values following the “date of measurement” field describe results of diagnosis on the user in terms of the height, the weight, the systolic blood pressure, the diastolic blood pressure, the red blood cell count, the hemoglobin count, the HDL cholesterol level, the LDL cholesterol level, the fasting blood sugar level, and the uric acid level.

FIGS. 26A and 26B are diagrams each showing an example of a data structure of the information 2600 including contents of a medical certificate on a user managed by the first server 200. The medical certificate is issued by a medical institution to which the user goes regularly. The medical certificate includes information on a disease of the user. Information 2601 shown in FIG. 26A includes contents of the medical certificate in terms of diabetes, and information 2602 shown in FIG. 26B includes contents of the medical certificate in terms of hypertension.

The information category, publisher, issue date, and date of measurement are the same as those of the information 2500. As the value corresponding to the “data type” field, “medical certificate” is described to indicate that the information 2600 is of the medical certificate.

The name of the disease of the user is described as the value of the “disease name” field. In a case where the value of the “disease name” field is “diabetes”, the following fields and values contain data indicating the degree of the disease related to the diabetes. More specifically, information 2601 includes descriptions of “disease onset date”, “disease type”, “HbA1c (NGSP) [%]”, and “fasting blood sugar level”.

“HbA1c (NGSP) [%]” is an international standard measure that indicates the proportion of glycosylated hemoglobin, and the value thereof depends on the average blood sugar level for the past two months. For example, normal values are less than 5.6, caution values are in a range from 5.6 to 5.9, reserve values are in a range from 6.0 to 6.4, and positive values are 6.5 or more. In this specific example, “6.9” is described as the value of “HbA1c (NGSP) [%]” for this user, and this measured value is within a range in which diabetes is positive.

The fasting blood sugar level [mg/dl] is the minimum blood sugar level measured after fasting over 10 hours or more. For example, normal values are in a range from 80 to 99. The fasting blood sugar level from 100 to 125 is considered prediabetes, and the level 126 or higher indicates diabetes. In this specific example, 131 is described as the value of the fasting blood sugar level of the user, and this measured value is within a range in which diabetes is positive.

In a case where “hypertension” is described as the value of the “disease name” field, the following fields and values contain data indicating the degree of the disease of hypertension.

More specifically, information 2602 includes descriptions of “disease onset date”, “category”, “systolic blood pressure [mmHg]”, and “diastolic blood pressure [mmHg]”.

The “category” indicates a category of hypertension according to the blood pressure value. There are three categories of blood pressure in descending order: optimal blood pressure, normal blood pressure, and normal high blood pressure. These are classified as normotensive. In addition, there are four categories: grade I hypertension; grade II hypertension; grade III hypertension; and systolic hypertension. These are classified as hypertension. In this specific example, “grade I hypertension” is described as the value of the category, which indicates that the user is classified as mild hypertension.

As values of the “systolic blood pressure [mmHg]” field and the “diastolic blood pressure [mmHg]” field, blood pressure values are described. In this specific example, 155 and 92 are described as the values, which indicates that the blood pressure of the user is classified in the grade I hypertension.

The information 2600 including diagnosis contents of a medical certificate issued by a medical institution (ABC clinic in this specific example) such as those described above and further including a measurement date and a date of issue is stored in the first server 200. Therefore, it is possible to correctly grasp the past medical history of the user and the change in the disease status based on the date described in the value of the “date of measurement” field of the medical certificates. Furthermore, based on the date described in the value of the “date of measurement” it is also possible to refer to the latest disease diagnosis results.

In this specific example, there is no field for identifying the user, but the user can be identified by user identification information included in, for example, a file name.

FIG. 27 is a diagram showing an example of a data structure of information 2700 including biological information managed by the first server 200.

The information 2700 includes measurement date/time, a systolic blood pressure, a diastolic blood pressure, a calorie intake, a calorie consumption, a salt intake, an alcohol intake, HbA1c, and a blood sugar level, etc. The measurement date/time include a year, month, day, hour, minute, and second, expressed in Japan standard time. As for the systolic blood pressure, the diastolic blood pressure, HbA1c, and the blood sugar level, values thereof measured at the date/time shown in the measurement date/time are described. As for the calorie intake, the calorie consumption, and the alcohol intake, values are measured at the date/time shown in the measurement date/time, and converted to values per hour and the resultant values are described. As for the salt intake, the value indicating the intake on the day previous to the data described in measurement date is calculated and described.

These measurement values are directly or indirectly derived from values measured by or more biosensors 600.

For example, the systolic blood pressure and the diastolic blood pressure may be measured with a blood pressure sensor. The calorie intake may be measured by a bioimpedance sensor and/or a blood sugar level sensor. The calorie consumption may be measured with an acceleration sensor and/or a heart rate sensor. The salt intake may be measured with a urine salt concentration sensor. The alcohol intake may be measured with a biogas sensor. The HbA1c may be measured with a near infrared laser sensor and/or a mid-infrared laser sensor. The blood sugar level may be measured with a blood sugar level sensor and/or a subcutaneous tissue glucose sensor.

When values of the same type of biological information are measured by two or more biosensors 600, the weighted average value of the measured values may be stored in the information 2700, wherein the weights may be given according to the sensor accuracy.

In the present example, the values measured by the biosensor 600 are stored in time series, but in the present disclosure there is no particular restriction on the manner of storing the measured values. For example, in a case where it is possible to acquire meal order history information of a user, the measured values described in the information 2700 may be complemented or corrected using the amounts of calorie, salt, alcohol, dietary fiber, cholesterol, etc., included in ordered dishes indicated by the order history information.

For example, in a case where the amount of alcohol is estimated by measuring a concentration distribution of volatile gas released from a skin using a biogas sensor, the estimated amount of alcohol may be low in accuracy. In this case, if the amount of alcohol beverage and the alcohol content thereof can be acquired from the order history information, the amount of alcohol may be calculated according to an equation shown below.

Amount of alcohol [g]=amount of alcohol beverage [ml]*alcohol content [%]/100*0.8 Similarly, if the amount of dietary fiber can be acquired from the order history information, the measurement data may be supplemented or corrected by using the amount acquired from the order history information. In this specific example, the information 2700 includes no field for identifying the user, but the user can be identified by user identification information included in, for example, a file name.

FIGS. 28A, 28B, and 28C are each a diagram showing an example of a data structure of ingredient information 2800. In the ingredient information 2800, fields and values are described in association with each other such that a computational process can be easily performed. The ingredient information 2800 is embedded together with a tag indicating the ingredient information in the menu information described in, for example, an HTML file format, which is acquired from the second server 300 of each restaurant company. However, this is merely an example, and the ingredient information 2800 may be given by a separate file described in a JSON format and prepared for each dish.

A “dish name” field is a field indicating a dish about which the present information describes. In an example of ingredient information 2801 shown in FIG. 28A, ramen set B is described as the value of the “dish name” field. In an example of ingredient information 2802 shown in FIG. 28B, ramen in soy sauce-based soup is described as the value of the “dish name” field. In an example of ingredient information 2803 shown in FIG. 28C, xiaolongbao is described as the value of the “dish name” field.

In fields and values following the “dish name”, a list of ingredients used is described. For example, the ingredient information 2801 is information about a cup of ramen in soy sauce-based soup and three pieces of xiaolongbao and thus the ingredient information 2801 includes a “ramen in soy sauce-based soup” field and a “xiaolongbao” field, “1” is described as the value of the ramen in soy sauce-based soup, and “3” is described as the value of the xiaolongbao. Since the ingredient information 2801 is ingredient information 2800 of a set of dishes, there are no fields and values related to an ingredient of a dish.

Since the ingredient information 2802 is ingredient information 2800 regarding the ramen in soy sauce-based soup as a single dish, ingredients and amounts thereof are described in an associated manner such that “flour”, “50 g”; “salt”, 5 g″; and so on. Similarly, in the ingredient information 2803 which is ingredient information 2800 regarding the xiaolongbao as a single dish, ingredients of the xiaolongbao and amounts thereof are described in an associated manner as with the ingredient information 2802.

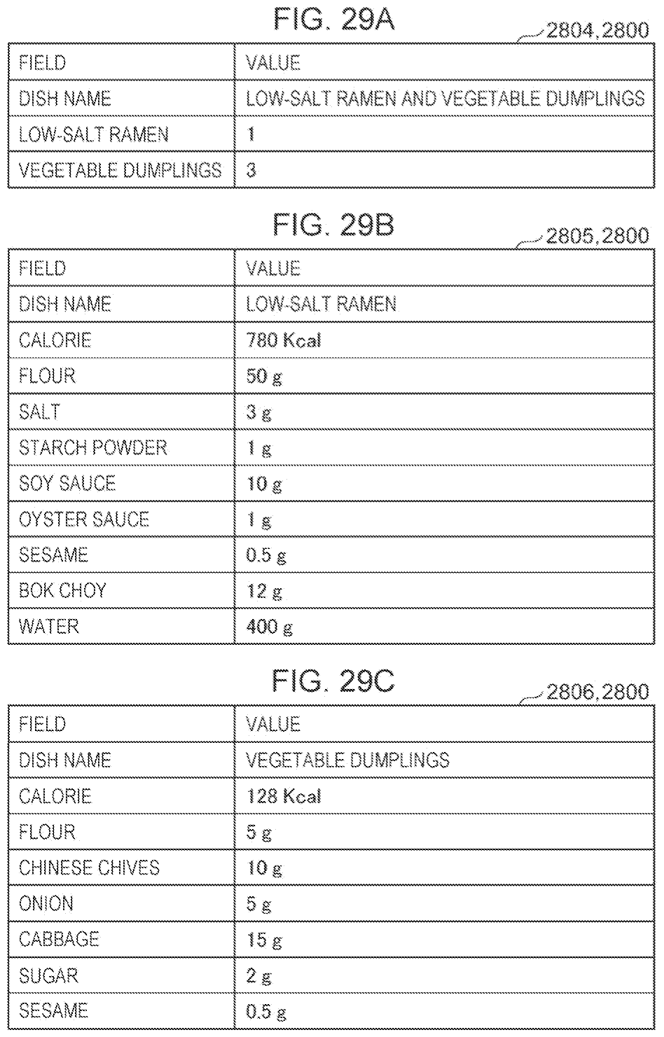

As shown in the ingredient information 2802, the ramen set B includes salt of 8 g, which is too much for diabetic patients. Furthermore, as shown in the ingredient information 2803, the xiaolongbao does not include a large amount of vegetables, and thus the amount of dietary fiber is not enough. Therefore, it is not appropriate for diabetic users to eat ramen set B as it is. Thus, the computational processing unit 104 of the information terminal 100 proposes, via a personalized menu, for example, a set of dishes including low-salt ramen and vegetable dumplings shown in FIGS. 29A to 29C to the user instead of the ramen set B.

FIGS. 29A, 29B, and 29C are diagrams showing examples of ingredient information 2804 of low-salt ramen and vegetable dumplings. In the low-salt ramen and vegetable dumplings, as shown in ingredient information 2805 in FIG. 29B and ingredient information 2806 in FIG. 29C, salt is reduced from 8 g to 3 g, bok choy is used instead of pork char siu, Chinese chives are used instead of ground pork, and cabbage and sugar are used instead of honey and chicken broth. Thus, “low-salt ramen and vegetable dumplings” allowed to be eaten by a diabetic user is displayed on the personalized menu. Thus, the user can smoothly order a dish from those allowed to be eaten by the user.

When a dish is replaced with another dish, the information terminal 100 checks the ingredient information 2802 shown in FIG. 28B and the ingredient information 2803 shown in FIG. 28C with respect to intake-restricted ingredients (such as alcohol, salt, etc.) specified by the information indicating the degree of dietary restriction, and extracts replacement candidate ingredients from the ingredient information 2802 and the ingredient information 2803.

Next, the information terminal 100 calculates the maximum acceptable intake of the replacement candidate ingredients in a current meal based on the information indicating the degree of dietary restriction. If the amount of a replacement candidate ingredient is equal to or greater than the maximum acceptable intake in the current meal, the information terminal 100 reduces the amount of this replacement candidate ingredient to a value smaller than the maximum acceptable intake in the current meal. When the amount of a replacement candidate ingredient is equal to or greater than the maximum acceptable intake in the current meal, if an alternative ingredient and an amount of the alternative ingredient are predetermined, the information terminal 100 rewrites the ingredient information 2802 and the ingredient information 2803 by substituting the predetermined alternative ingredient and amount of the alternative ingredient for the replacement candidate ingredient and the amount thereof. Furthermore, the information terminal 100 also replaces the dish name in the ingredient information 2800 with a predetermined dish name according to the replaced ingredient. As a result, ingredient information shown in FIGS. 29A to 20C is obtained. The information terminal 100 may replace dishes by performing the process described above for each of the dishes included in the acquired menu information.

In the above-described example, the replacement of ingredients of dishes are performed on the information terminal 100 based on the information indicating the degree of dietary restriction of the user, but the present disclosure is not limited to this example. For example, ingredient information (2802, 2803, 2805, 2806) for a single dish may be received from the second server 300, and the information indicating the degree of the dietary restriction of the user may be received from the first server 200, and then the information terminal 100 may propose a dish according to the information indicating the degree of dietary restriction of the user as a single dish or a set of dishes.

### Overall View of Process

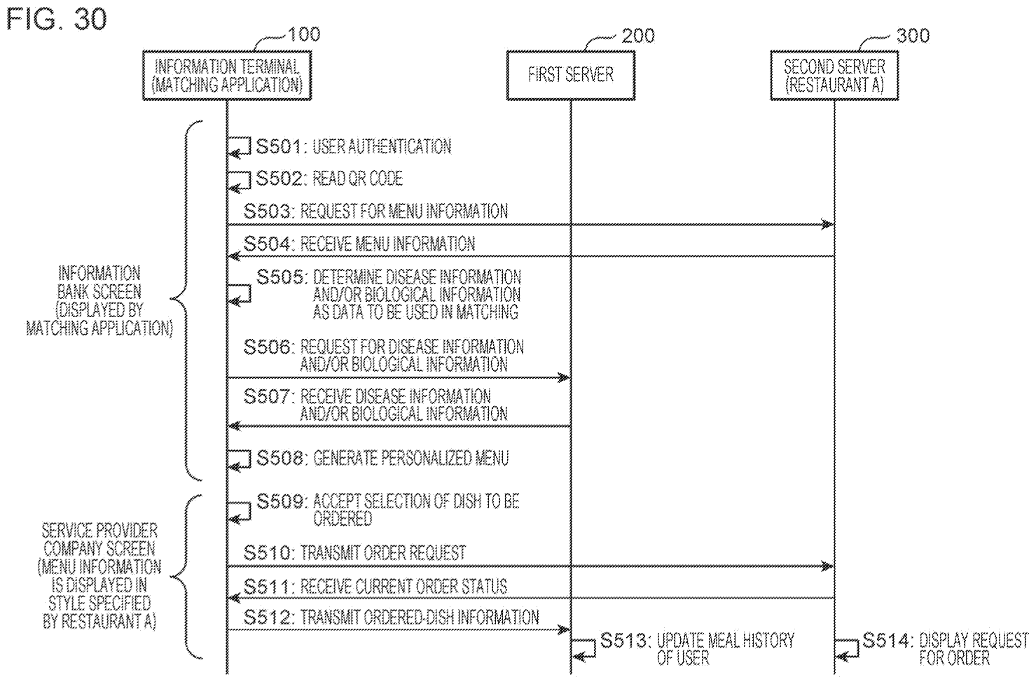

Next, an overall view of the process performed in the information processing system according to the present embodiment is described below. FIG. 30 is a sequence diagram showing an example of an overall view of the process performed in the information processing system according to the present embodiment.

After a user enters a store of the restaurant company A and is seated in a seat, if the user starts the matching application by operating the information terminal 100, the matching application performs user authentication (step S501). If the user authentication is passed successfully, the matching application displays the home screen G103 (see FIG. 14).

When the user touches the scan button 1402 on the home screen G103, the matching application activates the scan function and acquires a QR code corresponding to the seat of the user (step S502). As a result, the matching application acquires the URL of the second server 300 (HTTP server) of the restaurant company A to which the connection is to be made.

After the matching application acquires the URL, the matching application transmits a request for acquiring the menu information (for example, an HTTP request) to the second server 300 of the restaurant company A based on the URL (step S503). In this step, as described above, the matching application may transmit the seat ID included in the QR code to the second server 300.

In response to receiving this request, the second server 300 transmits an HTTP response thereby returning the menu information by using the HTTP server function. The matching application receives the menu information of the restaurant company A (step S504).

When the matching application receives the menu information, the matching application analyzes the received menu information. In this case, it is detected as a result of the analysis that the data attribute of the received menu information is the food attribute. More specifically, for example, the matching application may detect the food attribute by analyzing the contents of the menu information. Alternatively, the matching application may detect the food attribute from supplementary information transmitted separately from the menu information. When the matching application detects that the data attribute of the received menu information is the food attribute, the matching application determines that the disease information and/or biological information is to be used as the data for matching (step S505).

Next, to request for providing the disease information and/or biological information of the user who has passed the authentication successfully, the matching application transmits an HTTP request to the first server 200 based on the user ID of the user (step S506). When the first server 200 receives this HTTP request using the function of the HTTP server, the first server 200 extracts the disease information and/or biological information of the user from the memory 203 based on the user ID, and transmits an HTTP response to the matching application by returning the extracted disease information and/or biological information. Thus, the matching application receives this disease information and/or the biological information related to the user (step S507).

When the matching application receives the disease information and/or the biological information, the matching application calculates the information indicating the degree of dietary restriction based on the received disease information and/or biological information, and generates a personalized menu using one of the 17 variations described above based on the calculated information indicating the degree of dietary restriction and the received menu information such that the personalized menu meets the information indicating the degree of dietary restriction of the user (step S508).

The matching application generates various screen style UI (User Interface) designs displayed in the process from step S501 to step S508 according to the styles of the matching application. However, the styles (for example, UI designs) specified by the restaurant company A are employed when the matching application generates various screens staring from the screen for the personalized menu ending to the screen for use in the dish order. In other words, although each service providing company (for example, each restaurant) uses the matching application provided by another company (for example, an information bank or an information intermediary), the service providing company can communicate with users (for example, customers) in a specific style (for example, a UI design) the service providing company wants to use. This means that the above-described standard menu and the personalized menu can both be expressed in the style (for example, a UI design) specified by the restaurant company A while maintaining consistency. Regarding the styles of various screens (for example, UI designs) used for the personalized menu and in various processes until the order is completed, screens may be generated by laying out materials (characters explaining dishes, pictures of dishes, etc.) prepared by the restaurant company A according to a style specified by an administrator of the first server 200 (an information bank). This makes it possible to provide a unified user experience to users who use the matching application.

After the matching application generates the personalized menu, the matching application displays the operation screen G106 including the generated personalized menu in the style specified by the restaurant company A, and accepts a selection instruction which is issued by the user to order a dish from the personalized menu (step S509).

When the matching application receives the selection instruction, the matching application transmits an order request including the seat ID of the user and the ordered-dish information indicating the dish to be ordered in association with each other to the second server 300 of the restaurant company A (step S510). In a case where the ingredient information 2800 of the ordered dish has been changed from the menu information received from the second server 300, the matching application may transmit the ordered-dish information together with the changed ingredient information 2800 to the second server 300. This makes it possible for the store of the restaurant company A to provide the user with the dish in which the substitute ingredient and the amount of the substitute ingredient are reflected. In a case where the ingredient information 2800 is different from the information received in step S504, the matching application may ask the second server 300 whether or not the change in the ingredients can be handled before the matching application generates the personalized menu in step S508.

In response to receiving the order request, the second server 300 returns to the matching application a response confirmation (ACK) indicating that the order has been received and also, as necessary, the current order status (for example, information about the order history screen G108). Thus, the matching application receives the current order status (step S511).

The matching application also transmits the ordered-dish information in association with the user ID to the first server 200 (step S512) and requests the first server 200 to add it to the meal history information of the user or update the meal history information of the user. In response to receiving the ordered-dish information, the first server 200 updates the meal history information related to the user according to the received ordered-dish information (step S513). In this update step, a time stamp indicating the time of ordering the dish indicated by the ordered-dish information is also added to the meal history information.

The ordered-dish information transmitted in step S512 may include information indicating a dish name of a dish selected via the personalized menu, information indicating a price of the dish selected in via the personalized menu, information indicating a date/time when the dish selected via the personalized menu is ordered, information indicating a nutrient included in the dish selected via the personalized, and information indicating a restaurant company (and its store) to which the order is issued.

Upon receiving the order request, the second server 300 of the restaurant company A displays the order request on the display in the store (step S514). This allows an employee of the store to correctly serve the ordered dish to the user in the seat who ordered the dish.

In this way, the meal history information, which is part of the user's personal information, is accumulated in the first server 200 in a fine, accurate, and time-serial manner. This makes it possible to use this big data when a dish is ordered for the next time, which allows it present highly suitable dish options to the user with higher accuracy.

According to the control method shown in FIG. 30, it is possible to reduce the risk that a user with a dietary restriction is erroneously served a dish containing an ingredient that the user should avoid. Furthermore, according to the control method shown in FIG. 30, even for a user for whom it is necessary to make a confirmation of details of ingredients to be avoided, cooking method, etc., the user is allowed to easily order a dish without having a restaurant perform such confirmation, and the restaurant can easily handle the dish order from such a user. Furthermore, according to the control method shown in FIG. 30, it is possible to reduce the risk that a restaurant staff gets to know personal information related to the user's privacy such as disease information and/or biological information, and the risk that the user's personal information related to the privacy is accumulated in the store terminal.

Furthermore, according to the control method shown in FIG. 30, it is possible to effectively and safely manage personal information including accurate and temporally continuous disease information and/or biological information and meal history information (for example, order history information). Furthermore, according to the control method shown in FIG. 30, it is possible to prevent the personal information from being leaked to anyone other than the company to which permission is given by the user. Furthermore, according to the control method shown in FIG. 30, the user can safely place an order without paying attention to or considering the details and history of the disease information and/or biological information of the user.

### Flowchart of Order Process

Next, a process performed by the information terminal 100 according to the present embodiment is described below. FIG. 31 is a flowchart showing an example of a process which is performed by the information terminal 100 when a dish is ordered from the standard menu. The process shown in this flowchart is started when the user activates the QR code reader on the information terminal 100.

In step S11, the QR code reader reads a QR code provided on a seat of the user and transfers a read text string (for example, a URL) to the browser. In this process, the QR code is read via the operation screen G1 shown in FIG. 7, and the reading result of the QR code such as that shown in FIG. 8 is displayed on the operation screen G2.

In step S12, the browser accesses the second server 300 of the restaurant company A according to the URL, acquires the menu information, and displays the standard menu on the display 105 of the information terminal 100. In this process, the operation screen G3 including the standard menu such as that shown in FIG. 9 is displayed.

In step S13, the browser accepts an instruction from the user to select a dish to order from the standard menu. In this step, the user operates the standard menu included in the operation screen G3 shown in FIG. 10 to select the dish to be ordered.

In step S14, the browser transmits an order request including the user's seat ID and the ordered-dish information indicating the dish ordered by the user in association with each other to the second server 300 of the restaurant company A. Thus, the order process via the standard menu is completed.

As described above, when a user wants to order an appropriate dish prepared taking into consideration the information indicating the degree of dietary restriction of the user at a restaurant, the user may display a personalized menu using a matching application and may order a dish via the personalized menu. On the other hand, when a user wants to select a dish from a standard menu of a restaurant without considering the information indicating the degree of dietary restriction of the user, the user may display the standard menu using a general-purpose QR code reader and may order a dish from the standard menu.

In each case, a seat ID read from a QR code corresponding to a seat in which a user is seated is sent together with the ordered-dish information to the second server 300, and thus the ordered dish is correctly served.

FIG. 32 is a flowchart showing an example of a process which is performed by the information terminal 100 when a dish is selected from a personalized menu and ordered. The process shown in this flowchart is started when a user activates the matching application to order a dish at a restaurant.

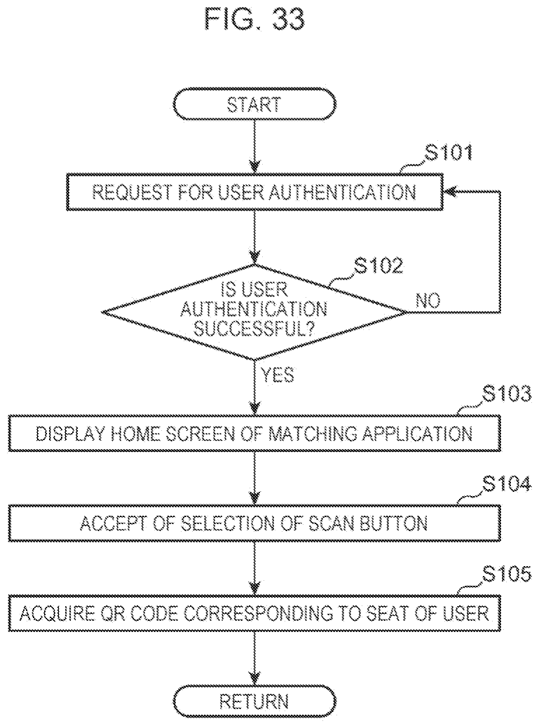

In step S1, the matching application executes a process of acquiring a QR code corresponding to a seat of the user. Details of this process are described later with reference to FIG. 33. In this process, the QR code is read via the operation screen G104 shown in FIG. 15, and the seat ID and restaurant ID are acquired. This process corresponds to steps S501 and S502 in FIG. 30.

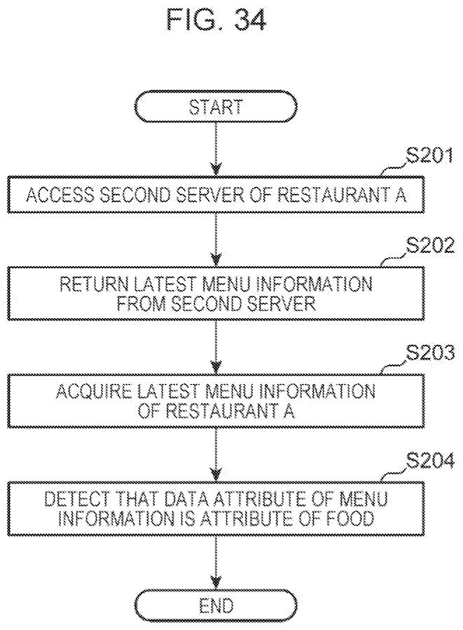

In step S2, the matching application accesses a URL indicated by the acquired QR code by using a browser function, and executes a process of acquiring menu information of the restaurant company A from the second server 300 of the restaurant company A. Details of this process will be described later with reference to FIG. 34. In this process, it is also detected that the data attribute of the menu information is the food attribute. In this process, the display screen G105 shown in FIG. 17 is displayed on the information terminal 100. This process corresponds to steps S503 and S504 in FIG. 30.

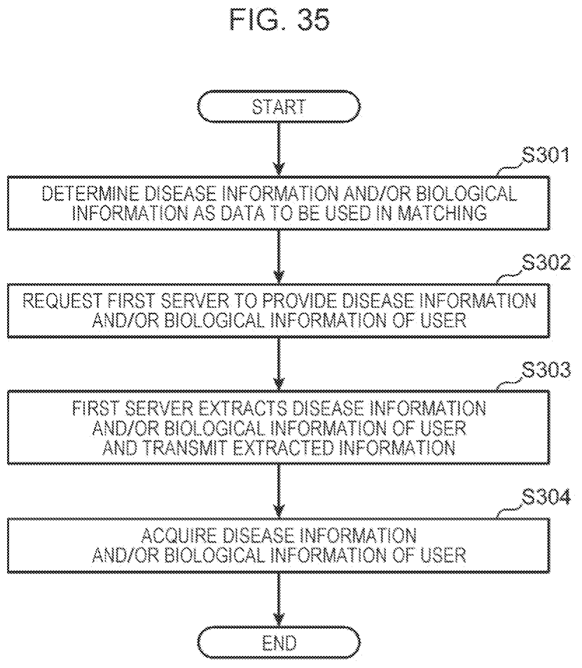

In step S3, the matching application executes a process of acquiring disease information and/or biological information, which is personal information related to the food attribute, included in the user's personal information from the first server 200. Details of this process will be described later with reference to FIG. 35. Via this process, the disease information and/or biological information is acquired which is related to the user seated in the seat indicated by the seat ID acquired in step S1. This process corresponds to steps S505, S506, S507 in FIG. 30.

In step S4, the matching application checks the menu information of the restaurant company A with respect to the information indicating the degree of dietary restrictions of the user, and generates a personalized menu including a dish so as to meet the degree of dietary restrictions. As a result of this process, the operation screen G106 including the personalized menu is displayed. This process corresponds to step S508 in FIG. 30.

In step S5, the matching application accepts an instruction issued by the user to select a dish to be ordered from the personalized menu. More specifically, for example, a dish is selected via the operation screen G106 shown in FIG. 19. This process corresponds to step S509 in FIG. 30.

In step S6, the matching application transmits, to the second server 300 of the restaurant company A, an order request including the ordered-dish information indicating the dish selected in step S5 and, in association with it, the seat ID acquired in step S1. This process corresponds to step S510 in FIG. 30.

The process of ordering a dish from the personalized menu is described in further detail below. FIG. 33 is a flowchart showing the details of the process in step S1 in FIG. 32. In step S101, when the matching application is activated by the user, the matching application prompts the user to perform an authenticate process. More specifically, for example, the user authentication process is performed via the authentication screen G101 shown in FIG. 12 or the authentication screen G102 shown in FIG. 13.

In step S102, the matching application determines whether or not the user has been successfully authenticated. In a case where the user authentication fails (NO in step S102), the process returns to step S101. When the user authentication is successful (YES in step S102), the process proceeds to step S103. In step S103, the matching application displays the home screen G103 of the matching application as shown in FIG. 14.

In step S104, when the matching application accepts a user operation of touching the scan button 1402 on the home screen G103, the matching application activates the QR code reading function. As a result, the operation screen G104 shown in FIG. 15 is displayed. In the mode in which a restaurant ID and a seat ID are read using NFC, an operation screen G1011 shown in FIG. 16 is displayed.

In step S105, the orientation and the position of the information terminal 100 are adjusted by the user, and the matching application reads the QR code corresponding to the seat in which the user is seated.

FIG. 34 is a flowchart showing the details of the process in step S2 in FIG. 32. In step S201, the matching application accesses the second server 300 of the restaurant company A based on a character string (for example, a URL) described in the QR code. This access is performed, for example, by an HTTP request.

In step S202, the second server 300 of the restaurant company A returns the latest menu information to the matching application by using the HTTP server function. This response is given, for example, by an HTTP response.

In step S203, the matching application acquires the latest menu information of the restaurant company A.

In step S204, the matching application detects, from the acquired menu information, that the data attribute of the menu information is the food attribute. This detection may be performed, for example, based on the data type described in an HTML file of the menu information, or based on data attribute identification information (an HTML tag).

FIG. 35 is a flowchart showing the details of the process in step S3 in FIG. 32. In step S301, since the data attribute of the acquired menu information is the food attribute, the matching application determines the disease information and/or biological information of the user as data to be used in matching.

In step S302, the matching application requests the first server 200 to provide the disease information and/or biological information of the user to be subjected to the matching. More specifically, the matching application specifies the user ID and requests that the disease information and/or biological information related to the specified user ID is returned in a predetermined encrypted form.

In step S303, the first server 200 extracts the disease information and/or biological information based on the user ID from a huge amount of personal information managed in a distributed and encrypted manner, shapes this disease information and/or biological information into a predetermined format, encrypts it in a predetermined manner, and returns it to the matching application.

In step S304, the matching application decodes the acquired latest disease information and/or biological information. As a result, the user's disease information and/or biological information is acquired.

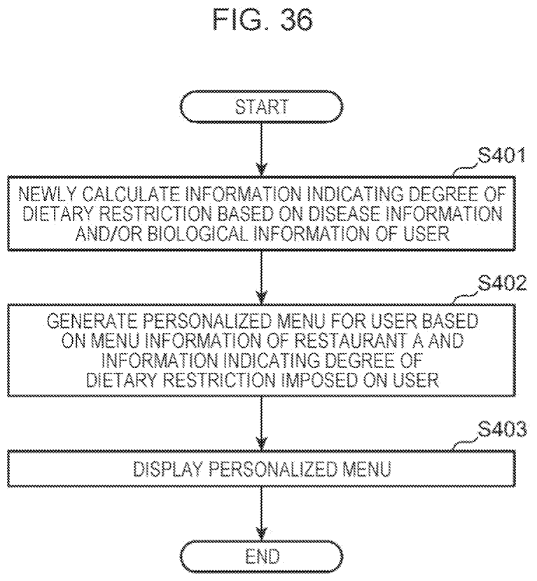

FIG. 36 is a flowchart showing the details of the process in step S4 in FIG. 32. In step S401, the matching application newly calculates information indicating the degree of dietary restriction imposed on the user based on the user's disease information and/or biological information. Details of this process will be described later with reference to FIG. 37.

In step S402, the matching application checks the menu information of the restaurant company A with reference to the information indicating the degree of dietary restriction, and generates a personalized menu so as to meet the dietary restriction imposed on the user. The menu information includes, as described above, one or more ingredients and the amount thereof included in each dish. The menu information may include cooking method information indicating a cooking method of each dish. In this process, the personalized menu is generated using one of the 17 variations described above.

In step S403, the matching application generates the operation screen G106 including the generated personalized menu and displays it on the display 105 of the information terminal 100 using the browser function. More specifically, for example, the personalized menu is displayed in a style specified by the display style information included in the menu information of the restaurant company A. Information indicating the degree of dietary restrictions

Next, a process of calculating information indicating the degree of dietary restriction is described below. FIG. 37 is a diagram showing equation (1) for calculating information indicating the degree of dietary restriction.

In equation (1), a matrix named dietary restriction condition (hereinafter, referred to as the dietary restriction condition matrix) corresponds to the information indicating the degree of dietary restriction. The dietary restriction condition matrix includes an amount-of-calorie vector, an amount-of-alcohol vector, an amount-of-salt vector, and an amount-of-dietary-fiber vector. These vectors are each an N×1 matrix.

The amount-of-calorie vector and the amount-of-salt vector each includes, as components, a prohibited amount, a caution amount, a recommended amount, and a required amount. The prohibited amount indicates an amount above which intake is extremely likely to cause the disease to become worse. The caution amount indicates an amount, smaller than the prohibited amount, above which intake is likely to cause the disease to become worse. The prohibited amount is an example of the maximum acceptable intake in the predetermined period. The recommended amount indicates an amount which is smaller than the caution amount and which is an optimum amount, that is, neither excessive nor deficient. The required amount is an amount which smaller than the recommended amount and which is a minimum amount required to sustain life. The amount-of-alcohol vector includes, as components, a prohibited amount, a caution amount, and a permitted amount. The permitted amount indicates an amount, smaller than the prohibited amount, and when intake is smaller than this permitted amount, it is likely that the disease will improve or will not worsen further. The amount-of-dietary-fiber vector includes, as components, a recommended amount and a required amount. The recommended amount indicates an optimum amount which is neither excessive nor deficient, and when intake is more than this recommended amount, it is likely that the disease improves. The recommended amount is an example of the target intake in the predetermined period described above. The required amount indicates a minimum amount that must be taken, and if intake is smaller than this required amount, it is likely that the disease will not improve or the disease will worsen.

A matrix C is a transformation matrix for obtaining a dietary restriction condition from a matrix represented by disease information+biological information. The matric C is calculated statistically or for each user in advance.

The matrix represented by disease information+biological information includes a medical examination vector, a systolic blood pressure vector, a diastolic blood pressure vector, a blood sugar level vector, a calorie intake vector, and a calorie consumption vector. A medical examination vector includes, as component, a date of measurement, a height, a weight, a systolic blood pressure, a diastolic blood pressure, etc. included in the information 2500 (the diagnosis result of the periodic medical examination). The medical examination vector may include various data included in the information 2600 (the diagnosis contents of the medical certificate).

The systolic blood pressure vector, the diastolic blood pressure vector, the blood sugar level vector, the calorie intake vector, and the calorie consumption vector respectively include measured values of the systolic blood pressure, the diastolic blood pressure, the blood sugar level, the calorie intake, and the calorie consumption which are measured at intervals of 1 hour in a period from 72 hours before to the latest time and stored as biological information in the information 2700. In this specific example, the values measured in the last three days and stored in the information 2700 are used, but this is merely by way of example. Values measured in a longer period (4 days, 1 week, 1 month, etc.) may be used, or contrarily, values measured in a shorter period (1 day, 2 days, etc.) may be used. Note that the biological information used in the matrix of disease information+biological information may be given by biological information other than the biological information shown by way of example in FIG. 37.

The matrix C can be calculated, for example, as follows. In the memory 203 of the first server 200, a huge amount of meal history information has been accumulated wherein, in the meal history information, the systolic blood pressure, the diastolic blood pressure, the blood sugar level, the calorie intake, and the calorie consumption measured when each user ate a meal are stored, and in association with them, the contents of the influence of the meal on the user, and the physical information of the user are also stored. The contents of the influence include changes in the calorie intake, the alcohol intake, the salt intake, and the dietary fiber intake. The physical information includes the height, the weight, the systolic blood pressure, the diastolic blood pressure, etc. obtained by the latest periodic medical examination.

As for at least one of the calorie intake, the alcohol intake, the salt intake, and the dietary fiber intake, a criterion in terms of a change in value required for improving the disease of the user is determined in advance for each user. From each piece of the huge amount of meal history information in terms of the calorie intake, the alcohol intake, the salt intake, and the dietary fiber intake, the first server 200 calculates changes in the amount-of-calorie vector, the amount-of-alcohol vector, the amount-of-salt vector, the amount-of-dietary-fiber vector required for satisfying the criterion. The first server 200 then calculates the matrix C by learning the relationship of the calculated amount-of-calorie vector, the amount-of-alcohol vector, the amount-of-salt vector, and the amount-of-dietary-fiber vector, with the systolic blood pressure, the diastolic blood pressure, the blood sugar level, and the calorie intake, and the calorie consumption.

Alternatively, the matrix C may be calculated such that a large amount of correlation data is stored in the memory 203 of the first server 200 in terms of how the matrix of the disease information+biological information of the user or an unspecified number of users changes before and after each meal including known ingredients, and a calculation is performed to determine the correlation between the meal ingested and the change in the biological information for the user or the unspecified number of users.

A further explanation is given below, in which, for the sake of simplicity, only the blood sugar level and the amount-of-calorie vector of the matrix C are considered. Let it be assumed here that it is predicted from analysis on the large number of meals and the biological information for the specific user or the unspecified number of users that if a user continues to eat meals with calories of X1, X2, and X3 [Kcal], respectively, probabilities causing the blood sugar level to increase gradually in a long term are 10%, 50%, and 80%, respectively. In this case, the recommended amount, the caution amount, and the prohibited amount of the amount-of-calorie vector are respectively given by X1, X2, and X3 [Kcal].

Note that the matrix C may be calculated by the information terminal 100 instead of the first server 200. In this case, when the first server 200 acquires a notification of calculation of the matrix C from the information terminal 100, the first server 200 may transmit the data necessary for the calculation of the matrix C to the information terminal 100.

In the above-described example, vectors of the dietary restriction condition and vectors of the disease information+biological information are discussed for a case in which diabetes is concerned. However, in reality, the vectors are set taking into consideration all kinds of diseases covered by the present disclosure. For example, to deal with gout, the dietary restriction condition may include a purine vector. That is, the matrix C may include various component values so as to deal with all kinds of diseases. For example, for a user who is diabetic but does not have gout, the component values of the matrix C related to purine are set to 0 or a value close to 0 via the learning described above. Thus, it is possible to calculate the dietary restriction condition according to equation (1) shown in FIG. 37 for a specific disease of a specific user. Furthermore, according to equation (1) in FIG. 37, the matrix including the disease information+biological information indicates the current state of the user, and thus an appropriate dietary restriction condition can be determined according to the progress of the disease of the user. In the above-described example, the matrix C is set by way of example in consideration of all kinds of diseases treated by the present embodiment. However, the matrix C may be calculated for each disease such as diabetes, gout, etc. In this case, the first server 200 and the information terminal 100 may calculate the matrix C by properly using meal history information related to each disease.

In the case where the first server 200 calculates the matrix C, when an acquisition request for the disease information and/or biological information is received from the information terminal 100 in step S506 in FIG. 30, the first server 200 may transmit the disease information and/or biological information together with the latest matrix C related to the user to the information terminal 100. Thus, the information terminal 100 can calculate the dietary restriction condition using the latest matrix C.

The matrix C may be updated sequentially as the disease of the user progresses. For example, when the information indicating the stage of the disease (for example, the disease type or category) is updated in the information 2600 including the diagnosis contents of the medical certificate, the matrix C may be updated. The information indicating the degree of dietary restriction may be updated when the matrix C is updated. In this case, the information terminal 100 may generate a personalized menu using the information indicating the latest degree of dietary restriction obtained as a result of the updating.

The matrix C may be updated at least one of the following timings: when a result (a medical record) of a periodic medical examination is acquired; or when biological information of the user is acquired by a biosensor 600 of the user. For example, the matrix C may be calculated at predetermined intervals of, for example, 1 day, 2 days, 1 week, 1 month, or the matrix C may be calculated when a personalized menu is generated. The information indicating the degree of dietary restriction may be updated when the matrix C is updated. In this case, the information terminal 100 may generate a personalized menu using the information indicating the latest degree of dietary restriction obtained as a result of the updating.

FIG. 38 is a diagram showing equation (2) for calculating the priority of dish included in menu information. In this equation (2), c1, c2, c3, and c4 are weighting coefficients applied to calories, alcohol, salt, and dietary fiber, respectively, in evaluating dishes.

f1( ), f2( ), f3( ), and f4( ) are functions for determining the degree of satisfying the dietary restriction condition imposed on a user for each dish included in the menu information in terms of calories, alcohol, salt, and dietary fiber, respectively. These functions are explained below by taking the amount of calorie as an example. f1(amount of calorie, amount-of-calorie vector) represents the degree of matching between the amount of calorie included in the ingredient information 2800 of a dish of interest and the amount-of-calorie vector calculated according to equation (1). The degree of matching of the amount of calorie increases as the amount of calorie of the dish of interest approaches the recommended amount of the calorie of the amount-of-calorie vector. This also applies to the amount of salt. The degree of matching of the amount of alcohol increases as the amount of alcohol in the dish of interest becomes closer to the permitted amount or becomes smaller than the prohibited amount. The degree of matching of the amount of dietary fiber increases as the amount of dietary fiber of the dish of interest becomes closer to the recommended amount or increases beyond the required amount. The nutrient vector of a dish of interest includes the amount of calorie, the amount of alcohol, the amount of salt, and the amount of dietary fiber of the dish of interest. These amounts identified from the ingredient information 2800 of the dish of interest.

The information terminal 100 calculates the priority score of each dish included in the menu information by applying the dietary restriction condition calculated according to equation (1) to equation (2), and generates a personalized menu such that the higher the priority score of the dish, the higher the priority of displaying the dish.

When the maximum acceptable intake of calories in the current meal is calculated by the method described above with reference to FIG. 23, the amount-of-calorie vector may be calculated from the calculated maximum acceptable intake of calories in the current meal, and the priority of each dish may be calculated by applying the calculated amount-of-calorie vector to equation (2). When the maximum acceptable intake and the target intake in the current meal are calculated by the method described above with reference to FIG. 23, the information terminal 100 may calculate vectors (for example, the amount-of-alcohol vector, the amount-of-salt vector, the amount-of-dietary-fiber vector) related to a specific ingredient from the calculated maximum acceptable intake and the target intake in the current meal. The information terminal 100 may further calculate the priority of each dish by substituting the calculated vectors related to the specific ingredient into equation (2).

This makes it possible for the information terminal 100 to generate a personalized menu such that dishes including smaller amounts of nutrient whose intake is restricted in relation to a disease of a user are displayed with higher priorities than priorities with which dishes including larger amounts of such nutrient are displayed.

It is also possible for the information terminal 100 to generate a personalized menu such that dishes including larger amounts of nutrient whose intake is recommended for a disease of a user are displayed with higher priorities than priorities with which dishes including smaller amounts of such nutrient are displayed.

The displaying with a lower priority may be performed by at least one of the following manners: an operation on the operation unit 106 by a user to display a dish on the display 105 is more complicated or include a large number of steps for a case where the dish has a lower priority than for a case where the dish has a higher priority; dishes with lower priorities are displayed at positions lower in display order than dishes with higher priorities; a size of an area in which a lower-priority dish is displayed is smaller than a size of an area in which a higher-priority dish is displayed; or a lower-priority dish is displayed in a lower gradation level than a gradation level in which a higher-priority dish is displayed. Examples of implementations of information process performed when dish is ordered via personalized menu

Next, examples are described of implementations of the information process performed in a situation in which a dish is selected from a personalized menu and ordered. If an interface of information communication and a data structure of data handled thereby are unique to a specific restaurant store, this may result in a problem that various kinds of data handled in the information processing system can be used only in the specific store, for example, in the store 40 of the restaurant company A, but cannot be used in other restaurants such as restaurants of the restaurant company B, or a situation may occur in which they cannot be used not only in restaurants of the restaurant company B but even in stores of the restaurant company A other than the store 40. In order to avoid such a situation, a general solution is described below for many users to order dishes using personalized menus in many restaurants.

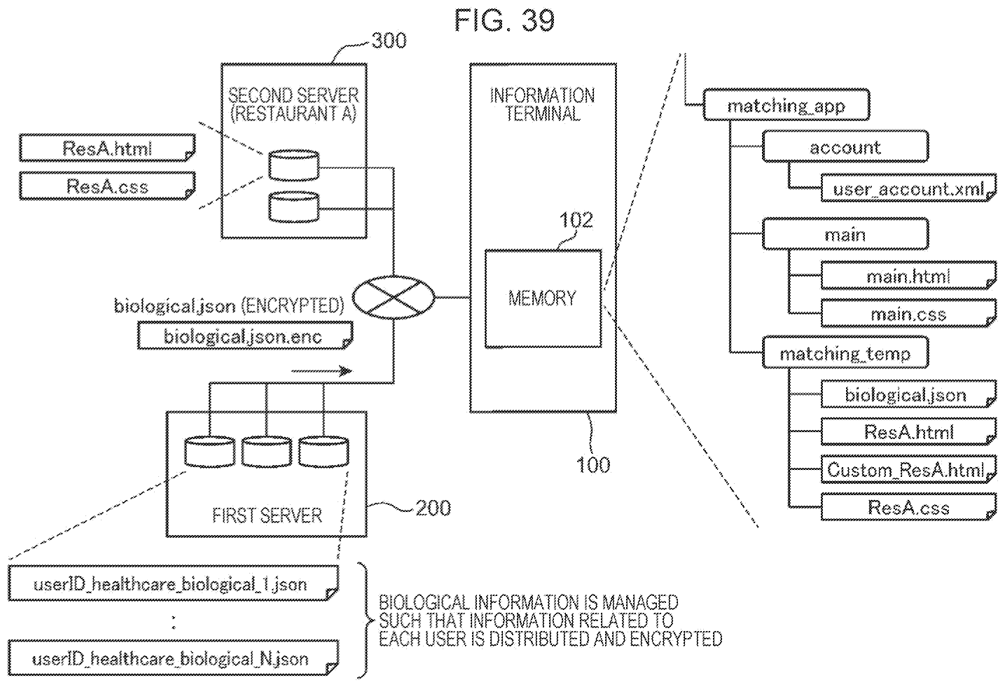

FIG. 39 is a diagram showing an example of a specific implementation of the information processing system according to the present embodiment. The memory 102 of the information terminal 100 has a “matching_app” directory, which is a storage location for files required in executing the matching application. Under the “matching_app” directory, there are “account”, “main”, and “matching_temp” directories. The “account” directory provides a storage location for storing a user's account and/or information required in user authentication are stored. The “main” directory provides a storage location for storing information necessary for the matching application to realize basic functions such as drawing the home screen, scanning QR codes, and the like. The “matching_temp” directory provides a storage location for temporarily storing information required in matching.

The “account” directory provides a storage location for storing a “user_account.xml” file in which account information and/or information required in user authentication are described. More specifically, in the “user_account.xml” file, information for identifying a user, such as a unique account name (for example, a user ID specified by the user) and authentication information related to the account name (for example, a password, a fingerprint feature value, and/or a face feature value), is stored in an encrypted form.

The account name is not limited to the user ID specified by the user, and other kinds of information may be used if they are capable of individually identifying users who use the matching application. For example, a unique serial code for each individual matching application may be used, wherein the unique serial code may be embedded in the matching application program or provided along with the matching application. The serial code unique to each individual matching application is a serial code uniquely assigned to each information terminal 100 on which the matching application is installed. Alternatively, as the account name, a unique account name may be generated based on a random number by the matching application when the matching application is started or registered for the first time, and this generated unique account name may be used. In this case, the matching application may automatically generate the account name after confirming with the first server 200 that the account name is not duplicated with an already registered account name.

By setting, as the account name, character string information that is meaningless when seen by a person such as that described above, it is possible to transmit personal information with higher confidentiality. Note that the disease information and/or biological information included in the personal information is managed such that it is fragmented into a plurality of files as described later. The above-described account name may be used in a user ID part of a file name in each fragmented file. Alternatively, another piece of information may be paired one-to-one with each account name described above and may be used as part of a file name (for example, a user ID part) of each fragmented file.

The “main” directory provides a storage location for storing a “main.html” file in which content information necessary in realizing basic functions of the matching application is described, and a “main.css” file in which a style of a screen display (for example, a UI design) is described.

In the second server 300 of the restaurant company A, a group of files are stored in advance for use in returning a response when a URL (for example, http://restaurantA.com/QRorder-18) represented by a character string read by an QR code reader is accessed. This file group includes a “ResA.html” file in which content information to be returned is described, and a “ResA.css” file in which a style of a screen display (for example, a UI design) of the content information is described. For example, the ingredient information 2800 such as that shown in FIGS. 28A to 28C or FIGS. 29A to 29C may be included in the “ResA.html” file or it may be stored in an external file referred to by the “ResA.html” file.

In the first server 200, a wide variety of personal information of the user, having a huge data size, are accumulated in a distributed and encrypted manner. For example, the biological information related to the user used in the present disclosure is fragmented into a total of N files in the JSON format including a “userID_healthcare_biological_tjson” file, a “userID_healthcare_biological_2.json” file, . . . , a “userID_healthcare_biological_N.json” files, and they are stored in physically different storage apparatuses in the first server 200. In each of the N files, “userID” at the beginning of a file name is identification information for identifying a user of interest, the following “health care” is identification information for identifying that it has an attribute related to health, the following “biological” is identification information for identifying that it is biological information, and a number at the end of the file name is an identification number of a fragmented file. The disease information is also distributed and encrypted in the same manner as the above-described biological information. In this case, in the file name, “sick” is used instead of “biological” to indicate that the file is of disease information.

When the first server 200 receives a request for biological information related to a user together with an appropriate permission (for example, permission information), the first server 200 can correctly restore the data from these N files and convert it into a predetermined description format (.json) thereby obtaining a “biological.json” file, and further encrypt this “biological.json” file to a “biological.json.enc” file and return the resultant file to the matching application. This also applies to the disease information.

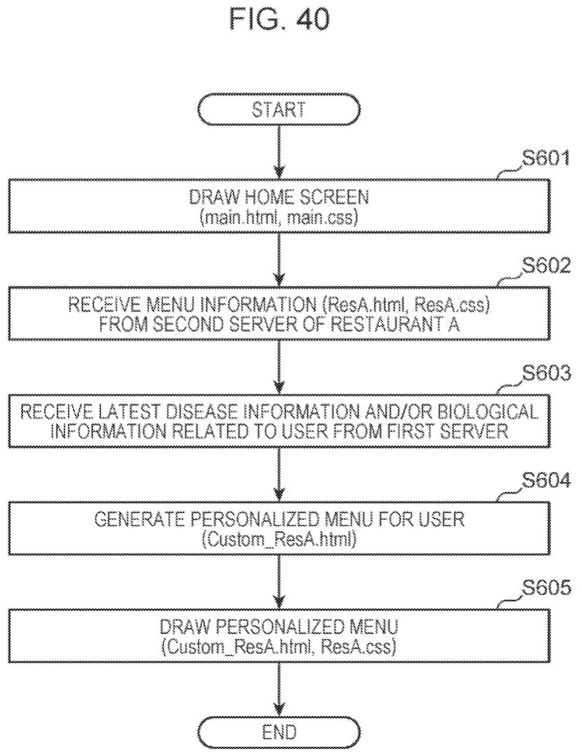

Referring to a flowchart shown in FIG. 40, a description is given below as to handling of files in a situation in which the matching application controls the screen using HTML. FIG. 40 is a flowchart showing an example of a process performed on files by the matching application from the start of the matching application to displaying of a personalized menu.

In step S601, the matching application starts and draws the home screen. More specifically, immediately after the start, the matching application draws the home screen using the “main.html” file and the “main.css” file stored in the “main” directory. As a result, the home screen G103 shown in FIG. 14 is drawn.

In step S602, the matching application receives menu information from the second server 300 of the restaurant company A. The received menu information is stored as a “ResA.html” file and a “ResA.css” file in the “matching_temp” directory.

In step S603, the matching application receives encrypted disease information and/or biological information related to a user from the first server 200. The received disease information and/or biological information is decrypted by the matching application and stored as a “biological.json” file and/or a “sick.json” file in the “matching_temp” directory.

In step S604, the matching application generates a personalized menu for the user so as to meet the information indicating the degree of dietary restriction calculated from the disease information and/or biological information of this user by editing the ResA.html file. The generated personalized menu is newly stored as a “Custom_ResA.html” file in the “matching_temp” directory. Thus, as shown in FIG. 39, the “biological.json” file, the “ResA.html” file, the “Custom_ResA.html” file, and the “ResA.css” file are stored in the “matching_temp” directory. Although not shown in FIG. 39, a “sick.json” file is stored in the “matching_temp” directory.

In step S605, the matching application draws the generated personalized menu in the style specified by the restaurant company A using the “Custom_ResA.html” file and the “ResA.css” file.

Various screens are drawn using HTML/CSS files in the manner described above. Therefore, when a single matching application presents a product or service that matches the vast and diverse personal information of the user among products or services provided by an unspecified number of companies, it is possible to display information expected by a particular company in a style (for example, a UI design) expected by the company.

When ordering of a dish from a personalized menu by a user is completed and the display screen is returned to the home screen of the matching application, or when a predetermined time has passed since the order for the dish from the personalized menu is completed, all files temporarily stored in the “matching_temp” directory may be deleted for safety.

### Variations of Personalized Menu

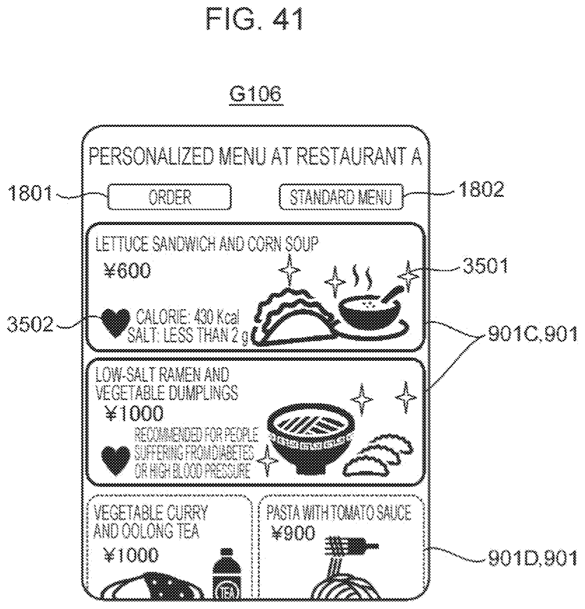

The personalized menu may be generated in various manners variations instead of the manner shown in FIG. 18. Some examples of variations of the manner are described below. FIG. 41 is a diagram showing an example of an operation screen G106 including a first variation of a personalized menu. In this personalized menu, various settings are made on each of tile objects 901 of various dishes in terms of a display order, a size, a thickness of a frame, a decoration of a frame, a size of an image representing a dish, a mark 3501 for deforming the dish around the image representing the dish, a size of a character string of a dish name, deforming of the character string of the dish name, and a heart mark 3502 indicating a favorite dish.

The settings of this personalized menu are made according to the priority described later. In the example shown in FIG. 41, a dish of lettuce sandwich and corn soup has the highest priority, followed by low-salt ramen and vegetable dumplings. Therefore, on the initial display screen of the personalized menu, a tile object 901C of lettuce sandwich and the corn soup is placed at the top, and a tile object 901C of low-salt ramen and vegetable dumplings is displayed at a second position below the top. Vegetable curry and oolong tea has a third priority, and pasta with tomato sauce has a fourth priority, and thus these tile objects 901D are displayed below the tile objects 901C. Tile objects 901 of dishes having a priority equal to or lower than a fifth priority are displayed on the display 105 when the display screen is scrolled.

The two tile objects 901C of dishes with the top priority are set such that the size is set to be larger (for example, twice greater) than the size of default tile objects 901D, decorated to thicken the frame, and decorated to thicken the character string indicating the dish name. Furthermore, deforming marks 3501 are displayed, the size of an image representing the dish is set to be larger (for example, twice larger) than the sizes of tile objects 901D, and a heart mark 3502 is displayed. In addition, the tile object 901C in the first row includes a text message “CALORIE: 430 Kcal; SALT: LESS THAN 2 g” to notify the user that the dish is relatively low in calories and low in salt content. Thus, a user having a dietary restriction can order this dish without having an anxiety about this dish. The tile object 901C in the second row includes a text message “RECOMMENDED FOR PEOPLE SUFFERING FROM DIABETES OR HIGH BLOOD PRESSURE”. This message informs the user that this dish is suitable for people with diabetes or hypertension. A message may be determined in advance for each dish.

In the example descried above, dishes with a priority up to top two priorities are displayed in the form of the tile object 901C, but use of the form of the tile object 901C is not limited to highest two priorities. That is, the form of the tile object 901C may be used for up to three or four highest priorities.

In the example shown in FIG. 41, the width of the tile object 901C is set to be substantially the same as the width of the operation screen G106. On the other hand, the width of the tile object 901D is set to be about half the width of the tile object 901C. As a result, the tile objects 901D with the 3rd and 4th priorities are displayed in the same row. As with the tile objects with the 3rd and 4th priorities, the tile objects with the 5th or lower priorities are displayed in the order of priority such that two tile objects 901D are displayed in the same row. For each two tile objects displayed in the same row, for example, a tile object with a higher priority is displayed on the left side while a tile object with a lower priority is displayed on the right side Vertical lengths of tile objects are set to be equal for the tile object 901C and the tile object 901D.

In order to achieve the displaying of the personalized menu according to the priority, the computational processing unit 104 calculates the priority score by applying equation (2) to each of dishes included in the menu information. The computational processing unit 104 then generates image data of the operation screen G106 including the personalized menu such that the tile objects 901 of the dishes are arranged in descending order of the priority score according to predetermined layout information related to the personalized menu. The layout information may include information specifying, for example, the arrangement position of the tile object 901 according to the priority score and the decoration content according to the priority score for each dish.

Of the generated image data of the personalized menu, image data related to objects inside a display area of the display 105 is displayed on the display 105 as an initial screen image. When an operation of scrolling the display is performed, the computational processing unit 104 may scroll the personalized menu by downwardly sliding the display area of the personalized menu.

In the example shown in FIG. 41, the setting of the tile objects 901C is made in terms of the displaying order, the size, the frame thickness, the frame decoration, the size of the image representing the dish, the mark 3501 that deforms the dish around the image representing the dish, the size of text string representing the dish name, the deformation of the text string representing the dish name, and the heart mark 3502 indicating a favorite dish. However, the setting may be made for at least one of these.

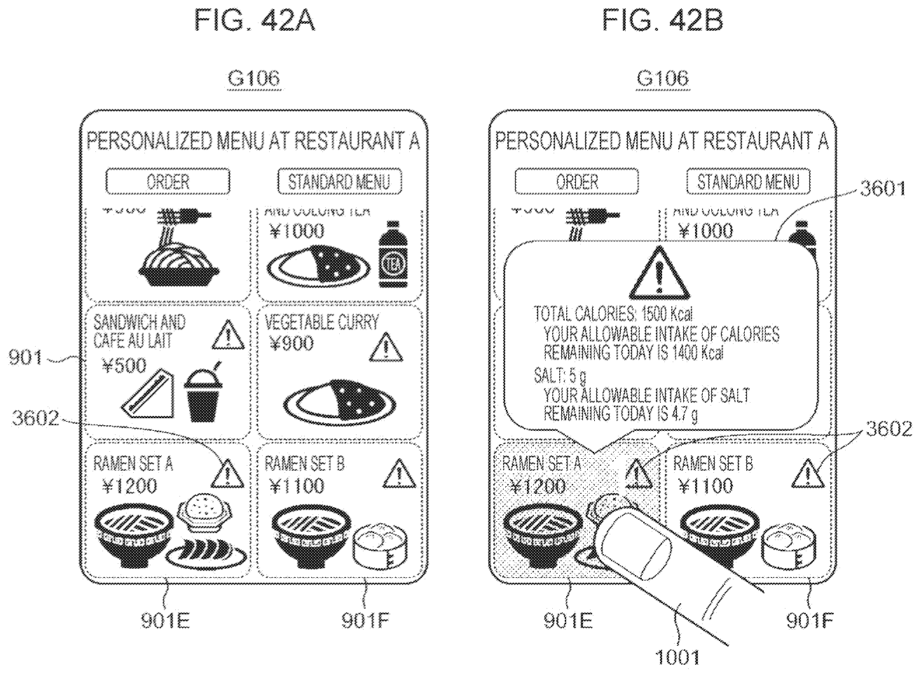

FIGS. 42A and 42B are diagrams showing an example of an operation screen G106 including a second variation of a personalized menu. In this example, a manner of displaying dishes with low priority scores is shown. FIG. 42A shows the operation screen G106 in a state in which a tile object 901 is not yet selected by a user, while FIG. 42B shows the operation screen G106 in a state in which the tile object 901 is selected by the user. On this personalized menu, for dishes that are likely to exacerbate the disease of the user, a caution mark 3602 for calling attention is displayed in each tile objects 901 of such dishes. When a tile object 901E including the caution mark 3602 is touched with a pointing element 1001, a balloon frame 3601 including a caution message is displayed in association with the touched tile object 901E. In this specific example, the tile object 901E of ramen set A is selected. However, this dish contains a large amount of calories and a large amount of salt. Therefore, in the balloon frame 3601, a text message is displayed to indicate the total calories of the dish (1500 Kcal) and the maximum acceptable intake of calories remaining today before eating this dish (1400 Kcal). In addition, the balloon frame 3601 also include a message indicating the salt content of this dish (5 g) and the maximum acceptable intake of salt remaining today before eating this dish (4.7 g). Thus, based on the information presented here, the user can determine whether or not to order this dish. This makes it possible for the user to smoothly order a dish.

In order to realize the above, the computational processing unit 104 performs control such that a caution mark 3602 is displayed on a tile object 901 of each of dishes whose priority score calculated according to equation (2) is equal to or lower than a predetermined first threshold value. When this tile object 901 is touched, the computational processing unit 104 extracts parameters whose matching degree shown by f1( ) to f4( ) of equation (2) is equal to or lower than a predetermined criterion matching degree from the ingredient information 2800 of the corresponding dish, and the computational processing unit displays messages according to the extracted parameters in the balloon frame 3601. Furthermore, the computational processing unit 104 may calculate the maximum acceptable intake as of the current time of the day for the parameters extracted in the manner described above with reference to FIG. 23, and may display the calculated maximum acceptable intake in the balloon frame 3601.

The computational processing unit 104 may display the caution mark 3602 only on the personalized menu or may display the caution mark 3602 also on the standard menu. In a case where the caution mark 3602 is displayed on the standard menu, the computational processing unit 104 may display the caution mark 3602 in a display mode different from the manner in which the caution mark 3602 is displayed on the personalized menu. For example, the caution mark 3602 displayed on the personalized menu may be larger in size than the caution mark 3602 displayed on the standard menu, or may be displayed in a prominent color, or in a prominent pattern.

When the priority score calculated for a dish according to equation (2) is equal to or smaller than a second threshold value smaller than the first threshold, the computational processing unit 104 may display a prohibition mark 3603 on a tile object 901 of this dish instead of the caution mark 3602. Alternatively, the computational processing unit 104 may hide the tile object 901 of the dish for which the priority score calculated according to equation (2) is equal to or smaller than a second threshold value smaller than the first threshold.

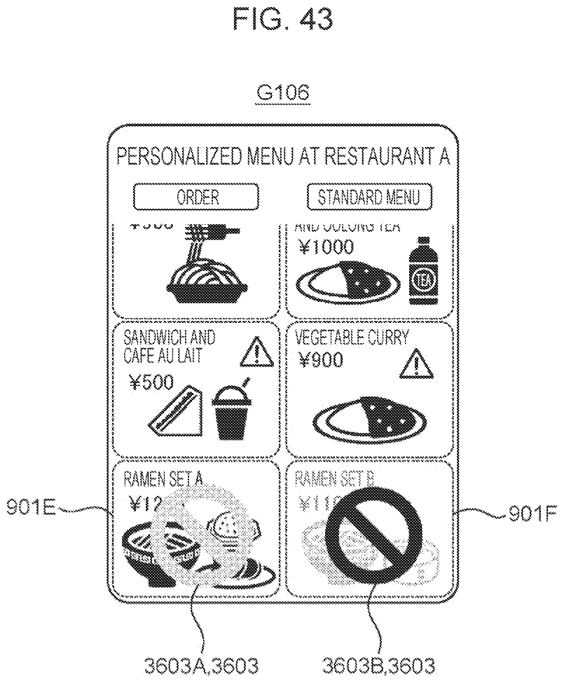

Alternatively, for a dish whose priority score calculated according to equation (2) is equal to or smaller than the second threshold value, the computational processing unit 104 may display a prohibition mark 3603 on the tile object 901 of the dish and disable the selection thereof with the pointing element 1001. In this case, the tile object 901 of this dish may be grayed out. FIG. 43 is a diagram showing an example of an operation screen G106 including a tile object displayed in a grayed out manner. In this example, a prohibition mark 3603A displayed on a tile object 901E is an example of a prohibition mark 3603 displayed on a tile object 901 without graying out the tile object 901, while a prohibition mark 3603B displayed on a tile object 901F is an example of a prohibition mark 3603 displayed on a tile object 901 that is grayed out. The prohibition mark 3603A is displayed in a lighter color than the prohibition mark 3603B. This is to facilitate the distinction between the prohibition mark 3603A and an image representing a dish displayed in the tile object 901E. In the example shown in FIG. 43, both the prohibition mark 3603A and the prohibition mark 3603B are displayed at the same time, but in an alternative mode, either one may be displayed.

The tile object 901F is grayed out by displaying an image representing a dish in a semi-transparent manner. In this example, the character string indicating the dish name is not displayed in the semi-transparent manner, but it may also be displayed in the semi-transparent manner.

When a tile object 901 on which the caution mark 3602 or the prohibition mark 3603 is displayed is touched with the pointing element 1001, the computational processing unit 104 may drive a vibrator provided in the information terminal 100 to vibrate the information terminal 100. This makes it possible to more clearly inform the user that the user has selected a dish containing an ingredient that should be avoided.

FIGS. 44A and 44B are diagrams each showing an example of an operation screen G106 including a third variation of a personalized menu. This personalized menu operates such that when a tile object 901 of a dish is touched by a user with a pointing element 1001, the personalized menu proposes the user to select a dish containing a nutrient that is likely to be deficient for the user. FIG. 44A shows the operation screen G106 in a state in which the object 901 is not yet selected by the user, while FIG. 44B shows the operation screen G106 in a state in which the tile object 901 is selected by the user.

Dietary fiber is an example of a nutrient that tends to deficient. Natto contains a lot of dietary fiber. Thus, in this example, when a tile object 901G of “mushroom pasta” is touched with the pointing element 1001, the computational processing unit 104 displays a balloon frame 3801 in association with the tile object 901G thereby suggesting additionally ordering natto. In the balloon frame 3801, in addition to a message “AMOUNT OF DIETARY FIBER IS NOT ENOUGH!”, messages are displayed to indicate that adding natto provides additional dietary fiber of 3 g and that further intake of 7 g of dietary fiber is required to achieve target intake on this day as of before eating the meal. This suggests that additionally ordering of natto allows the user to efficiently supplement dietary fiber.

The balloon frame 3801 includes a NO button 3804 and a YES button 3805. When the YES button 3805 is touched with pointing element 1001, the computational processing unit 104 determines that natto has been additionally ordered, and changes the order content from the “mushroom pasta” alone to a combination of “mushroom pasta” and “natto”. An information mark 3802 may be displayed on a tile object 901 of a dish for which such a balloon frame 3801 is to be displayed depending on a situation.

In a case where the NO button 3804 is touched with the pointing element 1001, the computational processing unit 104 does not change the order contents.

The computational processing unit 104 may determine, for example, whether or not the amount of nutrient such as dietary fiber that the user tends to lack contained in a dish is equal to or smaller than a third threshold value predetermined according to the disease of the user by referring to the ingredient information 2800 related to the dish, and when it is determined that the amount is equal to or smaller than the third threshold value, the computational processing unit 104 may display the information mark 3802 in association with the dish. In a case where a dish with the information mark 3802 is selected, the personalized menu displays a suggestion for improving a nutrient balance and supporting an improvement of the disease

### Second Embodiment

In the first embodiment described above, the personalized menu is generated by the information terminal 100, but in a second embodiment described below, the personalized menu is generated by the second server 300. The second embodiment in which the personalized menu is generated by the second server 300 is described below in detail.

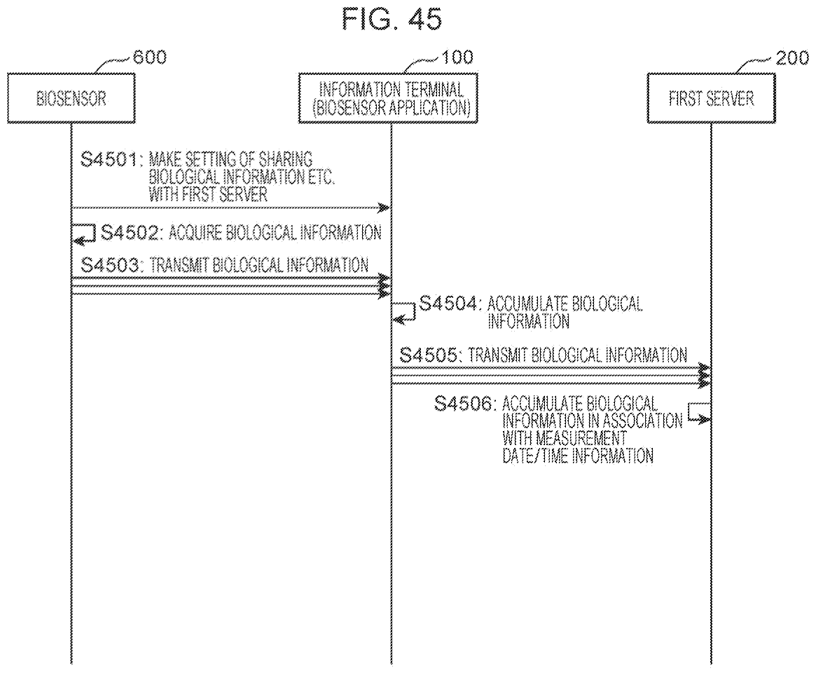

FIG. 45 is a sequence diagram showing an example of a process of uploading biological information to the first server 200 in a mode in which a personalized menu is generated by the second server 300. In step S4501, the biosensor 600 transmits, to the information terminal 100, setting information to allow sharing of biological information with the first server 200. The process of the information terminal 100 described below is executed by a biosensor application installed on the information terminal 100. In step S4502, the biosensor 600 acquires biological information. Note that the biosensor 600 repeatedly acquires biological information. In step S4503, the biosensor 600 sequentially transmits the repeatedly acquired biological information to the information terminal 100.

In step S4504, the information terminal 100 sequentially stores, in the memory 602, the biological information sequentially received from the biosensor 600. In step S4505, the information terminal 100 sequentially transmits the biological information sequentially received from the biosensor 600 to the first server 200. In step S4506, the first server 200 stores the received biological information in the memory 203 in association with the measurement date/time information indicating the date/time of measurement of the biological information. Thus, when the user gives the permission of sharing the biological information, the biological information measured by the biosensor 600 is repeatedly accumulated in the first server 200.

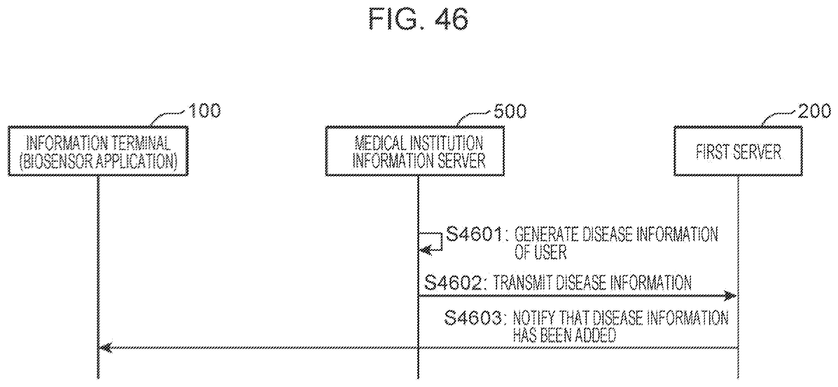

FIG. 46 is a sequence diagram showing an example of a process performed by the first server 200 to acquire disease information in the mode in which a personalized menu is generated by the second server 300.

In step S4601, the medical institution information server 500 generates disease information related to a user based on a result of diagnosis by a doctor or the like. The disease information includes the diagnosis result of the periodic medical examination and/or the diagnosis contents of the disease diagnosis.

In step S4602, the medical institution information server 500 transmits the disease information to the first server 200. In step S4603, in response to receiving the disease information, the first server 200 stores the disease information in the memory 203, and notifies the information terminal 100 that the disease information has been added. Thus, each time the medical institution information server 500 generates disease information, the generated disease information is stored in the first server 200.

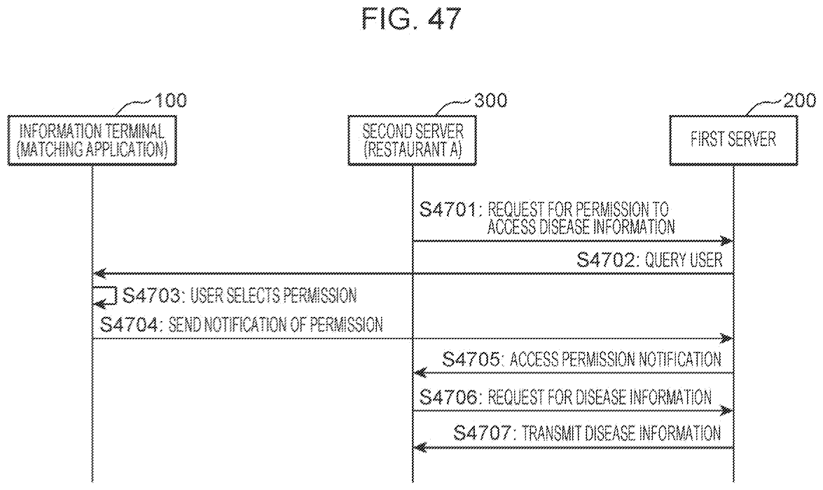

FIG. 47 is a sequence diagram showing an example of a process performed in the information processing system when a user gives the second server 300 a permission to access disease information.

In step S4701, the second server 300 of the restaurant company A issues, to the first server 200, a request for a permission to access disease information. In step S4702, the first server 200 asks the information terminal 100 whether the access is permitted. In step S4703, the information terminal 100 receives a selection instruction from the user to permit access in response to the asking. In step S4704, the information terminal 100 sends an access permission notification to the first server 200 to notify that the access to the disease information is permitted by the user.

In step S4705, the first server 200 sends the access permission notification to the second server 300. In step S4706, the first server 300 requests the first server 200 to provide disease information. In step S4707, the first server 200 sends the disease information to the second server 300.

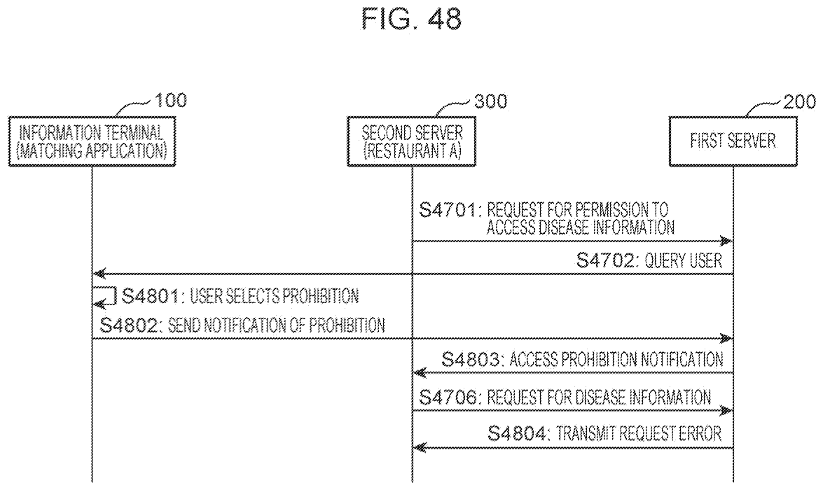

FIG. 48 is a sequence diagram showing an example of a process performed in the information processing system when a user prohibits the second server 300 to access disease information. In FIG. 48, the same processes as those in FIG. 47 are designed by the same process numbers. In step S4801 following step S4702, the information terminal 100 receives, in response to the asking, from the user a selection instruction indicating that the access is to be prohibited. In step S4802, the information terminal 100 sends an access prohibition notification to the first server 200 to notify that the access to the disease information is prohibited by the user.

In step S4803, the first server 200 sends the access prohibition notification to the second server 300. In step S4804 following step S4706, the first server 200 sends a request error to the second server 300 to notify that the disease information is not allowed to be sent to the second server 300.

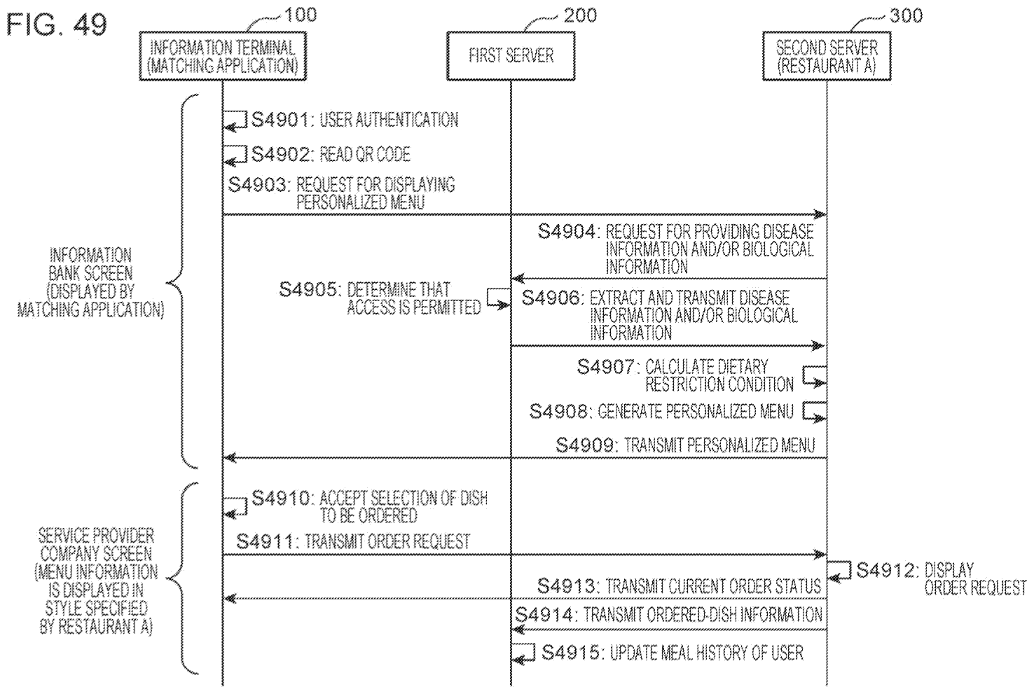

FIG. 49 is a sequence diagram showing an example of a process of generating a personalized menu by an information processing system, in a mode in which personalized menus are generated by a second server 300.

In step S4901, the information terminal 100 performs user authentication. The details of this process are the same as in step S501 shown in FIG. 30. In step S4902, the information terminal 100 performs reads a QR code disposed on a seat. The details of this process are the same as in step S502 shown in FIG. 30. In step S4903, the information terminal 100 requests the second server 300 to display a personalized menu using connection destination information of the second server 300 acquired by analyzing the QR code.

In step S4904, the second server 300 requests the first server 200 to provide disease information and/or biological information. In step S4905, the first server 200 checks whether the second server 300 is permitted to access the disease information. Here, it is assumed that the user has permitted the second server 300 to access the disease information via the process shown in FIG. 47. In step S4906, the first server 200 extracts the disease information and/or biological information of the user of interest from the memory 203 and transmits it to the second server 300.

In step S4907, the second server 300 calculates a dietary restriction condition by substituting the received disease information and/or biological information into equation (1). In step S4908, the second server 300 substitutes the calculated dietary restriction condition into equation (2), calculates the priority score of each dish included in the menu information, and generates a personalized menu in which tile objects 901 of the dishes are arranged according to the calculated priority scores.

In step S4909, the second server 300 transmits the generated personalized menu to the information terminal 100. In step S4910, the information terminal 100 displays a second operation screen including the personalized menu and accepts a selection instruction to select a dish to be ordered.

In step S4911, the information terminal 100 transmits an order request including the ordered-dish information indicating the ordered dish and a seat ID indicating a seat of the user to the second server 300.

In step S4912, the second server 300 displays the order request on a display provided in a store of the restaurant company A. This allows an employee of the store to correctly serve the ordered dish to the seat of the user according to the information indicating the dish and the seat ID displayed on the display.

In step S4913, the second server 300 transmits information indicating the current order status to the information terminal 100. In step S4914, the second server 300 transmits the ordered-dish information included in the received order request to the first server 200. In step S4915, the first server 200 updates the meal history information of the user of interest according to the received ordered dish information.

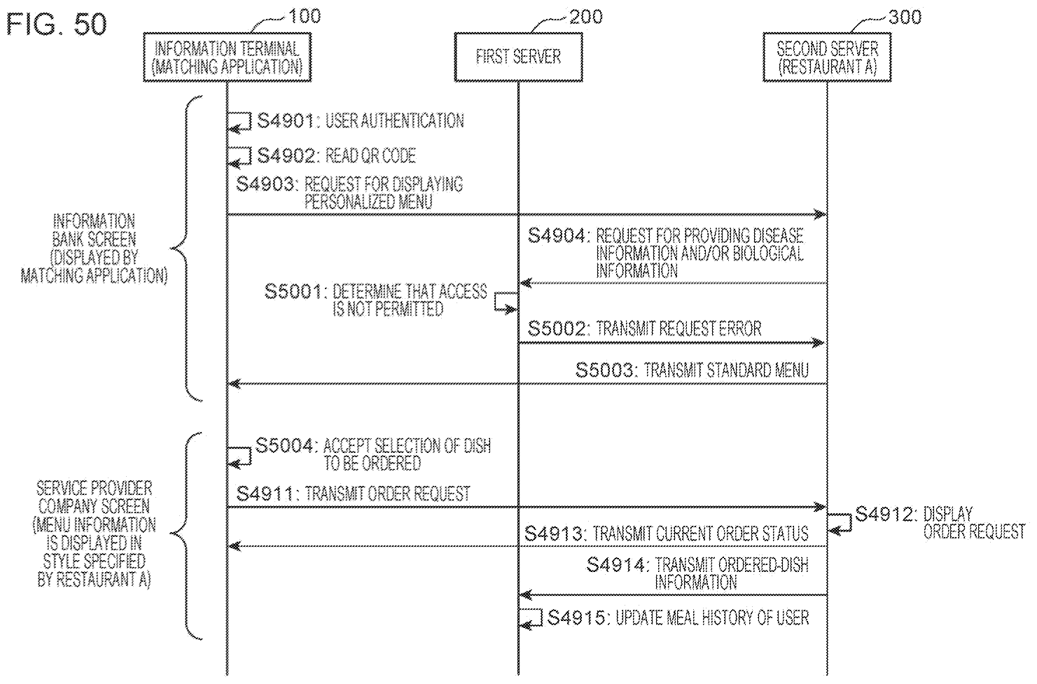

FIG. 50 is a sequence diagram showing an example of a process performed in the information processing system when permission of generating a personalized menu is refused in the mode in which personalized menus are generated by the second server 300. In FIG. 50, the same processes as those in FIG. 49 are designed by the same process numbers, and a further description thereof is omitted.

In step S5001 following step S4904, the first server 200 checks whether the permission to access the disease information has been given to the second server 300. Here, it is assumed that the user has prohibited, via the process shown in FIG. 48, that the second server 300 accesses the disease information. In step S5002, the first server 200 notifies the second server 300 of a request error indicating that the request for the disease information and/or biological information has been rejected. In step S5003, the second server 300 transmits the standard menu to the information terminal 100. In step S5004, the information terminal 100 displays the standard menu and accepts an instruction to select a dish to be ordered. After that, as in FIG. 49, the processes of steps S4911 to S4915 are executed, and the ordered dish is served to the seat of the user.

According to the second embodiment, since the personalized menu is generated by the second server 300, the dish ordered by the user can be surely served to the user's seat while reducing the processing load on the information terminal 100.

### Third Embodiment

In the second embodiment described above, the personalized menu is generated by the second terminal 300, but in a third embodiment described below, the personalized menu is generated by the first server 200. The third embodiment in which the personalized menu is generated by the first server 200 is described in detail below. In the following description of the third embodiment, the same constituent elements as those in the first or second embodiment are designed by the same reference numerals, and a further description thereof is omitted. The first server 200 constitutes a health management system. The health management system constitutes an information processing system.

First, a configuration of the third embodiment is described with reference to FIG. 4. In the third embodiment, personalized menus are generated by the first server 200, and thus the following description focuses on the configuration of the first server 200.

The computational processing unit 202 outputs a request for acquisition of menu information to the second server 300 via the communication unit 201 based on a request for acquisition of a personalized menu for a user transmitted from the information terminal 100.

The computational processing unit 202 acquires a user ID stored in the information terminal 100 from the information terminal 100 via a network and the communication unit 201.

The computational processing unit 202 acquires the menu information from the second server 300 via the communication unit 201.

The computational processing unit 202 reads biological information and/or disease information corresponding to the user ID from the memory 203, and generates information indicating the degree of dietary restriction depending on the progress of the disease of the user based on the read biological information and/or disease information.

The computational processing unit 202 generates a personalized menu for the user based on the menu information and the information indicating the degree of dietary restriction so as to meet the information indicating the degree of dietary restriction. The computational processing unit 202 transmits the personalized menu information indicating the generated personalized menu to the information terminal 100 via the communication unit 201. Thus, the computational processing unit 202 allows the user to select a dish based on the personalized menu on the information terminal 100.

Next, a process performed in the third embodiment is described below. The biological information is uploaded to the first server 200 via a similar process to that shown in FIG. 45. The first server 200 acquires disease information via a similar process to that shown in FIG. 46. In the third embodiment, the second server 300 does not generate personalized menus, and thus the processes shown in FIGS. 47 and 48 are unnecessary.

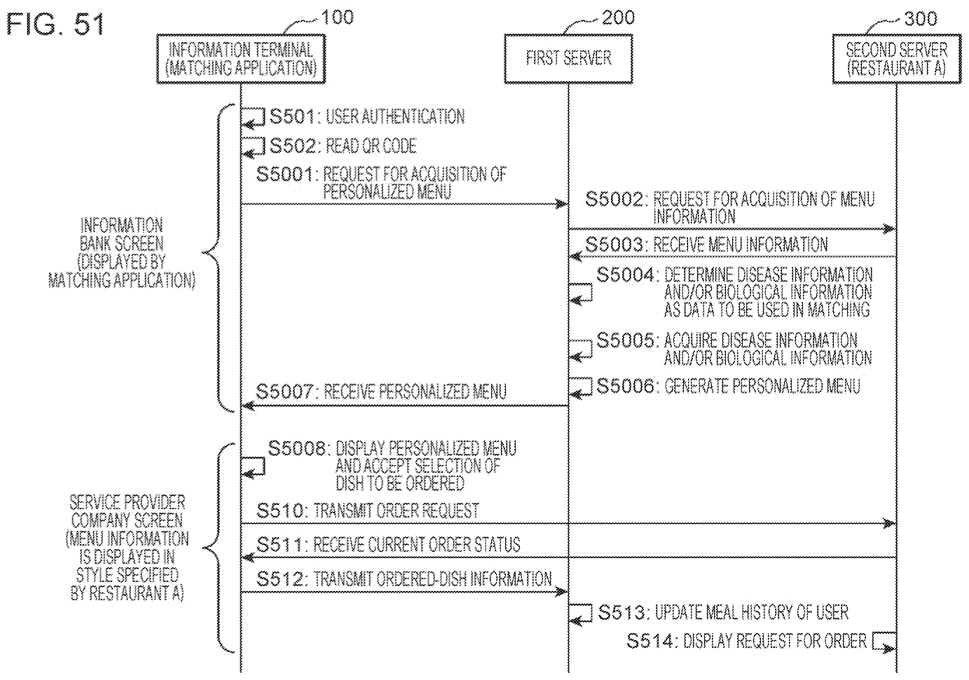

FIG. 51 is a sequence diagram showing an example of a process performed in the information processing system according to the third embodiment. In FIG. 51, similar processes to those in FIG. 30 are designed by the same process numbers.

In step S5001 following step S502, the information terminal 100 (the matching application) transmits a request for acquisition of personalized menu information to the first server 200. The information terminal 100 may transmit a user ID (identification information), a seat ID of a seat of a user, and a store ID of a store of a restaurant company A in which the user is located to the first server 200 together with this acquisition request.

In step S5002, in response to receiving the acquisition request, the first server 200 sends, to the second server 300, a request for acquisition of a menu common to customers (hereinafter, simply referred to as a menu) provided by the store of the restaurant company A or the store indicated by the store ID. In response to receiving this acquisition request, the second server 300 transmits the menu information to the first server 200. Thus, the first server 200 receives the menu information provided by the restaurant company A (step S5003).

When the first server 200 receives the menu information, the first server 200 analyzes the received menu information. In this case, it is detected as a result of the analysis that the data attribute of the received menu information is the food attribute. More specifically, the first server 200 may analyze the contents of the menu information thereby detecting that the menu information has the food attribute. Alternatively, the first server 200 may detect the food attribute from supplementary information transmitted separately from the menu information. When the first server 200 detects that the received menu information has the food attribute as its data attribute, the first server 200 determines that the disease information and/or biological information is to be used as the data for matching (step S5004).

Next, the first server 200 acquires disease information and/or biological information corresponding to the user ID from the memory 203 (step S5005).

The first server 200 then calculates information indicating the degree of dietary restriction based on the acquired disease information and/or biological information, and generates a personalized menu using one of the 17 variations described above based on the calculated information indicating the degree of dietary restriction and the received menu information such that the personalized menu meets the information indicating the degree of dietary restriction of the user (S5006). The first server 200 transmits the personalized menu information indicating the generated personalized menu to the information terminal 100. As a result, the information terminal 100 receives the personalized menu (step S5007).

Various screens displayed in the processes in steps S501, S502, S5001 to S5007 are generated according to the style of the matching application. However, the screen of the personalized menu and other various screens displayed in the dish ordering process may be generated according to a type specified by the restaurant company A.

In response to receiving the personalized menu, the information terminal 100 displays the operation screen G106 including the personalized menu indicated by the personalized menu information in the style specified by the restaurant company A, and accepts an instruction issued by the user to select a dish to be ordered from the personalized menu (step S5008).

After that, the processes in steps S510 to S514 are performed in which a dish is ordered from the personalized menu.

According to the third embodiment, it is possible to reduce the risk that a user with a dietary restriction is erroneously served a dish containing an ingredient that the user should avoid. Furthermore, even for a user for whom it is necessary to make a confirmation of details of ingredients to be avoided, cooking method, etc., the user is allowed to easily order a dish without having a restaurant perform such confirmation, and the restaurant can easily handle the dish order from such a user. Still furthermore, since private information on a user such as disease information and/or biological information is not transmitted to the second server 300, it is possible to reduce the risk that a restaurant staff gets to know the personal information and the risk that the personal information of the user is accumulated in the store terminal.

Note that the embodiments described above are merely examples, and the present disclosure may be applied to various applications.

In the embodiments described above, it is assumed by way of example that the seat of the user is a chair, but the present disclosure is not limited to this. For example, in a stand-up restaurant, one section of a table where the user eats food may be treated as a seat.

In the above-described embodiments, the information indicating the degree of dietary restriction is generated based on the biological information and/or disease information. Note that the information indicating the degree of dietary restriction may be generated only based on the biological information or only based on the disease information.

In each of the above embodiments, each constituent element may be configured by dedicated hardware or may be realized by executing a software program suitable for each constituent element. Each constituent element may be realized by a program execution unit such as a CPU or a processor by reading and executing a software program stored in a storage medium such as a hard disk or a semiconductor memory.

Note that the scope of the present disclosure is not limited by the above-described embodiments. It will be apparent to those skilled in the art that many various modifications may be applicable to the embodiments without departing from the spirit and scope of the present disclosure. Furthermore, constituent elements of different embodiments may be combined. In this case, any resultant combination also falls within the scope of the present disclosure.

According to an example of the control method of the present disclosure, a personalized menu is generated taking into consideration ingredients that a user with dietary restrictions should avoid, which is useful in a dish ordering systems used in restaurants.

What is claimed is:

1. A method of providing information in a health management system that includes a first server which manages biological information of a user in association with identification information identifying the user and that communicates with a second server that stores menu information indicating one or more dishes corresponding to a restaurant, the method comprising:
outputting a request of acquisition of the menu information to the second server based on a request for a personalized menu for the user acquired from an information terminal of the user via a network;
acquiring the identification information stored in the information terminal from the information terminal via a network; acquiring the menu information from the second server;
generating information indicating a degree of dietary restriction depending on a progress of a disease of the user based on the biological information of the user;
generating, based on the menu information and the information indicating the degree of dietary restriction, a personalized menu for the user so as to meet the information indicating the degree of dietary restriction; and
transmitting the personalized menu to the information terminal to allow a dish to be selected via the personalized menu on the information terminal.

2. The method of providing information according to claim 1, wherein
when the request for the personalized menu for the user is acquired, a seat ID indicating a seat of the user is acquired from the information terminal of the user, wherein the seat ID is acquired via an operation screen displayed on a display of the information terminal.

3. The method of providing information according to claim 1, wherein
the personalized menu includes a dish produced by modifying a dish included in the menu information so as to reduce an ingredient specified by the information indicating the degree of dietary restriction as an ingredient to be avoided by the user.

4. The method of providing information according to claim 3, wherein
the reducing of the ingredient includes removing the ingredient such that an amount of the ingredient is zero.

5. The method of providing information according to claim 1, wherein
the personalized menu is generated from the menu information by excluding or graying out a dish including an ingredient specified by the information indicating the degree of dietary restriction as an ingredient to be avoided by the user.

6. The method of providing information according to claim 1, wherein
the personalized menu includes a dish obtained by adding a nutrient, which tends to be deficient depending on the progress of the disease of the user, to a dish included in the one or more dishes.

7. The method of providing information according to claim 1, wherein
the personalized menu includes a description proposing a side dish for supplementing a nutrient tending to be deficient depending on the progress of the disease of the user.

8. The method of providing information according to claim 1, wherein
the personalized menu includes a dish obtained by adding, to a dish included in the one or more dishes, an ingredient including a nutrient lacking in past one or more meals of the user in a particular period.

9. The method of providing information according to claim 1, wherein
the personalized menu includes a description proposing a side dish for supplementing a nutrient lacking in past one or more meals of the user in a particular period.

10. The method of providing information according to claim 1, wherein
the information indicating the degree of dietary restriction includes information indicating a maximum acceptable intake in a current meal as to a specific nutrient related to the disease of the user, and the information indicating the maximum acceptable intake in the current meal is given by a value equal to or smaller than a value obtained by subtracting a cumulative intake taken in past one or more meals in a predetermined period from a maximum acceptable intake in the predetermined period, and
the personalized menu includes a dish obtained such that when a dish included in the one or more dishes contains a greater amount of the specific nutrient than the maximum acceptable intake in the current meal, the amount of the specific nutrient is reduced to be equal to or smaller than the maximum acceptable intake in the current meal.

11. The method of providing information according to claim 1, wherein
the information indicating the degree of dietary restriction includes information indicating a target intake in a current meal as to a specific nutrient related to the disease of the user, and the information indicating the target intake in the current meal is given by a value equal to or greater than a value obtained by subtracting a cumulative intake taken in past one or more meals in a predetermined period from a target intake in the predetermined period, and
the personalized menu includes a dish obtained such that when a dish included in the one or more dishes contains a smaller amount of the specific nutrient than the target intake in the current meal, the amount of the specific nutrient is increased to be equal to or greater than the target intake in the current meal.

12. The method of providing information according to claim 1, wherein
the information indicating the degree of dietary restriction includes information indicating a maximum acceptable intake of calories in a current meal depending on the disease of the user, and the information indicating the maximum acceptable intake of calories in the current meal is given by a value equal to or smaller than a value obtained by subtracting a cumulative intake of calories taken in past one or more meals in a predetermined period from an maximum acceptable intake of calories in the predetermined period, and
the personalized menu includes a dish obtained such that when a dish included in the one or more dishes contains a greater amount of calories than the maximum acceptable intake of calories in the current meal, the amount of calories is reduced to be equal to or smaller than the maximum acceptable intake of calories in the current meal.

13. The method of providing information according to claim 1, wherein
the information indicating the degree of dietary restriction includes information indicating a maximum acceptable intake in a current meal as to a specific nutrient related to the disease of the user, and the information indicating the maximum acceptable intake in the current meal is given by a value equal to or smaller than a value obtained by subtracting a cumulative intake taken in past one or more meals in a predetermined period from a maximum acceptable intake in the predetermined period, and
the personalized menu is generated such that when a dish in the one or more dishes includes a greater amount of the specific nutrient than the maximum acceptable intake in the current meal, the dish is excluded or grayed out.

14. The method of providing information according to claim 1, wherein
the information indicating the degree of dietary restriction includes information indicating a target intake in a current meal as to a specific nutrient related to the disease of the user, and the information indicating the target intake in the current meal is given by a value equal to or greater than a value obtained by subtracting a cumulative intake taken in past one or more meals in a predetermined period from a target intake in the predetermined period, and
the personalized menu is generated such that when a dish included in the or more dishes contains a smaller amount of the specific nutrient than the target intake in the current meal, the dish is excluded or grayed out.

15. The method of providing information according to claim 1, wherein
the information indicating the degree of dietary restriction includes information indicating a maximum acceptable intake of calories in a current meal depending on the disease of the user, and the information indicating the maximum acceptable intake of calories in the current meal is given by a value equal to or smaller than a value obtained by subtracting a cumulative intake of calories taken in past one or more meals in a predetermined period from an maximum acceptable intake of calories in the predetermined period, and
the personalized menu is generated such that when a dish included in the one or more dishes contains a greater amount of calories than the maximum acceptable intake of calories in the current meal, the dish is excluded or grayed out.

16. The method of providing information according to claim 1, wherein
the information indicating the degree of dietary restriction includes information indicating a maximum acceptable intake in a current meal as to a specific nutrient related to the disease of the user, and the information indicating the maximum acceptable intake in the current meal is given by a value equal to or smaller than a value obtained by subtracting a cumulative intake taken in past one or more meals in a predetermined period from a maximum acceptable intake in the predetermined period, and
the personalized menu includes a dish obtained such that when a dish included in the one or more dishes contains a greater amount of the specific nutrient than the maximum acceptable intake in the current meal, the dish is added with an ingredient containing a neutralizing nutrient having an effect that intake of the neutralizing nutrient together with the specific nutrient neutralizes an adverse effect of the greater amount of specific nutrient than the maximum acceptable intake.

17. The method of providing information according to claim 1, wherein
the information indicating the degree of dietary restriction includes information indicating a maximum acceptable intake in a current meal as to a specific nutrient related to the disease of the user, and the information indicating the maximum acceptable intake in the current meal is given by a value equal to or smaller than a value obtained by subtracting a cumulative intake taken in past one or more meals in a predetermined period from a maximum acceptable intake in the predetermined period, and
the personalized menu includes a description of a combination of a dish including a greater amount of the specific nutrient than the maximum acceptable intake in the current meal and a dish including a neutralizing nutrient having an effect that intake of the neutralizing nutrient together with the specific nutrient neutralizes an adverse effect of the greater amount of the specific nutrient than the maximum acceptable intake.

18. The method of providing information according to claim 1, wherein
the information indicating the degree of dietary restriction includes information indicating a maximum acceptable intake in a current meal as to a specific nutrient related to the disease of the user, and the information indicating the maximum acceptable intake in the current meal is given by a value equal to or smaller than a value obtained by subtracting a cumulative intake taken in past one or more meals in a predetermined period from a maximum acceptable intake in the predetermined period, and
the personalized menu includes a description of a combination of dishes that provides a total amount of intake equal to or smaller than the maximum acceptable intake in the current meal.

19. The method of providing information according to claim 1, wherein
the information indicating the degree of dietary restriction includes information indicating a target intake in a current meal as to a specific nutrient related to the disease of the user, and the information indicating the target intake in the current meal is given by a value equal to or greater than a value obtained by subtracting a cumulative intake taken in past one or more meals in a predetermined period from a target intake in the predetermined period, and
the personalized menu includes a description of a combination of dishes that provides intake equal to or greater than the target intake in the current meal.

20. The method of providing information according to claim 1, wherein
the information indicating the degree of dietary restriction includes information indicating a maximum acceptable intake of calories in a current meal depending on the disease of the user, and the information indicating the maximum acceptable intake of calories in the current meal is given by a value equal to or smaller than a value obtained by subtracting a cumulative intake of calories taken in past one or more meals in a predetermined period from an maximum acceptable intake of calories in the predetermined period, and
the personalized menu includes a description of a combination of dishes that provides a total amount of calories equal to or smaller than the maximum acceptable intake of calories in the current meal.

21. The method of providing information according to claim 1, wherein
the personalized menu includes a first dish and a second dish that is displayed at a lower priority than the first dish, wherein the second dish includes a greater amount of nutrient whose intake is restricted due to the disease of the user than the first dish.

22. The method of providing information according to claim 1, wherein
the personalized menu includes a first dish and a second dish that is displayed at a lower priority than the first dish, wherein the second dish includes a smaller amount of nutrient whose intake is recommended for the disease of the user than the first dish.

23. The method of providing information according to claim 21, wherein
displaying of the second dish at the lower priority than the first dish is executed in one of manners including at least a) a manner in which an order of displaying the second dish is lower than an order of displaying the first dish, b) a manner in which a size of an area in which the second dish is displayed is smaller than a size of an area in which the first dish is displayed, or c) a manner in which the second dish is displayed in a lower gradation level than a gradation level in which the first dish is displayed.

24. The method of providing information according to claim 1, wherein
the information indicating the degree of dietary restriction is updated as the disease of the user progresses.

25. The method of providing information according to claim 1, wherein
the information indicating the degree of dietary restriction is updated according to at least one of a) an acquisition of a result of a medical examination, b) an acquisition of medical record information generated when a diagnosis is made, or c) an acquisition of biological information of the user via a biosensor that detects the biological information of the user.

26. The method of providing information according to claim 1, further includes
receiving, from the information terminal of the user, a) information indicating a dish name of a dish selected via the personalized menu, b) information indicating a price of the dish selected in via the personalized menu, c) information indicating a date and time when the dish selected via the personalized menu is ordered, and d) information indicating a nutrient included in the dish selected via the personalized menu.

27. The method of providing information according to claim 1, wherein
the identification information includes a user ID.

28. The method of providing information according to claim 1, wherein
the first server is different from the second server.

29. The method of providing information according to claim 2, wherein
the seat ID is acquired by reading, via the operation screen, an identification code prepared at a corresponding position of a table where the user is seated.

30. The method of providing information according to claim 29, wherein
the identification code includes a QR code.

31. The method of providing information according to claim 29, wherein
the identification code is read using NFC (Near Field Communication).

32. The method of providing information according to claim 1, wherein
the first server manages, in a distributed manner, biological information, preference information of the user including goods purchase history information or dish order history information, and action history information including position information of the user.
